(19) United States
(12) Patent Application Publication
(10) Pub. No.: US 2025/0277256 A1
(43) Pub. Date: Sep. 4, 2025

(54) NUCLEIC ACID DETECTION AND ANALYSIS SYSTEMS

(71) Applicant: The Johns Hopkins University, Baltimore, MD (US)

(72) Inventors: Taekjip Ha, Baltimore, MD (US); Yanbo Wang, Baltimore, MD (US); Wayne T. Cottle, Baltimore, MD (US)

(21) Appl. No.: 18/280,164

(22) PCT Filed: Mar. 1, 2022
(86) PCT No.: PCT/US22/18387

§ 371 Date: Sep. 1, 2023

Related U.S. Application Data
(60) Provisional application No. 63/155,286, filed on Mar. 1, 2021.

(51) Int. Cl. C12Q 1/6827; C12N 15/11; C12Q 1/34; C12Q 1/6806; C12Q 1/6816

(52) U.S. Cl. CPC C12Q 1/6827; C12N 15/11; C12Q 1/34; C12Q 1/6806; C12Q 1/6816; G01N 2333/922

(57) ABSTRACT

In one embodiment, methods for detecting a specific nucleic acid sequence in a genome are provided that may include: a) inducing a nick in genomic nucleic acid sequences by a gene editing complex; b) denaturing the genomic nucleic acid sequences by contacting the genomic nucleic acid sequences with a helicase enzyme at the nicked genomic nucleic acid sequences; c) contacting the denatured genome with a detectably labeled probe, wherein the detectably labeled probe is complementary to the specific nucleic acid sequence of interest; and, d) detecting the specific nucleic acid sequence of interest.

### CROSS-REFERENCE TO RELATED APPLICATIONS

This application is a 35 U.S.C. § 371 national phase entry of International Patent Application No. PCT/US22/18387 filed Mar. 1, 2022, which claims the benefit of priority under 35 U.S.C. 119 from U.S. provisional application No. 63/155,286, filed Mar. 1, 2021 the disclosures of which are incorporated herein by reference in their entireties.

### STATEMENT AS TO RIGHTS TO INVENTIONS MADE UNDER FEDERALLY SPONSORED RESEARCH AND DEVELOPMENT

This invention was made with government support under grant GM122569 awarded by the National Institutes of Health and PHY1430124 awarded by the National Science Foundation. The government has certain rights in the invention.

### INCORPORATION BY REFERENCE OF SEQUENCE LISTING

The contents of the sequence listing text file named “048317-627001WO_SL_123767101_1.txt”, which was created on Mar. 31, 2022 and is 1,912,832 bytes in size, are incorporated herein by reference in its entirety.

### BACKGROUND

New systems for detection and analysis of nucleic acid sequence are sought. Among others, understanding of the relationship between chromosome arrangement and gene expression is limited.

### SUMMARY

In one embodiment, we now provide new methods for detecting a specific nucleic acid sequence in a genome.

Preferred methods may include:
(a) inducing a nick in genomic nucleic acid sequences by a gene editing complex;
(b) denaturing the genomic nucleic acid sequences by contacting the genomic nucleic acid sequences with a helicase enzyme at the nicked genomic nucleic acid sequences;
(c) contacting the denatured genome with a detectably labeled probe, wherein the detectably labeled probe is complementary to the specific nucleic acid sequence of interest; and,
(d) detecting the specific nucleic acid sequence of interest.

In certain aspects, the specific nucleic acid sequence of interest comprises one or more nucleic acid sequences in coding and non-coding nucleic acid sequences of the genome. Suitably, genomic nucleic acid sequences comprise genomic DNA.

In certain aspects, the nicking of genomic DNA sequences by the gene editing complex produces a 3′ single-stranded nucleic acid overhang. In certain aspects, the helicase binds to the genomic DNA at the site of the nick and unwinds downstream double stranded genomic DNA.

In certain aspects, the gene editing complex comprises a Clustered Regularly Interspaced Short Palindromic Repeat (CRISPR)-associated endonuclease and at least one guide nucleic acid sequence.

In certain aspects, the gene editing complex comprises at least two guide nucleic acid sequences.

In certain aspects, the one or more guide nucleic acid sequences are RNA. Suitably, the guide RNA (gRNA) sequences comprise at least about 90% sequence identity to one or more target nucleic acid sequences in coding and non-coding nucleic acid sequences of the genome, or complementary sequences thereof. In certain aspects, the guide RNA (gRNA) sequences are complementary to one or more target nucleic acid sequences in coding and non-coding nucleic acid sequences of the genome, or complementary sequences thereof.

In certain aspects, one or more guide RNAs may be used having one or more nucleotide mismatches compared to the target nucleic acid sequence, or complementary sequences thereof. The one or more single-nucleotide mismatches suitably are in one or more guide RNAs inhibit nicking of target genomic DNA.

In certain aspects, the guide RNA comprises crRNA and tracrRNA.

In certain aspects, the gene-editing complex comprises CRISPR-associated endonuclease is a Type I, Type II, or Type III Cas endonuclease.

In certain aspects, the CRISPR-associated endonuclease is a Cas9 endonuclease, a Cas12 endonuclease, a CasX endonuclease, a CasΦ endonuclease or variants thereof. In certain preferred aspects, the CRISPR-associated endonuclease is a Cas9 nuclease or variants thereof. In certain preferred aspects, the Cas9 nuclease is a Staphylococcus aureus Cas9 nuclease.

In certain aspects, a Cas9 variant comprises a human-optimized Cas9; a nickase mutant Cas9; saCas9; enhanced-fidelity SaCas9 (efSaCas9); SpCas9(K855a); SpCas9(K810A/K1003A/r1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A.

In certain aspects, the helicase is a superhelicase. For example, the superhelicase may comprise: a Super Family 1 (SF 1) helicase, a Super Family 2 (SF2) helicase, a Super Family 3 (SF3) helicase, a Super Family 4 (SF4) helicase, a Super Family 5 (SF5) helicase or a Super Family 6 (SF6) helicase.

In certain aspects, the helicase comprises: a Rep helicase, a UvrD helicase, a Per A helicase or homologs thereof.

In certain aspects, the helicase is a Rep helicase or homologs thereof.

In another embodiment, methods are provided for detecting mutations in a genome of a cell or tissue, comprising:
(a) inducing a nick in genomic DNA by a gene editing complex;
(b) denaturing the genomic DNA by contacting the genome with a helicase enzyme at the nicked genomic DNA;
(c) contacting the denatured genomic DNA with a detectably labeled probe, wherein the detectably labeled probe is complementary to the specific nucleic acid sequence of interest; and,
(d) detecting the mutations in the genome.

In certain aspects, the specific nucleic acid sequence of interest comprises one or more nucleic acid sequences in coding and non-coding nucleic acid sequences of the genome.

In certain aspects of these methods, the cell or tissue is a diagnostic for disease such as cancer.

In certain aspects, the nicking of genomic DNA sequences by the gene editing complex produces a 3′ single-stranded nucleic acid overhang.

In certain aspects, the helicase binds to the genomic DNA at the site of the nick and unwinds downstream double stranded genomic DNA.

In certain aspects, the gene editing complex comprises a Clustered Regularly Interspaced Short Palindromic Repeat (CRISPR)-associated endonuclease and at least one guide nucleic acid sequence.

In certain aspects, the gene editing complex comprises at least two guide nucleic acid sequences.

In certain aspects, the one or more guide nucleic acid sequences are RNA. Suitably, the guide RNA (gRNA) sequences comprise at least about 90% sequence identity to one or more target nucleic acid sequences in coding and non-coding nucleic acid sequences of the genome, or complementary sequences thereof.

In certain aspects, the guide RNA (gRNA) sequences are complementary to one or more target nucleic acid sequences in coding and non-coding nucleic acid sequences of the genome, or complementary sequences thereof.

In certain aspects, one or more guide RNAs are used that have one or more nucleotide mismatches compared to the target nucleic acid sequence, or complementary sequences thereof. Suitably, one or more single-nucleotide mismatches in one or more guide RNAs inhibit nicking of target genomic DNA.

In certain aspects, the guide RNA comprises crRNA and tracrRNA.

In certain aspects, the gene-editing complex comprises CRISPR-associated endonuclease is a Type I, Type II, or Type III Cas endonuclease.

In certain aspects, the CRISPR-associated endonuclease is a Cas9 endonuclease, a Cas12 endonuclease, a CasX endonuclease, a CasΦ endonuclease or variants thereof.

In certain aspects, the CRISPR-associated endonuclease is a Cas9 nuclease or variants thereof.

In certain aspects, the Cas9 nuclease is a Staphylococcus aureus Cas9 nuclease.

In certain aspects, a Cas9 variant comprises a human-optimized Cas9; a nickase mutant Cas9; saCas9; enhanced-fidelity SaCas9 (efSaCas9); SpCas9(K855a); SpCas9(K810A/K1003A/r1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A.

In certain aspects, the helicase is a superhelicase comprising: a Super Family 1 (SF 1) helicase, a Super Family 2 (SF2) helicase, a Super Family 3 (SF3) helicase, a Super Family 4 (SF4) helicase, a Super Family 5 (SF5) helicase or a Super Family 6 (SF6) helicase.

In certain aspects, the helicase comprises: a Rep helicase, a UvrD helicase, a Per A helicase or homologs thereof.

In certain aspects, the helicase is a Rep helicase or homologs thereof.

In the present methods, a labeled probe suitably may comprise any xeno nucleic acid or other modified nucleic acid, including but not limited to: 1,5-anhydrohexitol nucleic acid (HNA), cyclohexene nucleic acids (CeNA), Threose nucleic acids (TNA), glycol nucleic acids (GNA), locked nucleic acids (LNA), peptide nucleic acid (PNA), bridged nucleic acids (BNA), Fluoro Arabino nucleic acids (FANA), or chimeric DNA/RNA.

In the present methods, the labeled probe suitably may be modified with functional groups including but not limited to: polyethylene glycol, cholesterol, fatty acid chains, glycosylation, fluorescent labeling, N6-methyladenosine (m⁶A), N⁶,2′-O-dimethladenosime (m⁶Am), N⁴-acetylcytidine (ac⁴C), 2′-O-methylation, NAD+ cap, inverted dT cap, 2-O′-methy, 2′-deoxy, 2′-hydroxyl, 2′-fluoro, 2′-O-alkyl, 2′-O-alyl, 2′-O-phenyl, 2′-O-sulphur, 2′-carbon linked substitutions, 2′-carbamate linkages, other 2′ sugar substitutions, 5 or 6 pyrimidine substitution, other pyrimidine substitutions, cyclic sugar analogs, and non-phosphorous backbones.

In certain aspects, a method of detecting single nucleotide variation (SNV) mutations in a genome of a cell or tissue, comprises inducing a nick in genomic DNA by a gene editing complex; denaturing the genomic DNA by contacting the genome with a helicase enzyme at the nicked genomic DNA; contacting the denatured genomic DNA with a detectably labeled probe, wherein the detectably labeled probe is complementary to the specific nucleic acid sequence of interest; and, detecting the SNV mutations in the genome. In certain embodiments, the gene editing complex comprises a Clustered Regularly Interspaced Short Palindromic Repeat (CRISPR)-associated endonuclease and one guide nucleic acid sequence. In certain embodiments, the guide RNAis extended by addition of one or more nucleobases at the 5′ or 3′ end. In certain embodiments, the guide RNA is extended at the 5′. In certain embodiments, the specific nucleic acid sequence of interest comprises one or more nucleic acid sequences in coding and non-coding nucleic acid sequences of the genome. In certain embodiments, the guide RNA (gRNA) sequences comprise at least about 90% sequence identity to one or more target nucleic acid sequences in coding and non-coding nucleic acid sequences of the genome, or complementary sequences thereof. In certain embodiments, the guide RNA comprises one or more nucleotide mismatches compared to the target nucleic acid sequence, or complementary sequences thereof. In certain embodiments, the guide RNA comprises crRNA and tracrRNA. In certain embodiments, the gene-editing complex comprises CRISPR-associated endonuclease is a Type I, Type II, or Type III Cas endonuclease. In certain embodiments, the CRISPR-associated endonuclease is a Cas9 endonuclease, a Cas12 endonuclease, a CasX endonuclease, a CasΦ endonuclease or variants thereof. In certain embodiments, the CRISPR-associated endonuclease is a Cas9 nuclease or variants thereof. In certain embodiments, the Cas9 nuclease comprises a Streptococcus pyogenes Cas9 nuclease or a Staphylococcus aureus Cas9 nuclease. In certain embodiments, a Cas9 variant comprises a single nucleotide variation (SNV) optimized Cas9; a human-optimized Cas9; a nickase mutant Cas9, eCas9 H840A; saCas9; enhanced-fidelity SaCas9 (efSaCas9); SpCas9(K855a); SpCas9(K810A/K1003A/r1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A. In certain embodiments, the single nucleotide variation (SNV) optimized Cas9 is eSpCas9(1.1). In certain embodiments, the helicase is a superhelicase comprising: a Super Family 1 (SF 1) helicase, a Super Family 2 (SF2) helicase, a Super Family 3 (SF3) helicase, a Super Family 4 (SF4) helicase, a Super Family 5 (SF5) helicase or a Super Family 6 (SF6) helicase. In certain embodiments, the helicase comprises: a Rep helicase, a UvrD helicase, a Per A helicase or homologs thereof.

In certain embodiments, the CRISPR-associated endonuclease is optimized for expression in a human cell.

In certain embodiments, the isolated nucleic acid sequences are included in at least one expression vector selected from the group consisting of: a lentiviral vector, an adenovirus vector, an adeno-associated virus vector, a vesicular stomatitis virus (VSV) vector, a pox virus vector, and a retroviral vector. In certain embodiments, the expression vector comprises: a lentiviral vector, an adenoviral vector, or an adeno-associated virus vector. In certain embodiments, the adeno-associated virus (AAV) vector is AV1, AAV2, AAV3, AAV4, AAV5, AAV6, AAV7, AAV8, AAV9, AAV10, AAV11, AAVDJ, or AAVDJ/8. In certain embodiments, the vector comprising the nucleic acid further comprises a promoter. In certain embodiments, the promoter comprises a ubiquitous promoter, a tissue-specific promoter, an inducible promoter or a constitutive promoter.

Any compositions or methods provided herein can be combined with one or more of any of the other compositions and methods provided herein.

Other aspects of the invention are disclosed infra.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

As used herein, the term “crRNA” or “guide RNA” or “single guide RNA” or “sgRNA” or “one or more nucleic acid components” of a CRISPR-Cas comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide RNA (gRNA) is a chimeric molecule that consists of tracrRNA and crRNA, anteceded by an 18-20-nt spacer sequence complementary to target DNA before a protospacer adjacent motif (PAM). In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide RNA may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmic RNA (scRNA). In some embodiments, the target sequence may be a sequence within a RNA molecule selected from the group consisting of mRNA, pre-mRNA, and rRNA. In some embodiments, the target sequence may be a sequence within a RNA molecule selected from the group consisting of ncRNA, and lncRNA. In some embodiments, the target sequence may be a sequence within an mRNA molecule or a pre-mRNA molecule.

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residue in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “hybridization” refers to a reaction in which one or more polynucleotides react to form a complex that is stabilized via hydrogen bonding between the bases of the nucleotide residues. The hydrogen bonding may occur by Watson Crick base pairing, Hoogsteen binding, or in any other sequence specific manner. The complex may comprise two strands forming a duplex structure, three or more strands forming a multi stranded complex, a single self-hybridizing strand, or any combination of these.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

A nicking enzyme (or nicking endonuclease) is an enzyme that cuts one strand of a double-stranded DNA at a specific recognition nucleotide sequences known as a restriction site. Such enzymes may hydrolyse (cut) only one strand of the DNA duplex, to produce DNA molecules that are “nicked”, rather than cleaved.

“Protospacer adjacent motif” (PAM) is a 3-nt sequence located immediately downstream of the single guide RNA (sgRNA) target site, which plays an essential role in binding and for Cas-mediated DNA cleavage. The PAMs are the various extended conserved bases at the 5′ or 3′ end of the protospacer.

The term “stringent conditions” for hybridization refers to conditions under which a nucleic acid having complementarily to a target sequence predominantly hybridizes with the target sequence, and substantially does not hybridize to non-target sequences. Stringent conditions are generally sequence-dependent and vary depending on a number of factors. in general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology—Hybridization With Nucleic Acid Probes Part 1, Second Chapter “Overview of principles of hybridization and the strategy of nucleic acid probe assay”, Elsevier, N.Y.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “percent sequence identity” or having “a sequence identity” refers to the degree of identity between any given query sequence and a subject sequence. The term percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more over a region that is 100 to 500, 100 to 200, 150 to 200, 175 to 200, 175 to 225, 175 to 250, 200 to 225, 200 to 250 or more amino acids or nucleotides in length.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 (includes FIGS. 1A-1F) shows Cas9 exposes the NTS 3′ flap for Rep-X loading and unwinding the downstream dsDNA. FIG. 1A: Schematic of Rep-X loaded on the NTS 3′ flap and translocating along the DNA strand. The NTS 3′ flap is outlined in a dash box. FIG. 1B: Schematic of GOLD FISH. The unwound DNA may not rezip behind the translocating helicase for three possible reasons indicated. FIG. 1C: Schematic for the DNA helicase invasion assay. A nick is indicated in the figure at 20 bp downstream of the protospacer. FIG. 1D: Representative images at different time points during the DNA helicase invasion assay. Note that the images were taken at different locations on the slide surface. Scale bar, 5 μm. FIG. 1E: Spot number per imaging view decreased with time in the DNA helicase invasion assay when using Cas9_{dHNH}. FIG. 1F: Spot number per imaging view did not change with time with dCas9 or in the absence of ATP. Error bar represent mean±SD (n=20). n represents number of different imaging views. The experiments were repeated twice independently with similar results.

FIG. 2 (includes FIGS. 2A-2F) shows GOLD FISH targeting a repetitive region within the MUC4 gene (MUC4-R). FIG. 2A: A representative image of GOLD FISH against MUC4-R in IMR-90 cells. A single cell outlined in green is magnified on the upper-right corner. Probes were Cy5-labeled, and nuclei were stained using Hoechst 33342. Scale bar, 10 μm. FIG. 2B: (Left panel) Histogram of number of MUC4 foci detected in each cell (n=78). Percentages of total cells are indicated. (Right panel) Box plot of signal-to-background ratio of detected FISH spots. Mean±SD are represented using a black line and box. Each dot represents one FISH spot (n=163). FIG. 2C: Schematic for GOLD FISH using ATTO550-labeled guide RNA. ATTO550 was conjugated at the 5′ end of tracrRNA. FIG. 2D: Quantification of co-localized loci from ATTO550-guide RNA and Cy5-GOLD FISH probe. The black numbers indicate spot number examined in each channel. FIG. 2E: A representative image of MUC4 fluorescent signals from ATTO550-guide RNA (green) and Cy5-GOLD FISH probes (magenta) in HEK293ft cells. Scale bar, 5 μm. FIG. 2F: Comparison of the signal-to-background ratio of ATTO550 foci and Cy5 foci from the co-localization assay, and CASFISH foci using Cas9_{dHNH} and dCas9. Mean SD are represented using a line and box in the box plot. Each dot represents one FISH spot (n=135, 146, 197 and 154 for ‘ATTO550 foci’, ‘Cy5 foci’, ‘CASFISH foci Cas9_{dHNH}’ and ‘CASFISH foci dCas9’ respectively). ***P<0.001 (Student's t-test).

FIG. 3 (includes FIGS. 3A-3G) shows GOLD FISH targeting a non-repetitive region within the MUC4 gene. FIG. 3A: (Top) A schematic showing Cas9 binding sites and probe targeting region for GOLD FISH against MUC4 non-repetitive region (MUC4-NR) using MUC4-NR guide-RNA set 1. (Bottom) A representative image of GOLD FISH against MUC4-NR and MUC4 repetitive region (MUC4-R) in an IMR-90 cell. Scale bar, 5 μm. FIG. 3B: (Left panel) Histogram of number of MUC4-NR foci detected in each cell (n=78). Percentages of total cells are indicated. (Right panel) Box plot of signal-to-background ratio of detected MUC4-NR FISH spots, mean±SD are represented using a black line and box. Each dot represents a FISH spot. n=167. FIG. 3C: Quantification of co-localized foci from MUC4-NR and MUC4-R GOLD FISH. The black numbers indicate spot number examined in each channel. FIG. 3D: (Top) A schematic showing 11 guide RNAs (MUC4-NR guide-RNA set 2) designed to target sites flanking the probes tiling region of MUC4-NR. (Bottom) A representative image of GOLD FISH using the MUC4-NR guide-RNA set 2 in an IMR-90 cell. Scale bar, 5 μm. FIG. 3E: (Left panel) Histogram of number of MUC4-NR foci detected in each cell using the MUC4-NR guide-RNA set 2 (n=76). Percentages of total cells are indicated. (Right panel) Box plot of signal-to-background ratio of MUC4-NR FISH spots using the MUC4-NR guide-RNA set 2. Mean±SD are represented using a black line and box. Each dot represents a FISH spot (n=90). FIG. 3F: (Top) A schematic showing MUC4-I1 guide RNA designed to target a repetitive region that is 30-kb away from the probes tiling region of MUC4-NR. (Bottom) A representative image of GOLD FISH using the MUC4-I1 guide RNA in an IMR-90 cell. Scale bar, 5 μm. FIG. 3G: Histogram of number of MUC4-NR foci detected in each cell using the MUC4-I1 guide RNA (n=70). Percentages of total cells are indicated.

FIG. 4 (includes FIGS. 4A-4E) shows GOLD FISH shows conformational differences of active ChrX and inactive ChrX by multi-color imaging and chromosomal scale paint. FIG. 4A: (Top) A schematic of TAD5 and TAD37 regions in chromosome X. (Bottom) A representative image of GOLD FISH against TAD5 (magenta) and TAD37 (green) in an IMR-90 cell. MacroH2A.1 immunostaining (cyan) was used to distinguish inactive ChrX from active ChrX. White arrow indicates the inactive ChrX. Scale bar, 5 μm. FIG. 4B: Box plots of distance between TAD5 and TAD37 of ChrX for active ChrX and inactive ChrX. Mean±SD are represented using a line and box. Each dot represents a ChrX measured (n=83 for active ChrX and 86 for inactive ChrX). ***P<0.001 (Student's t-test). FIG. 4C: DNA probe design for ChrX paint GOLD FISH. The primary probe has two Priming regions for PCR amplification, a Readout region complementary to fluorescently labeled Readout probe and an Encoding region for hybridization to genomic DNA. Cas9 RNP and Rep-X are omitted in this figure. FIG. 4D: A representative image of p-arm (green) and q-arm (magenta) of ChrX ‘painted’ by GOLD FISH. MacroH2A.1 immunostaining (cyan) was used to distinguish inactive ChrX from active ChrX. White arrow indicates the inactive ChrX. Scale bar, 5 μm. FIG. 4E: Box plots of center of mass distance between p-arm and q-arm of ChrX for active ChrX and inactive ChrX. Each dot represents a ChrX measured (n=101 for active ChrX and 99 for inactive ChrX). Mean±SD are represented using a line and box. ***P<0.001 (Student's t-test).

FIG. 5 (includes FIGS. 5A-5F) shows HER2 gene amplification detection in human tissue samples. FIG. 5A: A schematic of HER2 gene, CEP17 and RARA gene in chromosome 17 (Chr17). FIG. 5B: A representative view of GOLD FISH against HER2 gene (yellow) with HER2 protein immunostaining (red) and DNA staining by Hoechst 33342 (blue) on a breast cancer tissue sample from a patient. Sub-regions outlined in green boxes are zoomed showing HER2 amplified cells and HER2 non-amplified cells, respectively. Scale bar, 10 μm. FIG. 5C: A representative view of GOLD FISH against HER2 gene (yellow, left) and CEP17 (green, right) with HER2 protein immunostaining (red) and DNA staining by Hoechst 33342 (blue). Scale bar, 10 μm. FIG. 5D: Histograms of number of HER2 foci in each cell of the breast cancer tissue sample (n=161). Percentages of total cells are indicated. FIG. 5E: Histograms of number of CEP17 foci in each cell (n=161). Percentages of total cells are indicated. FIG. 5F: Box plots of loci number ratio of HER2 to CEP17 and HER2 to RARA. Each dot represents one cell. Mean±SD are represented using a line and box (n=159 for HER2/CEP17 and n=95 for HER2/RARA). ***P<0.001 (Student's t-test).

FIG. 6 (includes FIGS. 6A-6E) shows GOLD FISH against a repetitive region within the MUC4 gene (MUC4-R), related to FIG. 2. FIG. 6A: (Left) A representative image of GOLD FISH against MUC4-R using dCas9. Scale bar, 10 m. (Right) Quantification of detected loci number per cell for GOLD FISH against MUC4-R using dCas9 (n=55). Percentages of total cells are indicated. FIG. 6B: (Left) A representative image of GOLD FISH against MUC4-R using Cas9_{dHNH} in the absence of ATP. Scale bar, 10 m. (Right) Quantification of number of detected loci per cell for GOLD FISH against MUC4-R using Cas9_{dHNH} in the absence of ATP (n=41). Percentages of total cells are indicated. FIG. 6C: (Left) Schematic of CASFISH using ATTO550-labeled guide RNA against MUC4-R and Cas9_{dHNH}. (Right) A representative image of the CASFISH experiment. Scale bar, 5 m. FIG. 6D: (Left) Schematic of CASFISH using ATTO550-labeled guide RNA against MUC4-R and dCas9. (Right) A representative image of the CASFISH experiment. Scale bar, 5 m. FIG. 6E: (E) (Left) Nuclear background in ATTO550 channel after the co-localization assay against MUC4-R using ATTO550-labeled guide RNA and no Cas9 enzyme control. Each dot represents the average nuclear intensity in a cell. Mean±SD are represented using lines and boxes (n=51 for ‘+ Cas9’ and n=49 for ‘− Cas9’). (Right) Nuclear background in ATTO550 channel after CASFISH against MUC4-R using Cas9_{dHNH} with ATTO550-labeled guide RNA and no Cas9 enzyme control. Each dot represents the average nuclear intensity in a cell. Mean±SD are represented using lines and boxes (n=60 for ‘+ Cas9’ and n=54 for ‘− Cas9’). The nuclear background intensity in the ‘− Cas9’ control was negligible compared to that in ‘+ Cas9’, suggesting non-specific intracellular binding of ATTO550-labeled Cas9_{dHNH} RNP was mainly responsible to nuclear background in the co-localization assay and CASFISH.

FIG. 7 shows a representative imaging view of GOLD FISH against MUC4-NR in IMR-90 cells using MUC4-NR guide-RNA set 1, related to FIG. 3. Scale bar, 10 m.

FIG. 8 (includes FIGS. 8A-8E) shows results of buffered ethanol (BE70)-based fixation effectively preserved nuclear size, enabling GOLD FISH to demonstrate the conformational differences between active and inactive X chromosomes, related to FIG. 4. In this work we adopted two fixation methods: (1) MAA fixation (FIGS. 2 and 3): cells were fixed in MAA solution (pre-chilled methanol and acetic acid mixed at 1:1 ratio) for 20 min at −20° C. (2) BE70-MAA fixation (FIGS. 4 and 5): cells were fixed in BE70 at room temperature for 25 min, follow by incubation in MAA solution for 20 min at −20° C. We found the incubation in MAA solution was necessary for efficient GOLD FISH labeling in BE70-fixed cells, presumably because MAA solution further permeabilized the cells. See the ‘Fixation of cultured cell and tissue section’ section in ‘Method Details’ for details. FIG. 8A: Representative images of DNA stained by Hoechst 33342 in live and ‘after GOLD FISH’ IMR-90 cells using different fixatives. Scale bar, m. The ‘After GOLD FISH’ cells were fixed using either the first fixation method (MAA) or the second fixation method (BE70+MAA) described above, followed by a GOLD FISH protocol. See ‘Comparison of live and after-GOLD FISH cells’ section in ‘Method Details’ for details. FIG. 8B: Quantification of projected nuclear area change in live and ‘after GOLD FISH’ cells. AreaLive, the projected nuclear area in a live cell. AreaGOLDFISH, the projected nuclear area in the same cell after fixation (by either MAA or BE70+MAA) and GOLD FISH. The y-axis is the ratio of AreaGOLDFISH to AreaLive. Each dot represents one nucleus (n=43 for Buffered ethanol and n=38 for methanol-acetic acid). Mean±SD are represented using a black line and box. Buffered ethanol fixation preserves nuclear size substantially better than methanol-acetic acid fixation. See ‘Comparison of live and after-GOLD FISH cells’ section in ‘Method Details’ for details. ***P<0.001 (Student's t-test). FIG. 8C: A representative image of GOLD FISH against TAD5 (magenta) and TAD37 (green) regions in IMR-90 cells. MacroH2A.1 immunostaining (cyan) was performed to distinguish inactive ChrX from active ChrX. Scale bar, m. FIG. 8D: A representative image of GOLD FISH using TAD5 guide RNAs with TAD37 probes (green) and quantification of foci per cell (n=44). Percentages of total cells are indicated in the histogram. MacroH2A.1 immunostaining (cyan) was performed to distinguish inactive ChrX from active ChrX. Scale bar, 5 m. FIG. 8E: A representative image of GOLD FISH using TAD37 guide RNAs with TAD5 probes (magenta) in and quantification of foci per cell (n=55). Percentages of total cells are indicated in the histogram. MacroH2A.1 immunostaining (cyan) was performed to distinguish inactive ChrX from active ChrX. Scale bar, 5 m.

FIG. 9 (includes FIGS. 9A-9B) shows p-arm and q-arm ‘paint’ of ChrX by GOLD FISH, related to FIG. 4. FIG. 9A: A representative imaging view of p-arm (green) and q-arm (magenta) of ChrX ‘painted’ using GOLD FISH in IMR-90 cells. Scale bar, 10 m. FIG. 9B: Box plot of volumes of active ChrX and inactive ChrX using GOLD FISH. Each dot represents one ChrX. Mean±SD are represented using lines and boxes. (n=101 for active ChrX and n=99 for inactive ChrX). ***P<0.001 (Student's t-test).

FIG. 10 (includes FIGS. 10A-10D) shows GOLD FISH in tissue samples, crRNA synthesis for GOLD FISH, GOLD FISH probe density and GOLD FISH in PFA-fixed cells, related to FIG. 5. FIG. 10A: A representative view of GOLD FISH against HER2 gene (yellow, left) and RARA (cyan, right) with HER2 protein immunostaining (red) and DNA staining by Hoechst 33342 (blue) on a breast cancer tissue sample from a patient. Scale bar, 10 m. FIG. 10B: Transcription efficiency comparison of canonical crRNA and 5′-extended crRNA. (Left) The sequences of four canonical crRNAs (cr1, cr2, cr3, cr4), four 5′-extended crRNAs (Ex-cr1, Ex-cr2, Ex-cr3, Ex-cr4) and the DNA template for in vitro transcribing Ex-cr4. The protospacer sequences is bold-faced. The common 5′-extension sequence is colored in blue, which makes in vitro transcription efficiency of different crRNAs more homogenous. The DNA template for in vitro transcribing Ex-cr4 were partially double-stranded, including a double-stranded T7 promoter region and a single-stranded template region. (Right) A PAGE gel image shows in vitro transcription yields of different crRNAs under the same reaction condition. FIG. 10C: Probe densities of GOLD FISH, iFISH and OligoMiner against TAD37, TAD5, RARA, HER2 and MUC4-NR regions. Here the ‘probes’ in GOLD FISH refers to DNA oligo probes. ‘Balanced’, ‘Coverage’ and ‘Stringent’ are different probe mining parameters in the OligoMiner DNA FISH method. The probe densities of iFISH and OligoMiner against the five non-repetitive regions were obtained from ifish4u.org/probe-design/. FIG. 10D: A representative view of GOLD FISH against MUC4-NR using MUC4-NR guide-RNA set 1 in PFA-fixed IMR-90 cells. Nuclei were stained by Hoechest 33342 (blue). Scale bar, 10 μm.

FIG. 11 (includes FIGS. 11A-11D) shows single-nucleotide variation detection by GOLD FISH. GOLD FISH was performed to target a repetitive region (“MUC4-R”, green signals) and a non-repetitive region (“MUC4-SNV”, magenta signals) within the MUC4 gene using (A and B) on-target guide RNA or (C and D) 1 mismatched guide RNA against the MUC4-SNV region. (B and D) Histograms show number MUC4-SNV foci in each cell versus cell counts.

FIGS. 12A-12C show single-nucleotide variation detection by sgGOLDFISH. FIG. 12A: Schematic of SNV detection using sgGOLDFISH. Genomic DNA in red is homologous to guide RNA. FIG. 12B: Top, sequences of MUC4-NR target protospacer and gMUC4-OneMM or gMUC4-TwoMM. The blue-colored G represents the extended guanine at the 5′ of the guide RNA. Red-colored nucleotides represent mismatches. Bottom, a representative sgGOLDFISH image using gMUC4-OneMM (single cells outlined in orange are magnified on the upper-right corner), histograms of sgGOLDFISH foci using gMUC4-OneMM (n=78) or gMUC4-TwoMM (n=100), and quantification of co-localized foci from MUC4-R and MUC4-NR probes. FIG. 12C: Top, sequences of LMNA target protospacer and gLMNA-WT. Bottom, a representative sgGOLDFISH image using gLMNA-WT (single cells outlined in orange are magnified on the upper-right corner), and histograms of sgGOLDFISH foci using gLMNA-WT (n=121) or gLMNA-MUT (n=110).

FIGS. 13A-13I show SNV detection in HGPS cells. FIG. 13A: Schematic of HGPS pathogenic point mutation. FIG. 13B: Schematic of ABE editing of HGPS fibroblasts. c, Base identity at the HGPS mutation site before and after ABE treatment. The error bar represents mean±SD (n=3). FIGS. 13D, 13E: sgGOLDFISH in parallel with progerin immunofluorescence using (FIG. 13D) gLMNA-MUT or (FIG. 13E) gLMNA-WT. FIGS. 13F and 13G: Quantifications of progerin immunofluorescence intensity from different populations. Each dot represents a quantified cell (n=44 to 152 for each condition). Mann-Whitney U Test is used. n.s. represents p>0.05. Box represents the range of 25^{th} to 75^{th} percentiles, and whisker represents the range of 10^{th} to 90^{th} percentiles. Median line is shown in the box. FIG. 13H: Schematic of measuring distance from the FISH spot to the nuclear edge or the major/minor axes using sgGOLDFISH image data. FIG. 13I: Quantifications of the relative distance (i.e., the distance from a FISH spot to the nuclear edge divided the square root of the nuclear area) of LMNA and MUC4 alleles to the nuclear edge. Each dot represents a quantified FISH spot (n=538 to 994 for each condition). Student's t test is used. Median line is shown. Whisker represents mean±SD.

FIGS. 14A, 14B show a comparison between GOLDFISH and sgGOLDFISH. FIG. 14A: Schematic of GOLDFISH. The Cas9 nickase RNP is applied to fixed and permeabilized cells to cleave the genomic DNA. Then Rep-X along with ATP is added to unwind the genomic DNA from the Cas9 cleavage sites. Finally, fluorescently labeled oligo FISH probes are added to hybrid to sequences of interest. Multiple different guide RNA species and oligo FISH probes are used in the GOLDFISH. The target region (i.e., guide RNA target protospacers and probe hybridization sites) spans typically 2 kb to 5 kb. FIG. 14B: Schematic of sgGOLDFISH. The experimental procedure is the same as GOLDFISH, but only 1 guide RNA species is used in the sgGOLDFISH. The target region (i.e., the guide RNA target protospacer and probe hybridization sites) spans typically around 1.5 kb.

FIGS. 15A-15E show the eCas9 RNP and the in vitro cleavage assay. FIG. 15A: Schematic of eCas9 RNP. Compared to canonical guide RNA, the 5′ extended guide RNA used in this study has an extra guanine (bolded in the figure) at the 5′ of the crRNA. FIG. 15B: Schematic of in vitro cleavage assay. eCas9 RNP was mixed with DNA substrate and incubated for 1 hour at 37° C. Then proteinase K was added to digest bound and free eCas9. Finally, the reaction was loaded into an agarose gel for electrophoresis. FIG. 15C: Sequences of DNA substrate and guide RNA tested in the in vitro cleavage assay. The DNA substrate was PCR-synthesized using human genomic DNA and primers against a non-repetitive region of the MUC4 gene. A group of guide RNAs with 1 or 2 mismatches against the target protospacer were used in the cleavage assay. The blue “G” represents the 5′ extended guanine of the crRNA. The red colored nucleotides represent mismatches against the DNA substrate. FIG. 15D: Gel image of the in vitro cleavage assay using guide RNA with 5′ extended guanine. FIG. 15E: Gel image of the in vitro cleavage assay using canonical guide RNA (i.e., without the 5′ extended guanine). Significant cleavage activity was observed with the canonical guide RNA even if there are two mismatches between the guide RNA and DNA substrate.

FIGS. 16A-16C show the SSB-ddPCR assay. FIG. 16A: Schematic of SSB-ddPCR. FIG. 16B: Representative SSB-ddPCR results using eCas9 nickase and (top) gMUC4-TwoMM or (bottom) gMUC4-OneMM. FIG. 16C: Left, bar plot of fraction of −FAM+HEX droplets from the SSB-ddPCR using gMUC4-TwoMM or gMUC4-OneMM. Right, standard curve of the ddPCR assay. Student's t test is used. n.s. represents p>0.05. Error bar represents mean standard deviation from at least 3 replicates.

FIGS. 17A-17C show the control experiments for SSB-ddPCR. FIG. 17A: The in vitro cleavage assay to measure the efficiency of DNA cleavage by Cas9 nickase RNP in the step 2 in FIG. 16A. In this assay, 600 ng PCR-synthesized DNA substrate (FIG. 15C) was mixed with 400 nM Cas9 nickase RNP cleaving the top strand and 400 nM Cas9 nickase RNP cleaving the bottom strand, and incubated for 1 hour at 37° C. The 400 nM Cas9 RNP cleaving the bottom strand was also used in the step 2 in FIG. 16A. Less than 600 ng genomic DNA was harvested in the step 1 in FIG. 16A. After proteinase K treatment, the reaction was heated at 90° C. for 1 min to dissociate the two parts of the double-nicked DNA, followed by agarose gel electrophoresis. FIG. 17B: Gel image of the in vitro cleavage assay. Only the 3^{rd} lane shows close to cleavage efficiency indicates the 400 nM Cas9 RNP cleaving the bottom strand cleaved almost all DNA molecules. FIG. 17C: Representative SSB-ddPCR result using dCas9 and gMUC4-OneMM.

FIG. 18 shows the generation of the standard curve of SSB-ddPCR. Schematic of the generating standard curve in FIG. 16C.

FIGS. 19A, 19B show a schematic of sgGOLDFISH against the MUC4-NR region and GOLDFISH against the MUC4-R region. FIG. 19A: Only one guide RNA (gMUC4-OneMM or gMUC4-TwoMM) and 23 oligo FISH probes are used to target the MUC4-NR region. The figure shows the case that gMUC4-OneMM is used (there is one mismatch between guide RNA and target protospacer). In contrast, although only one guide RNA (gMUC4-R) and one oligo FISH probe are used to target MUC4-R region, the MUC4-R region contains multiple repeats, therefore multiple binding sites for eCas9 nickase RNP complexed with gMUC4-R and the FISH probe against MUC4-R region. FIG. 19B: Top, sequences of MUC4-NR target protospacer and gMUC4-OneMM or gMUC4-TwoMM. The blue-colored G represents the extended guanine at the 5′ of the guide RNA. Red-colored nucleotides represent mismatches. Bottom, a representative sgGOLDFISH image using gMUC4-OneMM without proteinase treatment in HEK293T cells (single cells outlined in orange are magnified on the upper-right corner), and histograms of sgGOLDFISH foci using gMUC4-OneMM (n=109) or gMUC4-TwoMM (n=94).

FIGS. 20A, 20B show an in vitro cleavage assay to measure cleavage activity of eCas9 in complex with different guide RNA against the LMNA gene. FIG. 20A: The DNA substrate was PCR-synthesized using human genomic DNA and primers against a non-repetitive region of the LMNA gene. A group of guide RNAs with PAM-distal mismatches against the target protospacer were used in the cleavage assay. The blue “G” represents the 5′ extended guanine of the crRNA. The red colored nucleotides represent mismatches against the DNA substrate. FIG. 20B: Gel image of the in vitro cleavage assay using the guide RNAs and the DNA substrate in FIG. 20A.

FIGS. 21A-21D show schematics of sgGOLDFISH against LMNA. FIG. 21A: Schematic of sgGOLDFISH against LMNA using gLMNA-MUT or gLMNA-WT. The figure shows the scenario that gLMNA-WT is used to target a wild-type LMNA allele (there is one mismatch between guide RNA and target protospacer). FIG. 21B: Statistics of the width of the LMNA foci from the sgGOLDFISH using gLMNA-WT in FIG. 12C. Each dot represents a quantified LMNA focus (n=137). Median line is shown. Whisker represents mean±standard deviation (SD). FIG. 21C: Sequences of the target protospacer of the LMNA-WT allele and gLMNA-MUT or gLMNA-WT. The blue “G” represents the 5′ extended guanine of the guide RNA. The red colored nucleotides represent mismatches against the protospacer. FIG. 21D: Sequences of the target protospacer of the LMNA-MUT allele and gLMNA-MUT or gLMNA-WT. The blue “G” represents the 5′ extended guanine of the guide RNA. The red colored nucleotides represent mismatches against the protospacer. The bolded ‘A-T’ base pair indicates the LMNA c.1824 C>T mutation in HGPS fibroblasts.

FIGS. 22A-22D show the DNA-free base editing to correct the HGPS pathogenic point mutation. FIG. 22A: Representative Sanger sequencing results of untreated and ABE-treated HGPS fibroblasts. The black arrow indicates the HGPS pathogenic point mutation site. FIG. 22B: Representative images showing whisker represents the morphology difference of Lamin A/C meshwork between untreated and ABE-treated HGPS fibroblasts. White arrows indicate morphologically abnormal nuclei. FIG. 22C: Quantification of fraction of morphologically abnormal nuclei in untreated and ABE-treated HGPS fibroblasts. Morphologically abnormal nuclei were identified by visual inspection. More than 150 cells were quantified for each condition. The dataset was quantified twice by two persons independently. The error bar indicates the difference between the two quantifications. Student's t test is used. FIG. 22D: Quantifications of base identity at the HGPS pathogenic point mutation site at different time points after mixing untreated and ABE-treated HGPS fibroblasts at 1:1 ratio (i.e., 1:1 mixture). The base identity was measure by Sanger sequencing. The data indicates the 1:1 mixture contains roughly 50% uncorrected and 50% ABE-corrected HGPS fibroblast within 24 hours.

FIGS. 23A-23C show that sgGOLDFISH signals can be used for spatial analysis. FIG. 23A: A representative image of sgGOLDFISH against LMNA using gLMNA-MUT and progerin immunofluorescence in 1:1 mixture. The “mutant-positive cells” are indicated by white arrows. FIG. 23B: A representative image of sgGOLDFISH against LMNA using gLMNA-WT and progerin immunofluorescence in 1:1 mixture. The “correction-positive cell” is indicated by a white arrow. FIG. 23C: Lamin A/C-ChIP data of the HGPS fibroblasts from a previous study (https://research.nhgri.nih.gov/manuscripts/Collins/HGPSepigenetics)²⁰. The enrichment (i.e., Avg Log(HGPS/Input)) of the LMNA and MUC4 genes in Lamin A/C ChIP is shown. Smaller value (i.e., more negative) suggests less enriched in Lamin A/C ChIP, which shows the LMNA gene is less enriched than the MUC4 gene in Lamin A/C ChIP. This is consistent with our data in FIG. 23B which shows that the MUC4 alleles are closer the nuclear edge than the LMNA alleles.

### DETAILED DESCRIPTION

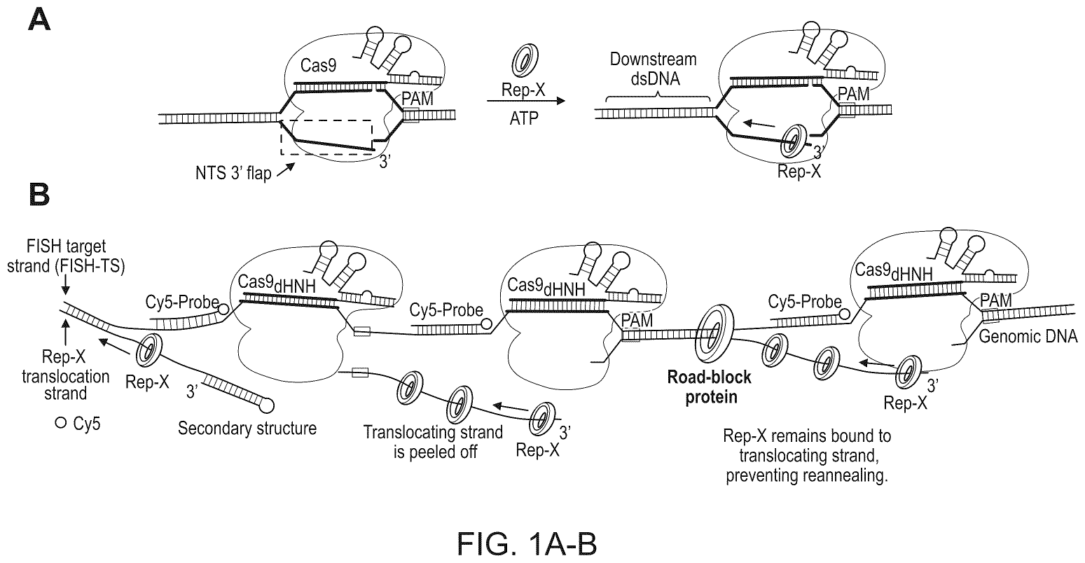

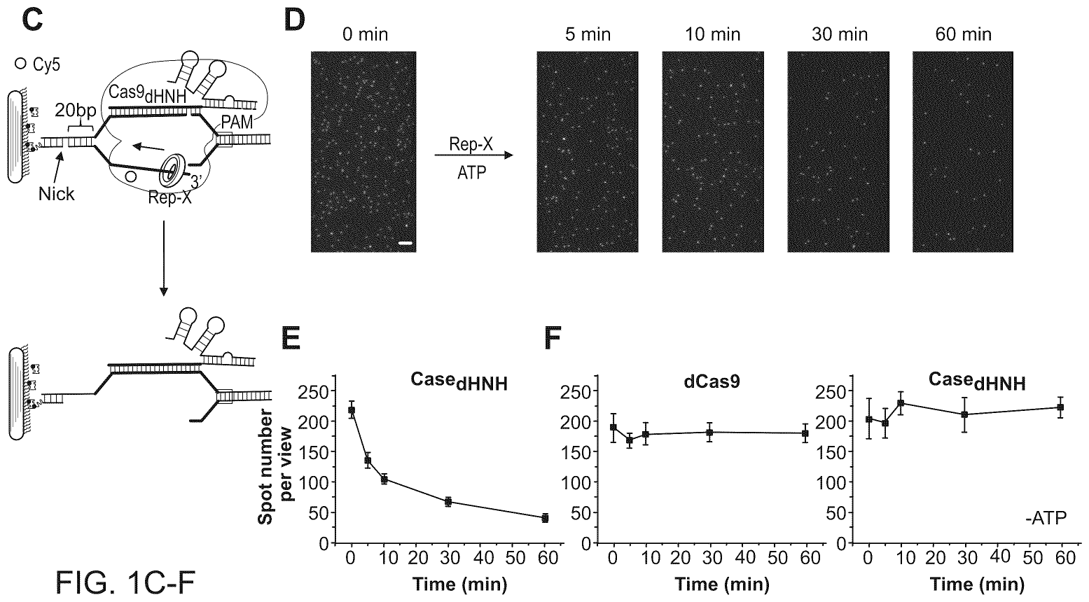

We developed Genome Oligopaint via Local Denaturation Fluorescence in Situ Hybridization (GOLD FISH). Our systems are clear improvements over DNA FISH technology. See FIG. 1B.

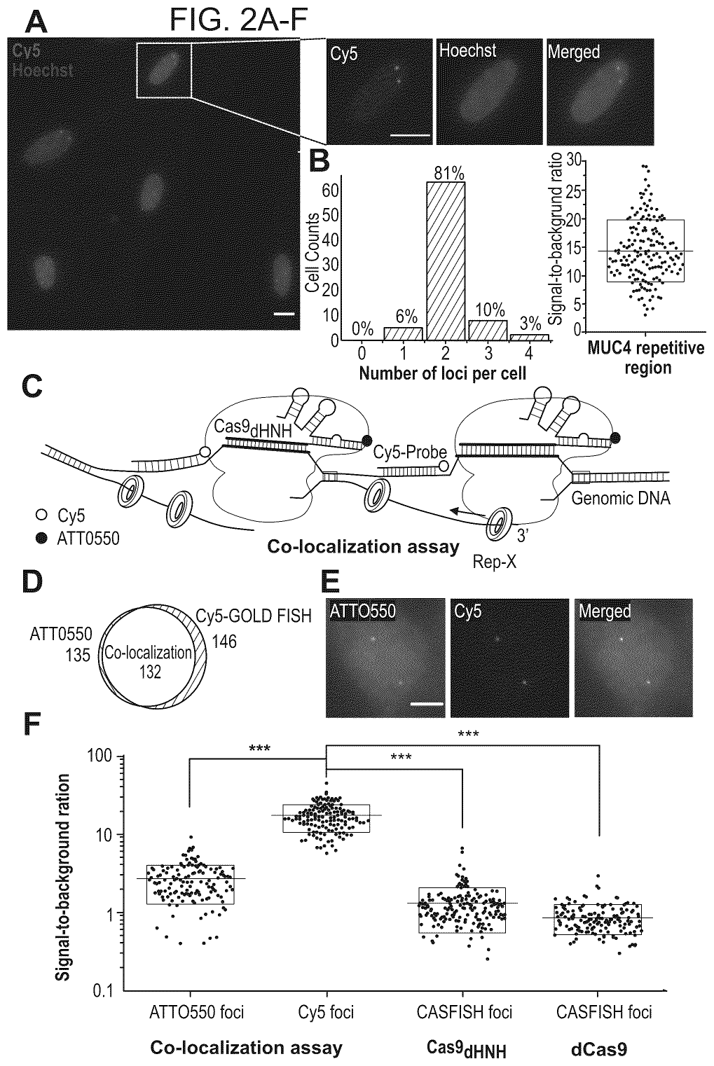

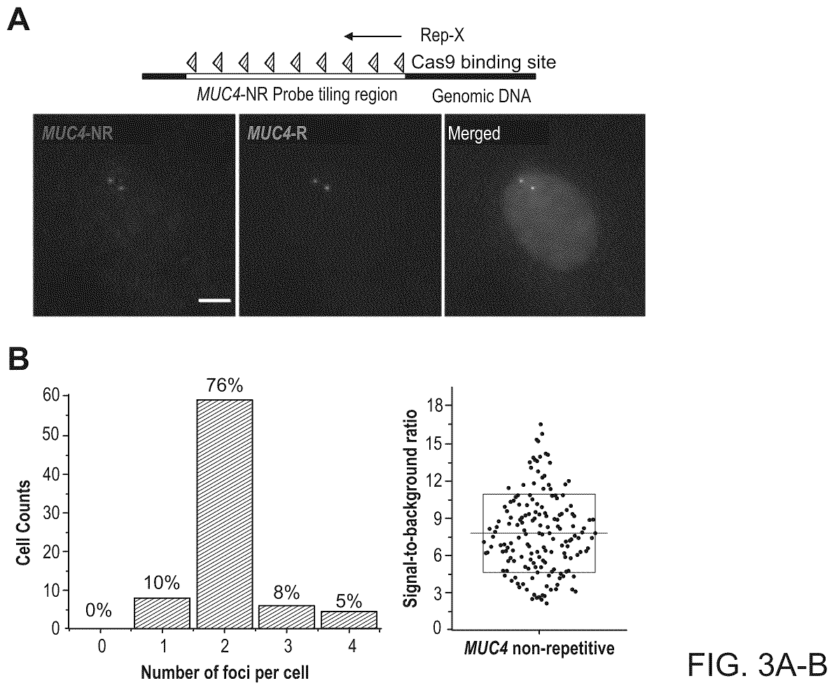

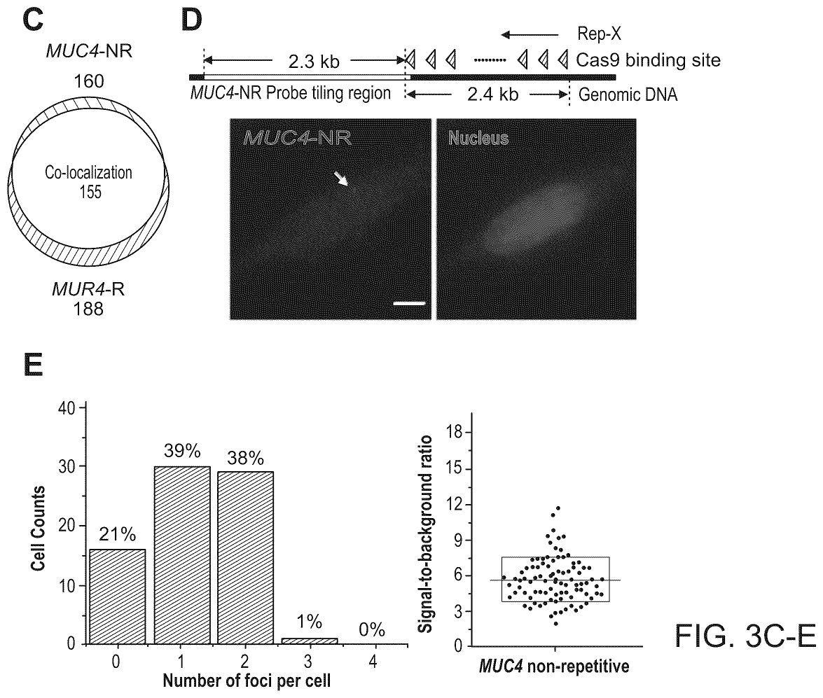

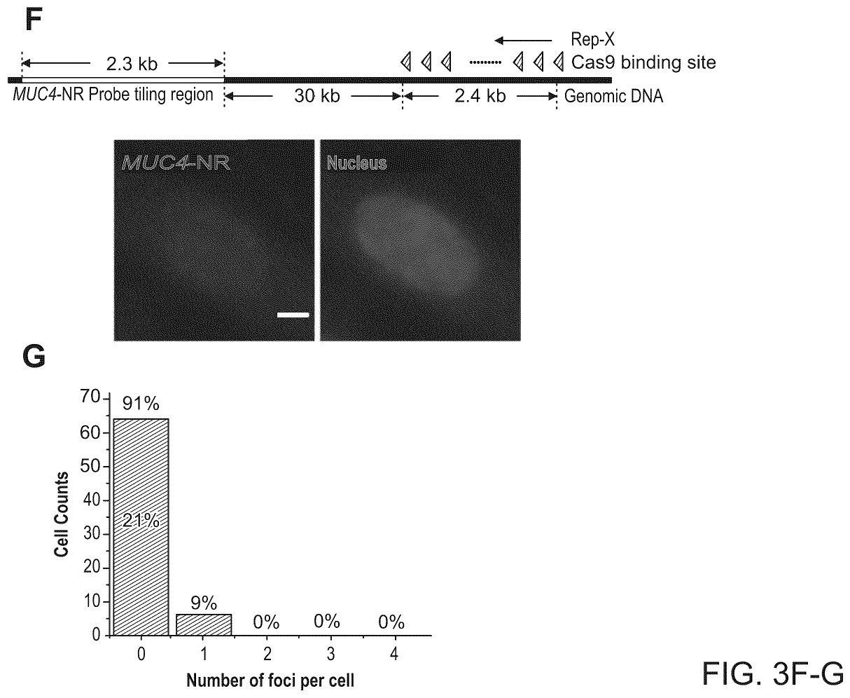

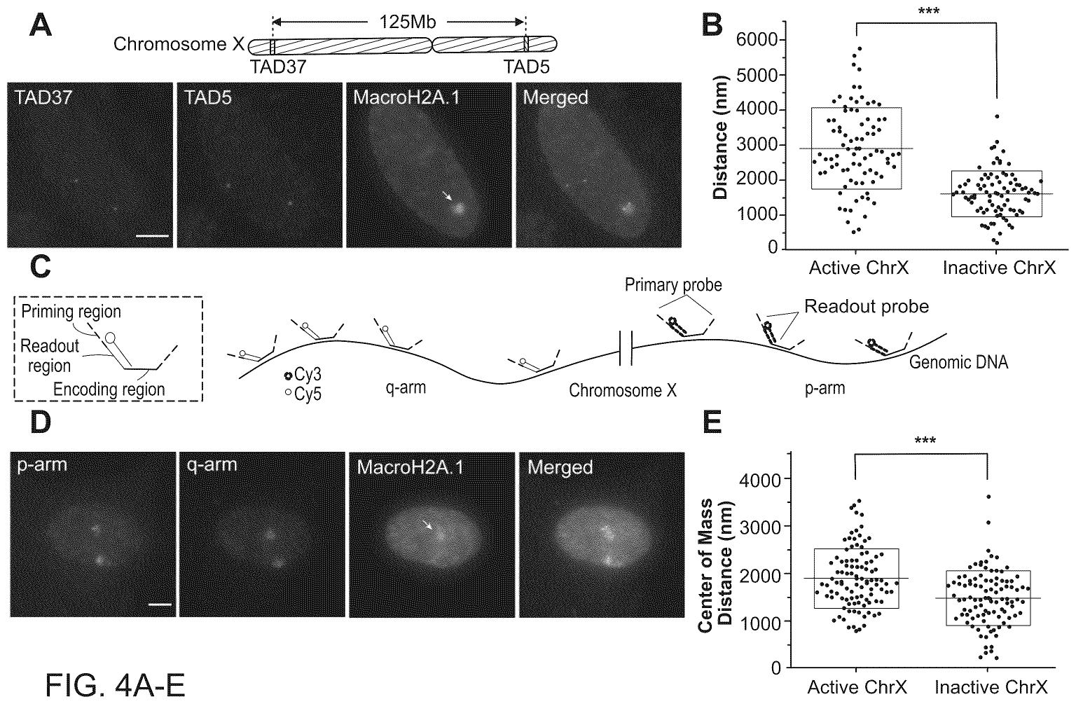

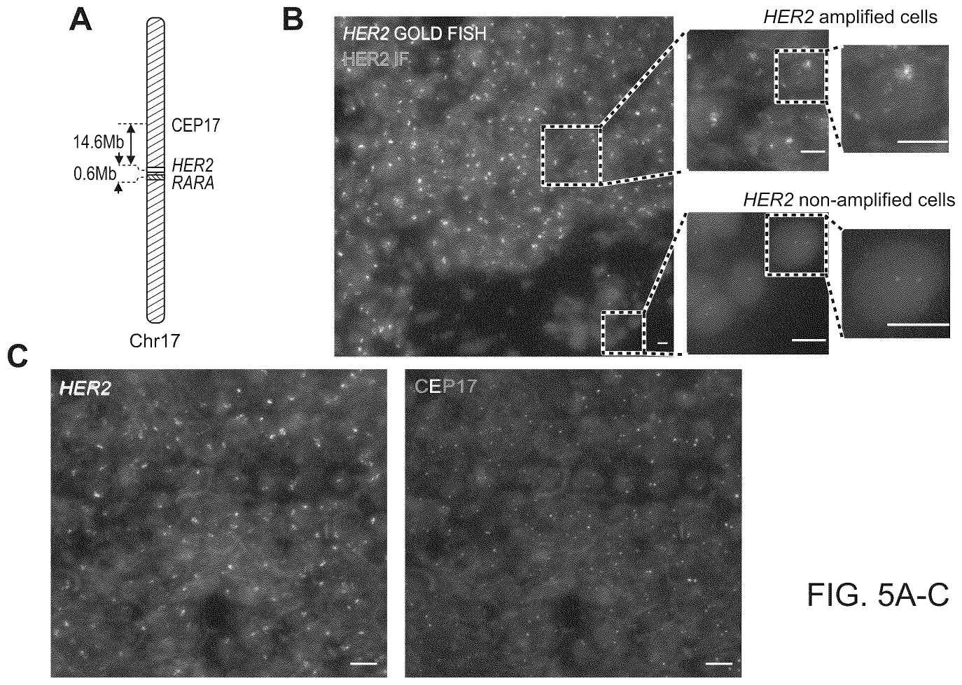

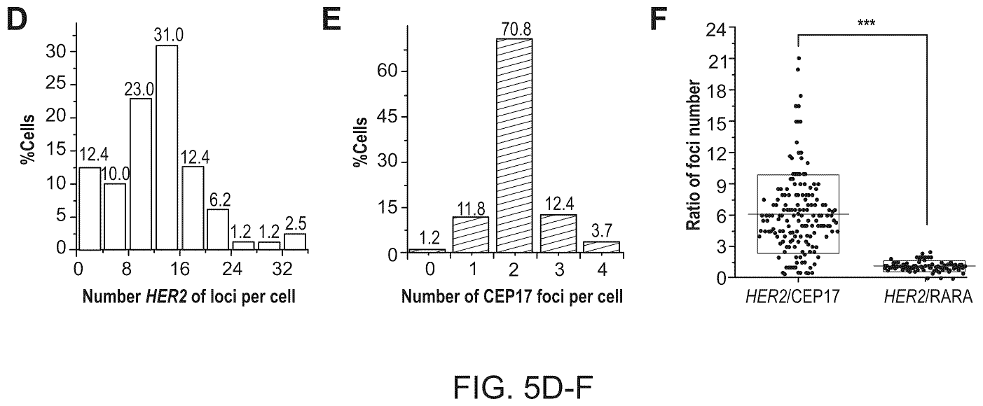

Compared to traditional DNA FISH which requires global denaturation of genomic DNA using harsh denaturing conditions, GOLD FISH locally denatures genomic DNA by programmed loading of an engineered superhelicase at nicks generated by the CRISPR-Cas9 nickase, allowing fluorescently labeled probes to hybridize with sequences of interest (See FIGS. 1A-1B). GOLD FISH labels target genomic loci with much higher signal-to-background ratio compared to other CRISPR-Cas9 based genome imaging methods such as CASFISH (See FIG. 2). GOLD FISH robustly targets non-repetitive genomic DNA sequences ranging for example from 2 kilobases to whole chromosome, which can be used for imaging of chromatin conformations in basic research (See FIG. 3 and FIG. 4). Traditional DNA FISH requires days to detect HER2 gene amplification in potential breast cancer patient tissue. In contrast, GOLD FISH can rapidly detect HER2 gene amplification in patient tissue samples within 6 hours, potentially facilitating clinical diagnosis (See FIG. 5).

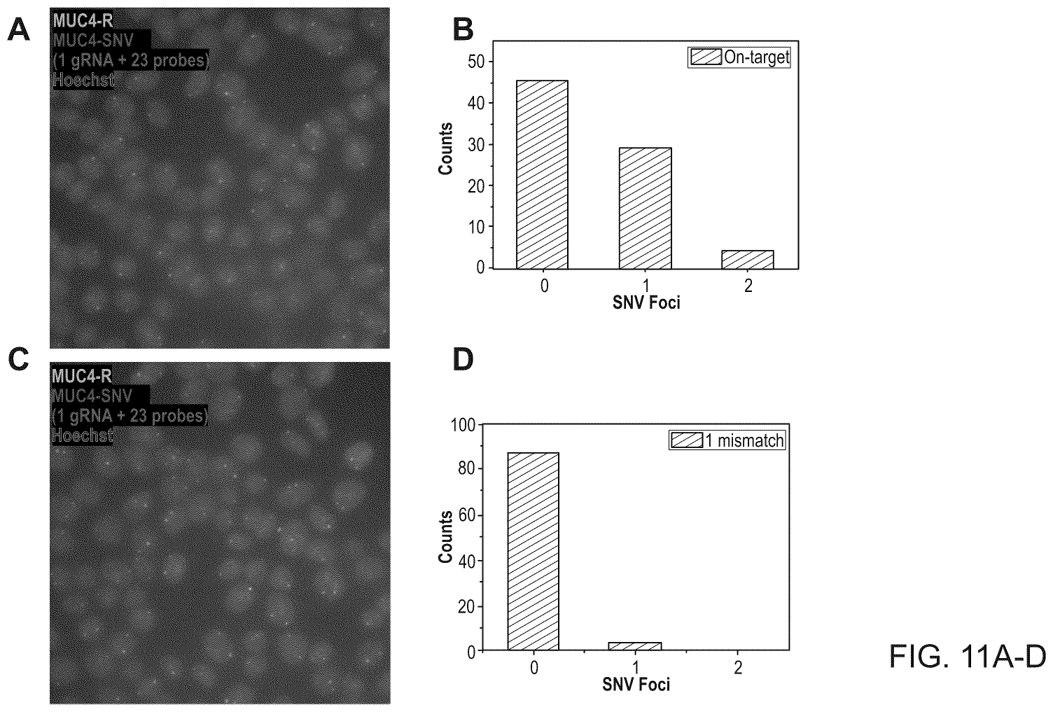

One of unique feature of GOLD FISH is that the labeling of GOLD FISH relies on Cas9 nicking the genomic DNA strand, which provide a 3′ single-stranded DNA overhang, allowing Rep-X to load on and unwind downstream dsDNA (See FIGS. 1A-1B). Cas9's DNA nicking activity can be fine-tuned by intentionally introducing mismatches into guide-RNA in combination with previously engineered high-specificity Cas9 variants, so that any additional single-nucleotide mismatch against guide RNA will inhibit nicking of target genomic DNA. By using the nicking activity fine-tuned Cas9 RNP, GOLD FISH can achieve single-nucleotide sensitivity (See FIG. 11). In FIG. 11, GOLD FISH was performed to target a repetitive region (“MUC4-R”, green signals) and a non-repetitive region (“MUC4-SNV”, magenta signals) within the MUC4 gene. When on-target guide RNA against the MUC4-SNV region was used, ˜50% cells had at least one MUC4-SNV foci (See FIGS. 11A and 11B). However, when a guide RNA with 1 nucleotide mismatch against desired sequence was used, the labeling efficiency was minimal (See FIGS. 11C and 11D). The data suggest that GOLD FISH can detect single-nucleotide mutations at single-cell level. Many diseases are cause by a single nucleotide mutation. For example, the Hutchinson-Gilford progeria syndrome (characterized by accelerated ageing) is caused by a point mutation (c. 1824 C>T) in the LMNA gene. GOLD FISH could be used to image cells carrying the single-nucleotide mutations of interest in patient tissue samples. In addition, base editors become more and more popular genome editing tools because they do not generate double-strand breaks during editing. GOLD FISH can in situ label cells that have been edited by base editors, provide additional spatial information at single-cell level compared to other sequencing-based methods.

Accordingly, in some embodiments, are methods and compositions comprising a CRISPR-associated (Cas) peptide or a nucleic acid sequence encoding the CRISPR-associated (Cas) peptide and a plurality of guide nucleic acids or a nucleic acid sequence encoding the plurality of guide nucleic acids. In some embodiments, compositions and methods described herein comprise 1, 2, 3, 4, 5, 6, or more than 6 gRNAs. In some embodiments, compositions and methods described herein comprise 1, 2, 3, 4, 5, 6, or more than 6 different gRNAs. In some embodiments, compositions and methods described herein comprise 4 or at least 4 different gRNAs.

In certain embodiments, the compositions of the disclosure include nucleic acids encoding gene editing agents and at least one guide RNA (gRNA) that is complementary to a target sequence, such as for example, a tumor nucleic acid sequence, a virus sequence, a genetic disorder and the like. Indeed, the target sequence can be any sequence wherein a mutation may be present. In certain embodiments, the gene editing agents comprise: Cre recombinases, CRISPR/Cas molecules, TALE transcriptional activators, Cas9 nucleases, nickases, transcriptional regulators, homologues, orthologs or combinations thereof. In certain embodiments, the target sequences comprise coding sequences, noncoding sequences or combinations thereof. In certain embodiments, the guide nucleic acid sequences target one or more target sequences comprising: structural gene sequences, enzymatic gene sequences, regulatory genes, and the like.

In one embodiment, a composition comprises a viral vector encoding a gene editing agent and at least one guide RNA (gRNA) wherein the gRNA is complementary to a target nucleic acid sequence of a virus gene sequence, a tumor gene sequence, a mutation in a disease or disorder, (e.g. sickle cell anemia) or any target sequence that the user may want to investigate and determine one or more mutations in the target sequence. In certain embodiments, a target nucleic acid sequence is in a coding region. In certain embodiments, the target sequence is in a non-coding sequence.

In certain embodiments, the gene editing agents comprise: Cre recombinases, CRISPR/Cas molecules, TALE transcriptional activators, Cas9 nucleases, nickases, transcriptional regulators, homologues, orthologs or combinations thereof. In one embodiment, the gene editing agent is a Clustered Regularly Interspaced Short Palindromic Repeated (CRISPR)-associated endonuclease, or homologues or orthologs thereof. In another embodiment, the CRISPR-associated endonuclease is Cas9 or homologues or orthologs, thereof.

In certain embodiments, the gene editing agent is a Clustered Regularly Interspaced Short Palindromic Repeated (CRISPR)-associated endonuclease, or homologues thereof. An example of a CRISPR-associated endonuclease is Cas9 or homologues or orthologs thereof.

In some embodiments, different gRNAs target different sequences within a target nucleic acid sequence. In some embodiments, the different gRNAs are complementary to different target sequences within a target gene. In some embodiments, a target sequence is within or near a target gene. In some embodiments, a region near a target gene comprises 1, 2, 3, 4.5, 10, 15, 20, 25, 30, or 35 base positions surrounding the target gene.

In some embodiments, a first guide nucleic acid of a plurality of guide nucleic acids is complementary to a first target sequence in or surrounding a target gene. In some embodiments, a second guide nucleic acid of the plurality of guide nucleic acids is complementary to a second target sequence in or surrounding a target gene. In some embodiments, a third guide nucleic acid of the plurality of guide nucleic acid is complementary to a third target sequence in or surrounding a target gene. In some embodiments, a fourth guide nucleic acid of the plurality of guide nucleic acid is complementary to a fourth target sequence in or surrounding a target gene. In some embodiments, the first target sequence, the second target sequence, the third target sequence, and the fourth target sequence are different.

In some embodiments, compositions and methods described herein comprise 2, 3, 4, 5, 6, or more than 6 different gRNAs that target (e.g., hybridize or anneal to) or are complementary to a region within or surrounding a target nucleic acid sequence.

In some embodiments, compositions and methods described herein comprise 2, 3, 4, 5, 6, or more than 6 different gRNAs that target a region within or surrounding a first target gene. In some embodiments, compositions and methods described herein comprise 2, 3, 4, 5, 6, or more than 6 different gRNAs that target a region within or surrounding a second target gene. In some embodiments, compositions and methods described herein comprise 2, 3, 4, 5, 6, or more than 6 different gRNAs that target a region within or surrounding a third target gene. In some embodiments, compositions and methods described herein comprise 2, 3, 4, 5, 6, or more than 6 different gRNAs that target a region within or surrounding a fourth target gene. In some embodiments, compositions and methods described herein comprise 2, 3, 4, 5, 6, or more than 6 different gRNAs that target a region within or surrounding a fifth, sixth or more target genes.

In some embodiments, a gRNA target sequence comprises a sequence at least or about 70%, 80%, 85%, 90%, 91%, 92%, 93%, 94%, 95%, 96%, 97%, 98%, 99%, or 100% sequence identity to a target nucleic acid sequence. In some embodiments, a gRNA target sequence comprises a sequence at least or about 70%, 80%, 85%, 90%, 91%, 92%, 93%, 94%, 95%, 96%, 97%, 98%, 99%, or 100% sequence identity to a sequence complementary to a target nucleic acid sequence. In some instances, a gRNA target sequence comprises a sequence at least or about 95% homology to a target nucleic acid sequence. In some instances, a gRNA target sequence comprises a sequence at least or about 95% homology to a sequence complementary to a target nucleic acid sequence. In some instances, a gRNA target sequence comprises a sequence at least or about 97% homology to a target nucleic acid sequence. In some instances, a gRNA target sequence comprises a sequence at least or about 97% homology to a sequence complementary to a target nucleic acid sequence. In some instances, a gRNA target sequence comprises a sequence at least or about 99% homology to a target nucleic acid sequence. In some instances, a gRNA target sequence comprises a sequence at least or about 99% homology to a sequence complementary to a target nucleic acid sequence. In some instances, a gRNA target sequence comprises a sequence at least or about 100% homology to a target nucleic acid sequence. In some instances, a gRNA target sequence comprises a sequence at least or about 100% homology to a sequence complementary to a target nucleic acid sequence.

In some embodiments, a gRNA target sequence comprises a sequence at least or about 70%, 80%, 85%, 90%, 91%, 92%, 93%, 94%, 95%, 96%, 97%, 98%, 99%, or 100% sequence identity to a target nucleic acid sequence in Tables 1, 2 or 4. In some embodiments, a gRNA target sequence comprises a sequence at least or about 70%, 80%, 85%, 90%, 91%, 92%, 93%, 94%, 95%, 96%, 97%, 98%, 99%, or 100% sequence identity to a sequence complementary to a target nucleic acid sequence in Tables 1, 2 or 4.

In certain embodiments, a viral vector comprises an adenovirus vector, an adeno-associated viral vector (AAV), or derivatives thereof. In some embodiments, the nucleic acids are configured to be packaged into an adeno-associated virus (AAV) vector. In some embodiments, the adeno-associated virus (AAV) vector is AAV2, AAV5, AAV6, AAV7, AAV8, or AAV9. In some embodiments, the adeno-associated virus (AAV) vector is AAV1, AAV2, AAV3, AAV4, AAV5, AAV6, AAV7, AAV8, AAV9, AAV10, AAV11, AAVDJ, or AAVDJ/8.

In another embodiment, an expression vector comprises an isolated nucleic acid encoding a gene editing agent and at least one guide RNA (gRNA) wherein the gRNA is complementary to a target nucleic acid sequence. In embodiments, the gene editing agents comprise: Cre recombinases, CRISPR/Cas molecules, TALE transcriptional activators, Cas9 nucleases, nickases, transcriptional regulators, homologues, orthologs or combinations thereof. In one embodiment, the gene editing agent is a Clustered Regularly Interspaced Short Palindromic Repeated (CRISPR)-associated endonuclease, or homologues or orthologs thereof. In another embodiment, the CRISPR-associated endonuclease is Cas9 or homologues or orthologs, thereof.

In some embodiments, the expression vector encodes a transactivating small RNA (tracrRNA) wherein the transactivating small RNA (tracrRNA) sequence is fused to the sequence encoding the guide RNA.

In another embodiment, the expression vector further comprises a sequence encoding a nuclear localization signal.

In some embodiments, the CRISPR-endonuclease is a Cas9 endonuclease, a Cas12 endonuclease, a CasX endonuclease, or a CasΦ endonuclease. In some embodiments, the CRISPR-endonuclease is a Cas9 nuclease. In some embodiments, the Cas9 nuclease is a Staphylococcus aureus Cas9 nuclease.

### Gene Editing Agents

Compositions of the disclosure include at least one gene editing agent, comprising CRISPR-associated nucleases such as Cas9 and Cas12a gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof.

In recent years, several systems for targeting endogenous genes have been developed including homing endonucleases (HE) or meganucleases, zinc finger nucleases (ZFN), transcription activator-like effector nucleases (TALEN) and most recently clustered regularly interspaced short palindromic repeats (CRISPR)-associated system 9 (Cas9) proteins which utilize site-specific double-strand DNA break (DSB)-mediated DNA repair mechanisms. These enzymes induce a precise and efficient genome cutting through DSB-mediated DNS repair mechanisms. These DSB-mediated genome editing techniques enable target gene deletion, insertion, or modification.

In the past years, ZFNs and TALENs have revolutionized genome editing. The major drawbacks for ZFNs and TALENs are the uncontrollable off-target effects and the tedious and expensive engineering of custom DNA-binding fusion protein for each target site, which limit the universal application and clinical safety.

CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al., Annu Rev Genet (2011) 45:273-297; Wiedenheft et al., Nature (2012) 482:331-338); Jinek M et al., Science (2012) 337:816-821; Cong L et al., Science (2013) 339:819-823; Jinek M et al., (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

In general, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains. The mutation can comprise one or more deletions. The mutation can comprise one or more point mutations, that is, the replacement of a single nucleotide with another nucleotide. Useful point mutations are those that have functional consequences, for example, mutations that result in the conversion of an amino acid codon into a termination codon, or that result in the production of a nonfunctional protein.

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

The RNA-guided Cas9 biotechnology induces genome editing without detectable off-target effects. This technique takes advantage of the genome defense mechanisms in bacteria that CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). Cas9 belongs to the type II CRISPR/Cas system and has strong endonuclease activity to cut target DNA.

In certain embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In one embodiment, the RNA-guided endonuclease is derived from a type II CRISPR/Cas system. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately. Therefore, the Cas9 gRNA technology requires the expression of the Cas9 protein and gRNA, which then form a gene editing complex at the specific target DNA binding site within the target genome and inflict cleavage/mutation of the target DNA.

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cas12a (also known as Cpf1) to edit the target site of interest.

Engineered CRISPR systems generally contain two components: a guide RNA (gRNA or sgRNA) and a CRISPR-associated endonuclease (Cas protein). In nature, CRISPR/CRISPR-associated (Cas) systems provide bacteria and archaea with adaptive immunity against viruses and plasmids by using CRISPR RNAs (crRNAs) to guide the silencing of invading nucleic acids.

The CRISPR-Cas is a RNA-mediated adaptive defense system that relies on small RNA molecules for sequence-specific detection and silencing of foreign nucleic acids. CRISPR/Cas systems are composed of cas genes organized in operon(s) and CRISPR array(s) consisting of genome-targeting sequences (called spacers).

As described herein, CRISPR-Cas systems generally refer to an enzyme system that includes a guide RNA sequence that contains a nucleotide sequence complementary or substantially complementary to a region of a target polynucleotide, and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. In certain embodiments, CRISPR-Cas systems contain engineered and/or mutated Cas proteins. In some embodiments, nucleases generally refer to enzymes capable of cleaving the phosphodiester bonds between the nucleotide subunits of nucleic acids. In some embodiments, endonucleases are generally capable of cleaving the phosphodiester bond within a polynucleotide chain. Nickases refer to endonucleases that cleave only a single strand of a DNA duplex.

In some embodiments, the CRISPR/Cas system used herein can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. By way of further example, in some embodiments, the CRISPR-Cas protein is a Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/CasΦ, Cas12L etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas12. In certain embodiments, the Cas12 polypeptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is CasΦ.

In some embodiments, the Cas9 protein can be from or derived from: Staphylococcus aureus, Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Fine goldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the composition comprises a CRISPR-associated (Cas) protein, or functional fragment or derivative thereof. In some embodiments, the Cas protein is an endonuclease, including but not limited to the Cas9 nuclease. In some embodiments, the Cas9 protein comprises an amino acid sequence identical to the wild type Streptococcus pyogenes or Staphylococcus aureus Cas9 amino acid sequence. In some embodiments, the Cas protein comprises the amino acid sequence of a Cas protein from other species, for example other Streptococcus species, such as thermophilus; Pseudomonas aeruginosa, Escherichia coli, or other sequenced bacteria genomes and archaea, or other prokaryotic microorganisms. Other Cas proteins, useful for the present disclosure, known or can be identified, using methods known in the art (see e.g., Esvelt et al., 2013, Nature Methods, 10: 1116-1121). In some embodiments, the Cas protein comprises a modified amino acid sequence, as compared to its natural source. CRISPR/Cas proteins comprise at least one RNA recognition and/or RNA binding domain. RNA recognition and/or RNA binding domains interact with guide RNAs (gRNAs). CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the Cas protein. The CRISPR/Cas-like protein can also be truncated or modified to optimize the activity of the effector domain of the Cas protein.

In some embodiments, the CRISPR/Cas-like protein can be derived from a wild type Cas protein or fragment thereof. In some embodiments, the CRISPR/Cas-like protein is a modified Cas9 protein. For example, the amino acid sequence of the Cas9 protein can be modified to alter one or more properties (e.g., nuclease activity, affinity, stability, etc.) of the protein relative to wild-type or another Cas protein. Alternatively, domains of the Cas9 protein not involved in RNA-guided cleavage can be eliminated from the protein such that the modified Cas9 protein is smaller than the wild-type Cas9 protein.

The disclosed CRISPR-Cas compositions should also be construed to include any form of a protein having substantial homology to a Cas protein (e.g., Cas9, saCas9, Cas9 protein) disclosed herein. In some embodiments, a protein which is “substantially homologous” is about 50% homologous, about 70% homologous, about 80% homologous, about 90% homologous, about 95% homologous, or about 99% homologous to amino acid sequence of a Cas protein disclosed herein. The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In some embodiments, the composition comprises a CRISPR-associated (Cas) peptide, or functional fragment or derivative thereof. In certain embodiments, the Cas peptide is an endonuclease, including but not limited to the Cas9 nuclease. In some embodiments, the Cas9 peptide comprises an amino acid sequence identical to the wild type Streptococcus pyogenes Cas9 amino acid sequence. In some embodiments, the Cas peptide may comprise the amino acid sequence of a Cas protein from other species, for example other Streptococcus species, such as thermophilus; Pseudomonas aeruginosa, Escherichia coli, or other sequenced bacteria genomes and archaea, or other prokaryotic microorganisms. Other Cas peptides, useful for the present disclosure, known or can be identified, using methods known in the art (see e.g., Esvelt et al., 2013, Nature Methods, 10: 1116-1121). In certain embodiments, the Cas peptide may comprise a modified amino acid sequence, as compared to its natural source. For example, in some embodiments, the wild type Streptococcus pyogenes Cas9 sequence can be modified. In certain embodiments, the amino acid sequence can be codon optimized for efficient expression in human cells (i.e., “humanized) or in a species of interest. A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1 GL669193761; or KM099233.1 GL669193765 or Cas9 amino acid sequence of PX330 or PX260 (Addgene, Cambridge, MA).

The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution).

In certain embodiments, the Cas peptide is a mutant Cas9, wherein the mutant Cas9 reduces the off-target effects, as compared to wild-type Cas9. In some embodiments, the mutant Cas9 is a Streptococcus pyogenes Cas9 (SpCas9) variant.

In some embodiments, SpCas9 variants comprise one or more point mutations, including, but not limited to R780A, K810A, K848A, K855A, H982A, K1003A, and R1060A (Slaymaker et al., 2016, Science, 351(6268): 84-88). In some embodiments, SpCas9 variants comprise D1135E point mutation (Kleinstiver et al., 2015, Nature, 523(7561): 481-485). In some embodiments, SpCas9 variants comprise one or more point mutations, including, but not limited to N497A, R661A, Q695A, Q926A, D1135E, L169A, and Y450A (Kleinstiver et al., 2016, Nature, doi:10.1038/nature16526). In some embodiments, SpCas9 variants comprise one or more point mutations, including but not limited to M495A, M694A, and M698A. Y450 is involved with hydrophobic base pair stacking. N497, R661, Q695, Q926 are involved with residue to base hydrogen bonding contributing to off-target effects. N497 hydrogen bonding through peptide backbone. L169A is involved with hydrophobic base pair stacking. M495A, M694A, and H698A are involved with hydrophobic base pair stacking.

In some embodiments, SpCas9 variants comprise one or more point mutations at one or more of the following residues: R780, K810, K848, K855, H982, K1003, R1060, D1135, N497, R661, Q695, Q926, L169, Y450, M495, M694, and M698. In some embodiments, SpCas9 variants comprise one or more point mutations selected from the group of: R780A, K810A, K848A, K855A, H982A, K1003A, R1060A, D1135E, N497A, R661A, Q695A, Q926A, L169A, Y450A, M495A, M694A, and M698A.

In some embodiments, the SpCas9 variant comprises the point mutations, relative to wildtype SpCas9, of N497A, R661A, Q695A, and Q926A. In some embodiments, the SpCas9 variant comprises the point mutations, relative to wildtype SpCas9, of N497A, R661A, Q695A, Q926A, and D1135E. In some embodiments, the SpCas9 variant comprises the point mutations, relative to wildtype SpCas9, of N497A, R661A, Q695A, Q926A, and L169A. In some embodiments, the SpCas9 variant comprises the point mutations, relative to wildtype SpCas9, of N497A, R661A, Q695A, Q926A, and Y450A. In some embodiments, the SpCas9 variant comprises the point mutations, relative to wildtype SpCas9, of N497A, R661A, Q695A, Q926A, and M495A. In some embodiments, the SpCas9 variant comprises the point mutations, relative to wildtype SpCas9, of N497A, R661A, Q695A, Q926A, and M694A. In some embodiments, the SpCas9 variant comprises the point mutations, relative to wildtype SpCas9, of N497A, R661A, Q695A, Q926A, and H698A. In some embodiments, the SpCas9 variant comprises the point mutations, relative to wildtype SpCas9, of N497A, R661A, Q695A, Q926A, D1135E, and L169A. In some embodiments, the SpCas9 variant comprises the point mutations, relative to wildtype SpCas9, of N497A, R661A, Q695A, Q926A, D1135E, and Y450A. In some embodiments, the SpCas9 variant comprises the point mutations, relative to wildtype SpCas9, of N497A, R661A, Q695A, Q926A, D1135E, and M495A. In some embodiments, the SpCas9 variant comprises the point mutations, relative to wildtype SpCas9, of N497A, R661A, Q695A, Q926A, D1135E, and M694A. In some embodiments, the SpCas9 variant comprises the point mutations, relative to wildtype SpCas9, of N497A, R661A, Q695A, Q926A, D1135E, and M698A.

In some embodiments, the SpCas9 variant comprises the point mutations, relative to wildtype SpCas9, of R661A, Q695A, and Q926A. In some embodiments, the SpCas9 variant comprises the point mutations, relative to wildtype SpCas9, of R661A, Q695A, Q926A, and D1135E. In some embodiments, the SpCas9 variant comprises the point mutations, relative to wildtype SpCas9, of R661A, Q695A, Q926A, and L169A. In some embodiments, the SpCas9 variant comprises the point mutations, relative to wildtype SpCas9, of R661A, Q695A, Q926A, and Y450A. In some embodiments, the SpCas9 variant comprises the point mutations, relative to wildtype SpCas9, of R661A, Q695A, Q926A, and M495A. In some embodiments, the SpCas9 variant comprises the point mutations, relative to wildtype SpCas9, of R661A, Q695A, Q926A, and M694A. In some embodiments, the SpCas9 variant comprises the point mutations, relative to wildtype SpCas9, of R661A, Q695A, Q926A, and H698A. In some embodiments, the SpCas9 variant comprises the point mutations, relative to wildtype SpCas9, of R661A, Q695A, Q926A, D1135E, and L169A. In some embodiments, the SpCas9 variant comprises the point mutations, relative to wildtype SpCas9, of R661A, Q695A, Q926A, D1135E, and Y450A. In some embodiments, the SpCas9 variant comprises the point mutations, relative to wildtype SpCas9, of R661A, Q695A, Q926A, D1135E, and M495A. In some embodiments, the SpCas9 variant comprises the point mutations, relative to wildtype SpCas9, of R661A, Q695A, Q926A, D1135E, and M694A. In some embodiments, the SpCas9 variant comprises the point mutations, relative to wildtype SpCas9, of R661A, Q695A, Q926A, D1135E, and M698A.

In some embodiments, the mutant Cas9 comprises one or more mutations that alter PAM specificity (Kleinstiver et al., 2015, Nature, 523(7561):481-485; Kleinstiver et al., 2015, Nat Biotechnol, 33(12): 1293-1298). In some embodiments, the mutant Cas9 comprises one or more mutations that alter the catalytic activity of Cas9, including but not limited to D10A in RuvC and H840A in HNH (Cong et al., 2013; Science 339: 919-823, Gasiubas et al., 2012; PNAS 109:E2579-2586 Jinek et al., 2012; Science 337: 816-821).

In addition to the wild type and variant Cas9 endonucleases described, embodiments of the disclosure also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9(K848A/K1003A/R1060A) (a.k.a. eSPCas9 1.1) are employed in the compositions. The disclosure is by no means limited to these variants, and also encompasses all Cas9 variants (Slaymaker, I. M. et al. (2015)). The present disclosure also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

Accordingly, in certain embodiments, a Cas9 variant comprises a human-optimized Cas9; a nickase mutant Cas9; saCas9; enhanced-fidelity SaCas9 (efSaCas9); SpCas9(K855a); SpCas9(K810A/K1003A/r1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A.

As used herein, the term “Cas” is meant to include all Cas molecules comprising variants, mutants, orthologues, high-fidelity variants and the like.

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cpf1 to edit the target site of interest. Cpf1 is a single crRNA-guided, class 2 CRISPR effector protein which can effectively edit target DNA sequences in human cells. Exemplary Cpf1 includes, but is not limited to, Acidaminococcus sp. Cpf1 (AsCpf1) and Lachnospiraceae bacterium Cpf1 (LbCpf1).

The disclosure should also be construed to include any form of a peptide having substantial homology to a Cas peptide (e.g., Cas9) disclosed herein. Preferably, a peptide which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of a Cas peptide disclosed herein.

The peptide may alternatively be made by recombinant means or by cleavage from a longer polypeptide. The composition of a peptide may be confirmed by amino acid analysis or sequencing.

The variants of the peptides according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present disclosure, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the disclosure can be post-translationally modified. For example, post-translational modifications that fall within the scope of the present disclosure include signal peptide cleavage, glycosylation, acetylation, isoprenylation, proteolysis, myristylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the disclosure may include unnatural amino acids formed by post-translational modification or by introducing unnatural amino acids during translation. A variety of approaches are available for introducing unnatural amino acids during protein translation.

A peptide or protein of the disclosure may be conjugated with other molecules, such as proteins, to prepare fusion proteins. This may be accomplished, for example, by the synthesis of N-terminal or C-terminal fusion proteins provided that the resulting fusion protein retains the functionality of the Cas peptide.

A peptide or protein of the disclosure may be phosphorylated using conventional methods such as the method described in Reedijk et al. (The EMBO Journal 11(4):1365, 1992).

Cyclic derivatives of the peptides of the disclosure are also part of the present disclosure. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the disclosure, cyclic peptides may comprise a beta-turn in the right position. Beta-turns may be introduced into the peptides of the disclosure by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The disclosure also relates to peptides comprising a Cas peptide fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e. are heterologous).

In some embodiments, the targeting domain can be a membrane spanning domain, a membrane binding domain, or a sequence directing the protein to associate with for example vesicles or with the nucleus. In some embodiments, the targeting domain can target a peptide to a particular cell type or tissue. For example, the targeting domain can be a cell surface ligand or an antibody against cell surface antigens of a target tissue (e.g. cancerous tissue). A targeting domain may target the peptide of the disclosure to a cellular component. In certain embodiments, the targeting domain targets a tumor-specific antigen or tumor-associated antigen.

N-terminal or C-terminal fusion proteins comprising a peptide or chimeric protein of the disclosure conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the Cas peptide or chimeric protein fused to the selected protein or marker protein as described herein. Examples of proteins which may be used to prepare fusion proteins include immunoglobulins, glutathione-S-transferase (GST), hemagglutinin (HA), and truncated myc.

A peptide of the disclosure may be synthesized by conventional techniques. For example, the peptides of the disclosure may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, supra, Vol 1, for classical solution synthesis.).

A peptide of the disclosure may be prepared by standard chemical or biological means of peptide synthesis. Biological methods include, without limitation, expression of a nucleic acid encoding a peptide in a host cell or in an in vitro translation system.

Biological preparation of a peptide of the disclosure involves expression of a nucleic acid encoding a desired peptide. An expression cassette comprising such a coding sequence may be used to produce a desired peptide. For example, subclones of a nucleic acid sequence encoding a peptide of the disclosure can be produced using conventional molecular genetic manipulation for subcloning gene fragments, such as described by Sambrook et al., Molecular Cloning: A Laboratory Manual, Cold Springs Laboratory, Cold Springs Harbor, New York (2012), and Ausubel et al. (ed.), Current Protocols in Molecular Biology, John Wiley & Sons (New York, NY) (1999 and preceding editions), each of which is hereby incorporated by reference in its entirety. The subclones then are expressed in vitro or in vivo in bacterial cells to yield a smaller protein or polypeptide that can be tested for a particular activity.

In the context of an expression vector, the vector can be readily introduced into a host cell, e.g., mammalian, bacterial, yeast or insect cell by any method in the art. Coding sequences for a desired peptide of the disclosure may be codon optimized based on the codon usage of the intended host cell in order to improve expression efficiency as demonstrated herein. Codon usage patterns can be found in the literature (Nakamura et al., 2000, Nuc Acids Res. 28:292). Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The expression vector can be transferred into a host cell by physical, biological or chemical means, discussed in detail elsewhere herein.

To ensure that the peptide obtained from either chemical or biological synthetic techniques is the desired peptide, analysis of the peptide composition can be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

The peptides and chimeric proteins of the disclosure may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In certain embodiments, a gene editing system comprises meganucleases. In some embodiments, the gene editing system comprises zinc finger nucleases (ZFNs). In some embodiments, the gene editing system comprises transcription activator-like effector nucleases (TALENs). These gene editing systems can be broadly classified into two categories based on their mode of DNA recognition: ZFNs, TALENs and meganucleases achieve specific DNA binding via protein-DNA interactions, whereas CRISPR-Cas systems are targeted to specific DNA sequences by a short RNA guide molecule that base-pairs directly with the target DNA and by protein-DNA interactions.

### Guide Nucleic Acids

The compositions of the disclosure include sequence encoding a guide RNA (gRNA) comprising a sequence that is complementary to a target sequence.

In some embodiments, the composition comprises at least one isolated guide nucleic acid, or fragment thereof, where the guide nucleic acid comprises a nucleotide sequence that is complementary to one or more target sequences. In some embodiments, the guide nucleic acid is a guide RNA (gRNA).

In some embodiments, the gRNA comprises a crRNA:tracrRNA duplex. In some embodiments, the gRNA comprises a stem-loop that mimics the natural duplex between the crRNA and tracrRNA. In some embodiments, the stem-loop comprises a nucleotide sequence comprising AGAAAU. For example in some embodiments, the composition comprises a synthetic or chimeric guide RNA comprising a crRNA, stem, and tracrRNA.

In certain embodiments, the composition comprises an isolated crRNA and/or an isolated tracrRNA which hybridize to form a natural duplex. For example, in some embodiments, the gRNA comprises a crRNA or crRNA precursor (pre-crRNA) comprising a targeting sequence.

In some embodiments, the gRNA comprises a nucleotide sequence that is substantially complementary to a target sequence. The target sequence may be any sequence in any coding or non-coding region.

The guide RNA sequence can be a sense or anti-sense sequence. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neisseria meningitidis requires 5′-NNNNGATT). The specific sequence of the guide RNA may vary, but, regardless of the sequence, useful guide RNA sequences will be those that minimize off-target effects while achieving high efficiency. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude off-target human transcriptome or (even rarely) untranslated-genomic sites.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

When the compositions are administered in an expression vector, the guide RNAs can be encoded by a single vector. Alternatively, multiple vectors can be engineered to each include two or more different guide RNAs. When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the guide RNA sequences or in a separate vector.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-di methylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶N-methyladenosine; N⁶,N⁶-dimethyladenosine; N⁶,2′-O-trimethyladenosine; 2-methylthio-N⁶N-isopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N²,N²-dimethyl guanosine; N²,2′-O-dimethyl guanosine; N²,N²,2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

In certain embodiments, the composition comprises multiple different gRNAs, each targeted to a different target sequence. In certain embodiments, this multiplexed strategy provides for increased efficacy. In some embodiments, the compositions described herein utilize about 1 gRNA to about 6 gRNAs. In some embodiments, the compositions described herein utilize at least about 1 gRNA. In some embodiments, the compositions described herein utilize at most about 6 gRNAs. In some embodiments, the compositions described herein utilize about 1 gRNA to about 2 gRNAs, about 1 gRNA to about 3 gRNAs, about 1 gRNA to about 4 gRNAs, about 1 gRNA to about 5 gRNAs, about 1 gRNA to about 6 gRNAs, about 2 gRNAs to about 3 gRNAs, about 2 gRNAs to about 4 gRNAs, about 2 gRNAs to about 5 gRNAs, about 2 gRNAs to about 6 gRNAs, about 3 gRNAs to about 4 gRNAs, about 3 gRNAs to about 5 gRNAs, about 3 gRNAs to about 6 gRNAs, about 4 gRNAs to about 5 gRNAs, about 4 gRNAs to about 6 gRNAs, or about 5 gRNAs to about 6 gRNAs. In some embodiments, the compositions described herein utilize about 1 gRNA, about 2 gRNAs, about 3 gRNAs, about 4 gRNAs, about 5 gRNAs, or about 6 gRNAs.

In some embodiments, the gRNA is a synthetic oligonucleotide. In some embodiments, the synthetic nucleotide comprises a modified nucleotide. Modification of the inter-nucleoside linker (i.e. backbone) can be utilized to increase stability or pharmacodynamic properties. For example, inter-nucleoside linker modifications prevent or reduce degradation by cellular nucleases, thus increasing the pharmacokinetics and bioavailability of the gRNA. Generally, a modified inter-nucleoside linker includes any linker other than other than phosphodiester (PO) liners, that covalently couples two nucleosides together. In some embodiments, the modified inter-nucleoside linker increases the nuclease resistance of the gRNA compared to a phosphodiester linker. For naturally occurring oligonucleotides, the inter-nucleoside linker includes phosphate groups creating a phosphodiester bond between adjacent nucleosides. In some embodiments, the gRNA comprises one or more inter-nucleoside linkers modified from the natural phosphodiester. In some embodiments all of the inter-nucleoside linkers of the gRNA, or contiguous nucleotide sequence thereof, are modified. For example, in some embodiments the inter-nucleoside linkage comprises sulfur (S), such as a phosphorothioate inter-nucleoside linkage.

Modifications to the ribose sugar or nucleobase can also be utilized herein. Generally, a modified nucleoside includes the introduction of one or more modifications of the sugar moiety or the nucleobase moiety. In some embodiments, the gRNAs, as described, comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or stability. Such modifications include those where the ribose ring structure is modified. These modifications include replacement with a hexose ring (HNA), a bicyclic ring having a biradical bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or —OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. By way of further example, in some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the gRNA comprises one or more modified sugars. In some embodiments, the gRNA comprises only modified sugars. In certain embodiments, the gRNA comprises greater than 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the gRNA comprises both inter-nucleoside linker modifications and nucleoside modifications.

Target specificity can be used in reference to a guide RNA, or a crRNA specific to a target polynucleotide sequence or region and further includes a sequence of nucleotides capable of selectively annealing/hybridizing to a target (sequence or region) of a target polynucleotide (e.g. corresponding to a target), e.g., a target DNA. In some embodiments, a crRNA or the derivative thereof contains a target-specific nucleotide region complementary to a region of the target DNA sequence. In some embodiments, a crRNA or the derivative thereof contains other nucleotide sequences besides a target-specific nucleotide region. In some embodiments, the other nucleotide sequences are from a tracrRNA sequence.

gRNAs are generally supported by a scaffold, wherein a scaffold refers to the portions of gRNA or crRNA molecules comprising sequences which are substantially identical or are highly conserved across natural biological species (e.g. not conferring target specificity). Scaffolds include the tracrRNA segment and the portion of the crRNA segment other than the polynucleotide-targeting guide sequence at or near the 5′ end of the crRNA segment, excluding any unnatural portions comprising sequences not conserved in native crRNAs and tracrRNAs. In some embodiments, the crRNA or tracrRNA comprises a modified sequence. In certain embodiments, the crRNA or tracrRNA comprises at least 1, 2, 3, 4, 5, 10, or 15 modified bases (e.g. a modified native base sequence).

Complementary, as used herein, generally refers to a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In some embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation. Hybridization generally refers to process in which two single-stranded polynucleotides bind non-covalently to form a stable double-stranded polynucleotide. A resulting double-stranded polynucleotide is a “hybrid” or “duplex.” In certain instances, 100% sequence identity is not required for hybridization and, in certain embodiments, hybridization occurs at about greater than 70%, 75%, 80%, 85%, 90%, or 95% sequence identity. In certain embodiments, sequence identity includes in addition to non-identical nucleobases, sequences comprising insertions and/or deletions.

The nucleic acid of the disclosure, including the RNA (e.g., crRNA, tracrRNA, gRNA) or nucleic acids encoding the RNA, may be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Dveksler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The isolated nucleic acids also can be chemically synthesized, either as a single nucleic acid (e.g., using automated DNA synthesis in the 3′ to 5′ direction using phosphoramidite technology) or as a series of oligonucleotides. Isolated nucleic acids of the disclosure also can be obtained by mutagenesis of, e.g., a naturally occurring portion crRNA, tracrRNA, RNA-encoding DNA, or of a Cas9-encoding DNA

In certain embodiments, the isolated RNAs are synthesized from an expression vector encoding the RNA molecule, as described in detail elsewhere herein.

### Nucleic Acids and Vectors

In some embodiments, the composition of the disclosure comprises an isolated nucleic acid encoding one or more elements of the CRISPR-Cas system described herein. For example, in some embodiments, the composition comprises an isolated nucleic acid encoding at least one guide nucleic acid (e.g., gRNA). In some embodiments, the composition comprises an isolated nucleic acid encoding a Cas peptide, or functional fragment or derivative thereof. In some embodiments, the composition comprises an isolated nucleic acid encoding at least one guide nucleic acid (e.g., gRNA) and encoding a Cas peptide, or functional fragment or derivative thereof. In some embodiments, the composition comprises an isolated nucleic acid encoding at least one guide nucleic acid (e.g., gRNA) and further comprises an isolated nucleic acid encoding a Cas peptide, or functional fragment or derivative thereof.

In some embodiments, the composition comprises at least one isolated nucleic acid encoding a gRNA, where the gRNA is substantially complementary to a target sequence. In some embodiments, the composition comprises at least one isolated nucleic acid encoding a gRNA, where the gRNA is complementary to a target sequence having at least 75%, 80%, 85%, 90%, 95%, 96%, 97%, 98%, or 99% sequence homology to a target sequence described herein.

In some embodiments, the composition comprises at least one isolated nucleic acid encoding a Cas peptide described elsewhere herein, or a functional fragment or derivative thereof. In some embodiments, the composition comprises at least one isolated nucleic acid encoding a Cas peptide having at least 75%, 80%, 85%, 90%, 95%, 96%, 97%, 98%, or 99% amino acid sequence homology with a Cas peptide described elsewhere herein.

The isolated nucleic acid may comprise any type of nucleic acid, including, but not limited to DNA and RNA. For example, in some embodiments, the composition comprises an isolated DNA, including for example, an isolated cDNA, encoding a gRNA or peptide of the disclosure, or functional fragment thereof. In some embodiments, the composition comprises an isolated RNA encoding a peptide of the disclosure, or a functional fragment thereof. The isolated nucleic acids may be synthesized using any method known in the art.

The present disclosure can comprise use of a vector in which the isolated nucleic acid described herein is inserted. The art is replete with suitable vectors that are useful in the present disclosure. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors is known in the art and is generally available.

In brief summary, the expression of natural or synthetic nucleic acids encoding an RNA and/or peptide is typically achieved by operably linking a nucleic acid encoding the RNA and/or peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the disclosure provides a gene therapy vector.

The isolated nucleic acid of the disclosure can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art.

In some embodiments, lentivirus vectors are used. For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In some embodiments, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

A variety of different AAV capsids have been described and can be used, although AAV which preferentially target the liver and/or deliver genes with high efficiency are particularly desired. The sequences of the AAV8 are available from a variety of databases. While the examples utilize AAV vectors having the same capsid, the capsid of the gene editing vector and the AAV targeting vector are the same AAV capsid. Another suitable AAV is, e.g., rh10 (WO 2003/042397). Still other AAV sources include, e.g., AAV9 (see, for example, U.S. Pat. No. 7,906,111; US 2011-0236353-A1), and/or hu37 (see, e.g., U.S. Pat. No. 7,906,111; US 2011-0236353-A1), AAV1, AAV2, AAV3, AAV4, AAV5, AAV6, AAV6.2, AAV7, AAV8, (U.S. Pat. Nos. 7,790,449; 7,282,199, WO 2003/042397; WO 2005/033321, WO 2006/110689; U.S. Pat. Nos. 7,790,449; 7,282,199; 7,588,772). Still other AAV can be selected, optionally taking into consideration tissue preferences of the selected AAV capsid.

In some embodiments, AAV vectors disclosed herein include a nucleic acid encoding a CRISPR-Cas systems described herein. In some embodiments, the nucleic acid also includes one or more regulatory sequences allowing expression and, in some embodiments, secretion of the protein of interest, such as e.g., a promoter, enhancer, polyadenylation signal, an internal ribosome entry site (“IRES”), a sequence encoding a protein transduction domain (“PTD”), and the like. Thus, in some embodiments, the nucleic acid comprises a promoter region operably linked to the coding sequence to cause or improve expression of the protein of interest in infected cells. Such a promoter can be ubiquitous, cell- or tissue-specific, strong, weak, regulated, chimeric, etc., for example, to allow efficient and stable production of the protein in the infected tissue. In certain embodiments, the promoter is homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters. In certain embodiments. other promoters used include promoters that are tissue specific for tissues such as kidney, spleen, and pancreas. Examples of ubiquitous promoters include viral promoters, particularly the CMV promoter, the RSV promoter, the SV40 promoter, etc., and cellular promoters such as the phosphoglycerate kinase (PGK) promoter and the b-actin promoter.

In some embodiments, the recombinant AAV vector comprises packaged within an AAV capsid, a nucleic acid, generally containing a 5′ AAV ITR, the expression cassettes described herein and a 3′ AAV ITR. As described herein, in some embodiments, an expression cassette contains regulatory elements for an open reading frame(s) within each expression cassette and the nucleic acid optionally contains additional regulatory elements.

The AAV vector, in some embodiments, comprises a full-length AAV 5′ inverted terminal repeat (ITR) and a full-length 3′ ITR. A shortened version of the 5′ ITR, termed AITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription (see, for example, D M McCarty et al, “Self-complementary recombinant adeno-associated virus (scAAV) vectors promote efficient transduction independently of DNA synthesis”, Gene Therapy, (August 2001); see also, for example, U.S. Pat. Nos. 6,596,535; 7,125,717; and 7,456,683). Where a pseudotyped AAV is to be produced, the ITRs are selected from a source which differs from the AAV source of the capsid. For example, in some embodiments, AAV2 ITRs are selected for use with an AAV capsid having a particular efficiency for a selected cellular receptor, target tissue or viral target. In some embodiments, the ITR sequences from AAV2, or the deleted version thereof (ΔITR), are used for convenience and to accelerate regulatory approval (i.e. pseudotyped). In some embodiments, a single-stranded AAV viral vector is used.

Methods for generating and isolating AAV viral vectors suitable for delivery to a subject are known in the art (see, for example, U.S. Pat. Nos. 7,790,449; 7,282,199; WO 2003/042397; WO 2005/033321, WO 2006/110689; and U.S. Pat. No. 7,588,772 B2, U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065). In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

### EXAMPLES

The following examples are illustrative.

### Example 1: Genome Oligopaint Via Local Denaturation Fluorescence In Situ Hybridization (GOLD FISH)

The CRISPR-Cas9 system from Streptococcus pyogenes has been widely used for genome editing in cells (Cong et al., 2013; Doudna and Charpentier, 2014; Mali et al., 2013b). In the CRISPR-Cas9 system, the Cas9 endonuclease can be programed with a guide RNA to target a desired DNA sequence (Gasiunas et al., 2012; Jinek et al., 2012; Sapranauskas et al., 2011). An on-target DNA substrate of Cas9 ribonucleoprotein (RNP) contains a 20-nucleotide (nt) protospacer region complementary to the spacer sequence of guide RNA, and a protospacer adjacent motif (PAM, 5′-NGG-3′ for Streptococcus pyogenes Cas9; N representing any nucleotide) (Jinek et al., 2012). After the Cas9 RNP binds to an on-target DNA substrate, the target strand (TS) and non-target strand (NTS) of the DNA substrate are cleaved by HNH nuclease domain and RuvC nuclease domain, respectively (Gasiunas et al., 2012; Jinek et al., 2012; Sapranauskas et al., 2011). After catalysis in vitro, Cas9 remains stably bound to the cleaved DNA substrate (Singh et al., 2016; Sternberg et al., 2014). Engineering the active sites of the nuclease domains creates the Cas9_{dHNH} variant (Cas9 with H840A mutation) that cuts only the NTS, and the dCas9 variant (Cas9 with 1-1840A and D10A mutations) that is inactive for DNA cleavage (Jinek et al., 2012). CRISPR-mediated transcriptional activation and repression platforms (CRISPRa and CRISPRi) were developed utilizing dCas9 (Gilbert et al., 2013; Maeder et al., 2013; Mali et al., 2013a; Perez-Pinera et al., 2013; Qi et al., 2013).

DNA fluorescence in situ hybridization (DNA FISH) allows for direct visualization of specific DNA sequences in situ, making it a powerful tool to study chromatin conformation and gene localization (Beliveau et al., 2012; Boettiger et al., 2016; Levsky and Singer, 2003; Wang et al., 2016). Conventional DNA FISH requires harsh conditions such as high temperature and concentrated formamide to globally denature genomic DNA for probe hybridization, which risk disrupting the integrity of biological structures such as heat-labile epitopes of proteins and increase the likelihood of DNA FISH probes binding to off-target genomic DNA sequences that are exposed due to global denaturing. By exploiting the high binding affinity of dCas9 RNP to specific DNA sequences, fluorescently labeled dCas9 RNP has been adopted for genomic loci imaging in live cells or in fixed cells without global genomic DNA denaturation (Chen et al., 2013; Chen et al., 2016a; Deng et al., 2015; Hong et al., 2018; Ma et al., 2018; Neguembor et al., 2018; Qin et al., 2017; Wang et al., 2019). Visualization of non-repetitive genomic sequences has been achieved with dCas9-binding-based genomic imaging methods (Chen et al., 2013; Deng et al., 2015; Hong et al., 2018; Mao et al., 2019; Qin et al., 2017; Shao et al., 2018), but the signal-to-background ratio was compromised by non-specific binding of dCas9 RNP to off-targets in genomic DNA (Knight Spencer et al., 2017; Lakadamyali and Cosma, 2020; Wu et al., 2019). Cas9 RNP can tolerate up to eleven PAM-distal mismatches on the DNA substrate for stable binding in vitro (Singh et al., 2016; Sternberg et al., 2014). However, more than three PAM-distal mismatches drastically reduce or inhibit DNA cleavage activities of Cas9 RNP (Sternberg et al., 2015), indicating that the cleavage specificity of Cas9 RNP is much higher than its specificity for stable binding. Conformational activation of Cas9 is dependent of the base-pairing between guide RNA and target DNA, but it is independent of whether the nuclease domains are engineered to be catalytically dead or not (Anders et al., 2014; Dagdas et al., 2017; Huai et al., 2017; Jiang et al., 2016; Nishimasu et al., 2014; Sternberg et al., 2015). We therefore expect that Cas9 nickase variants such as Cas9_{dHNH} should have similar cleavage specificity as Cas9, and a genomic imaging method that relies on Cas9 or Cas9 nickase variants cleaving target DNA would have higher labeling specificity than Cas9-binding-based genomic imaging methods.

In this study, we show that the post-cleavage Cas9_{dHNH}-RNA-DNA ternary complex can recruit a 3′ to 5′ DNA helicase to unwind double-stranded DNA (dsDNA) beyond the protospacer. Exploiting this observation, we demonstrate a physiological-temperature DNA FISH method that leverages the high cleavage specificity of Cas9_{dHNH} to label target genomic DNA.

### Design

Previous cell-based studies suggested that the NTS in Cas9-RNA-DNA complex is exposed and available for annealing to an exogenous DNA strand (Richardson et al., 2016). Cleavage on the NTS reveals ˜17-nt of single-stranded DNA (ssDNA) with a 3′ hydroxyl end, the NTS 3′ flap (FIG. 1A) (Jinek et al., 2012). A recent single-molecule study showed that the NTS 3′ flap can be digested by a ssDNA-specific exonuclease, suggesting the 3′ hydroxyl end may be exposed to solvent (Wang et al., 2020). Rep-X is a highly processive 3′ to 5′ DNA helicase engineered from K col Rep helicase through conformational control (Arslan et al., 2015; Hua et al., 2018) based on mechanistic understanding of its activity regulation (Cheng et al., 2001; Comstock et al., 2015; Korolev et al., 1997; Lee et al., 2013). The ssDNA translocating and dsDNA unwinding activities of Rep-X are powered by ATP hydrolysis. We hypothesized that Rep-X can be loaded onto the NTS 3′ flap, translocate along the NTS, and unwind the dsDNA downstream of the protospacer (FIG. 1A). If the hypothesis is true, Cas9 RNP can function as a programmable loader of Rep-X to genomic DNA and the loaded Rep-X can unwind the downstream genomic DNA until it encounters an insurmountable blockade (FIG. 1B). If Rep-X loaded onto a cleaved NTS unwinds a long enough stretch of genomic DNA, the resulting ssDNA could be targeted by fluorescently labeled oligonucleotide probes for site specific imaging of genomic DNA in the cell (FIG. 1B). In this scheme that we call Genome Oligopaint via Local Denaturation Fluorescence in Situ Hybridization (GOLD FISH), after cell fixation, we first add the non-target strand nickase (Cas9_{dHNH} RNP) to bind and cleave specific DNA sequences in the cells. Next, we add Rep-X and ATP to unwind the dsDNA downstream of each Cas9_{dHNH} cleavage site and expose the FISH target sequences (FISH-TS, FIG. 1B). ATP is then removed. We surmised that the NTS that Rep-X translocates along may be removed from chromatin by Rep-X, for example if it hits the another nick generated by Cas9_{dHNH} nearby, or form secondary structures, or remain bound by Rep-X, preventing reannealing of the unwound genomic DNA (FIG. 1B). Finally, the Cy5-labeled FISH probes are added to hybridize with complementary FISH-TS sequences.

### Results

### Cas9_{dHNH} Exposes Cleaved NTS for Rep-X Superhelicase Loading

To test if Rep-X could be loaded onto the Cas9_{dHNH}-generated NTS 3′ flap and unwind the dsDNA beyond the protospacer at the single-molecule level, we developed a DNA helicase invasion assay (FIG. 1C). In this assay, the Cas9_{dHNH}-IRA-DNA ternary complex was assembled in Mg²⁺-containing imaging buffer and immobilized on a quartz slide through biotin-NeutrAvidin interaction (FIG. 1C). We internally labeled the PAM-distal region of the NTS with Cy5 so that if the loaded Rep-X fully unwound the 20-bp DNA downstream of the protospacer, fluorescence signal of Cy5 would be lost from the surface-tethered DNA (FIG. 1C). The internal Cy5-labeling on the NTS does not affect the DNA cleavage activity of Cas9 (Singh et al., 2018). When Rep-X and ATP were added together, the average number of fluorescent spots per image area decreased over time (FIGS. 1D and 1E), indicating that the Cy5-labeled DNA strand downstream of the NTS cleavage site was unwound by Rep-X and lost from the surface (FIG. 1C). Control experiments using dCas9 or without ATP did not show Cy5 spots decrease with time (FIG. 1F), indicating that both DNA nicking by Cas9_{dHNH} and ATP hydrolysis-driven DNA unwinding by Rep-X are necessary to remove the downstream NTS DNA. Together, our data suggest that Rep-X can invade into the Cas9_{dHNH}-RNA-DNA complex through the cleaved NTS and unwind the downstream dsDNA, supporting the design principle of GOLD FISH.

GOLD FISH Allows Efficient Labeling of a Repetitive Region within the MUC4 Gene at Physiological Temperature

We first tested GOLD FISH on a repetitive region within the MUC4 gene (MUC4-R) in IMR-90 cells, a human female diploid fibroblast strain (Nichols et al., 1977), so that a single guide RNA and a single Cy5-labeled FISH probe could be used to decorate the gene with multiple fluorophores. The GOLD FISH images of MUC4-R obtained using epifluorescence microscopy showed that 93% of cells contained 2 to 4 bright foci with low background (FIGS. 2A and 2B). Percentages of cells showing a particular number of foci are indicated above the corresponding histogram bin). We referred previously measured percentages of IMR-90 cells in G0/G1 (should have 2 foci) and S/G2/M (should have 4 foci) (Shi et al, 2007), and estimated the efficiency of detection to be 90%. Control experiments performed using dCas9 or without ATP showed weak or no detectable foci (FIGS. 6A and 6B), indicating that both DNA nicking by Cas9_{dHNH} and ATP hydrolysis-driven DNA unwinding by Rep-X are necessary to obtain bright GOLD FISH signals.

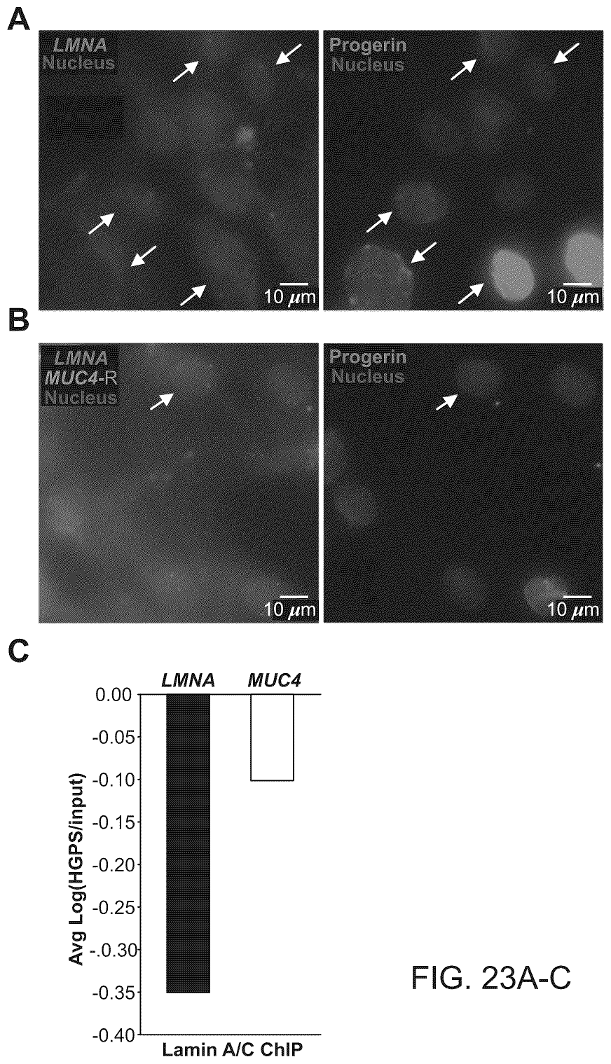

To test if GOLD FISH works in other cell types and if the FISH probes co-localize with Cas9 binding sites, we used an ATTO550-labeled guide RNA and performed GOLD FISH of MUC4-R in HEK293ft cells (co-localization assay, FIG. 2C). We found the guide RNA, and by inference Cas9_{dHNH} remained visibly bound to target DNA under our experimental condition, and 90% of Cy5 loci co-localized with ATTO550 loci (FIGS. 2D and 2E). Next, we access whether GOLD FISH reduces nuclear background arising from non-specific binding of Cas9_{dHNH} RNP. Of note, it is possible that some on- and off-target ATTO550-labeled Cas9_{dHNH} RNP dissociated from the cells during the Rep-X unwinding and FISH probe hybridization steps in GOLD FISH (FIG. 2E). To fairly compare GOLD FISH signals with signals from labeled Cas9_{dHNH} RNP binding, we also performed Cas9-mediated fluorescence in situ hybridization (CASFISH) against MUC4-R using either Cas9_{dHNH} or dCas9 with the ATTO550-labeled guide RNA (FIGS. 6C and 6D) (Deng et al., 2015). We measured the signal-to-background ratios of the ATTO550 foci and Cy5 foci from the co-localization assay (S/B_{ATTO550-colocalization} and S/B_{Cy5-colocalization}), as well as that of the CASFISH foci (S/B_{CASFISH-Cas9dHNH} and S/B_{CASFISH-dCas9}) (FIG. 2F). We found S/B_{Cy5-colocalization}; (17.4±6.6, mean±S.D.) was substantially greater than S/B_{ATTO550-colocalization} (2.7±1.4, mean±S.D.), S/B_{CASFISH-Cas9dHNH} (1.3-0.8, mean±S.D.) and S/B_{CASFISH-dCas9} (0.9±0.4, mean±S.D.), Control experiments performed without Cas9 enzyme showed that the nuclear background arising from cellular autofluorescence and non-specific intracellular binding of ATTO550-labeled guide RNA was negligible (FIG. 6E). Therefore, higher non-specific binding of labeled Cas9_{dHNH} RNP is responsible for the lower signal-to-background ratio of the ATTO550 foci in the co-localization assay and the CASFISH foci (FIG. 2F). S/B_{ATTO550-colocalization} being higher than S/B_{CASFISH-Cas9dHNH} also suggests that some non-specifically bound Cas9_{dHNH} RNP dissociated from the cells during the Rep-X unwinding and FISH probe hybridization steps in GOLD FISH (FIG. 2F). Together, these data indicate that GOLD FISH, which requires Cas9 cleavage of target DNA to achieve efficient labeling (FIGS. 23 and 6A), has much reduced non-specific labeling compared to the genomic imaging methods relying on labeled Cas9 RNP binding alone.

### GOLD FISH Enables Robust Labeling of Non-Repetitive DNA Sequences and Provides Chromatin Conformational Information

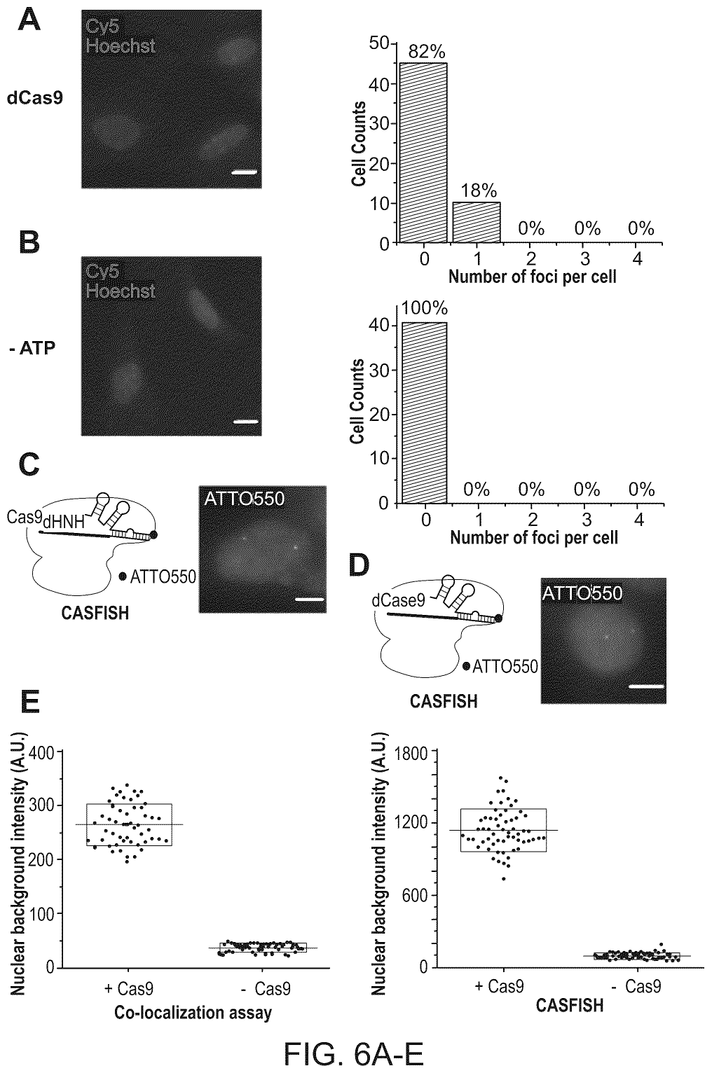

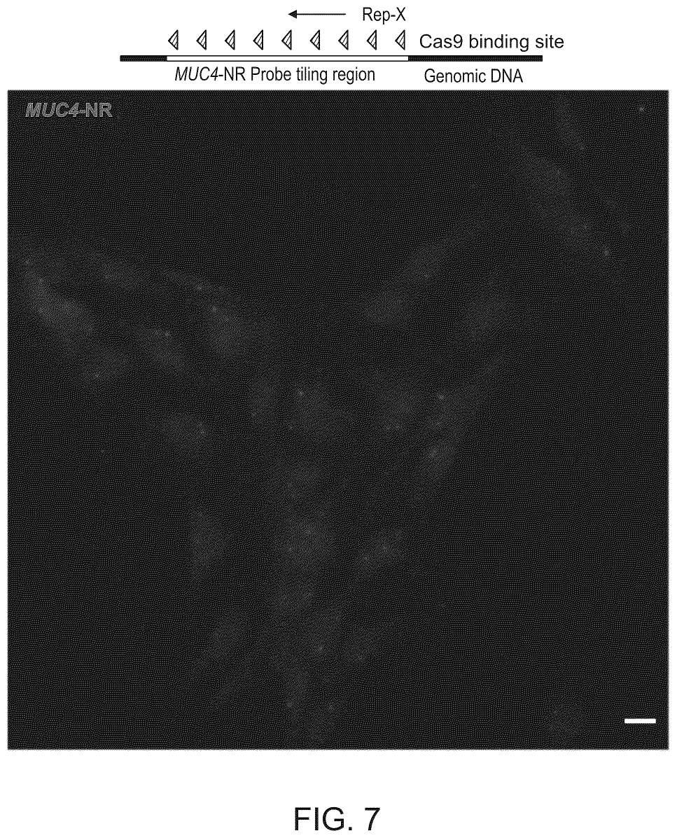

Low nonspecific binding of GOLD FISH should greatly facilitate non-repetitive loci imaging, which is generally much more challenging due to the need to include guide RNAs and FISH probes of multiple sequences at the same time. For example, if n different guide-RNA sequences and n different FISH probes are used, the total concentration of guide RNAs and FISH probes would have to be m and n times higher, respectively, to achieve the same signal level for each probe, potentially increasing background arising from nonspecific probe binding. A previous CASFISH study used 73 different guide RNAs to label a non-repetitive region within the MUC4 gene and observed compromised labeling efficiency and increased background (Deng et al., 2015). In order to test the capability of GOLD FISH in targeting non-repetitive DNA sequences, we designed 9 different guide RNAs (MUC4-NR guide-RNA set 1) targeting a 2.3-kilobases (kb) non-repetitive region within the MUC4 gene (MUC4-NR), with an approximate spacing of 300 base pair (bp) between them, and 57 different Cy5-labeled FISH probes that bind regions between the guide RNAs (FIG. 3A, top). Remarkably, GOLD FISH efficiently labeled the MUC4-NR region (FIG. 3A). 89% of cells had 2 to 4 FISH loci and the average signal-to-background ratio was 7.8 (FIGS. 3B and 7, percentages of cells with specific number of foci are shown above the histogram in FIG. 3B). The specificity of MUC4-NR FISH loci was verified by colocalization with MUC4-R loci (FIGS. 3A and 3C). The excellent labeling efficiency and signal-to-background ratio of MUC4-NR GOLD FISH confirm that it is capable of non-repetitive loci imaging without high nuclear background.

Rep-X can unwind thousands of base pairs of dsDNA in vitro (Arslan et al., 2015). To examine whether Rep-X is similarly processive on the genomic DNA, we performed GOLD FISH using the same fluorescently labeled probes targeting the MUC4-NR region but a new set of guide RNAs (MUC4-NR guide-RNA set 2). This set contains 11 different guide RNAs targeting a 2.4-kb region next to the MUC4-NR probe tiling region (FIG. 3D, top). Only 39% of cells had ≥2 detectable FISH loci, and the detectable loci had 30% lower signal-to-background ratio on average in comparison with using the MUC4-NR guide-RNA set 1 (FIGS. 3D and 3E). Next, we designed another set of guide RNAs (MUC4-I1) targeting a 2.5-kb repetitive region (˜50 copies) that is 30 kb away from the MUC4-NR probe tiling region (FIG. 3F, top). We found 91% cells did not have detectable focus (FIGS. 3F and 3G). Although it is possible that the MUC4-NR guide-RNA set 1, MUC4-NR guide-RNA set 2 and MUC4-I1 have different on-target activities which could lead to variability in labeling efficiency, the reduced labeling efficiency when unwinding initiates a few kb away (FIGS. 3D and 3F) is more likely because that Rep-X failed to efficiently unwind the chromatin from the Cas9 cleavage sites to the probe tiling region. We presume this is because the crowded nuclear environment and presence of nucleosomes inside cells prevent even a superhelicase from unwinding a very long stretch of chromatin. Therefore, we conclude that GOLD FISH locally denatures the targeted chromatin for FISH probe hybridization.

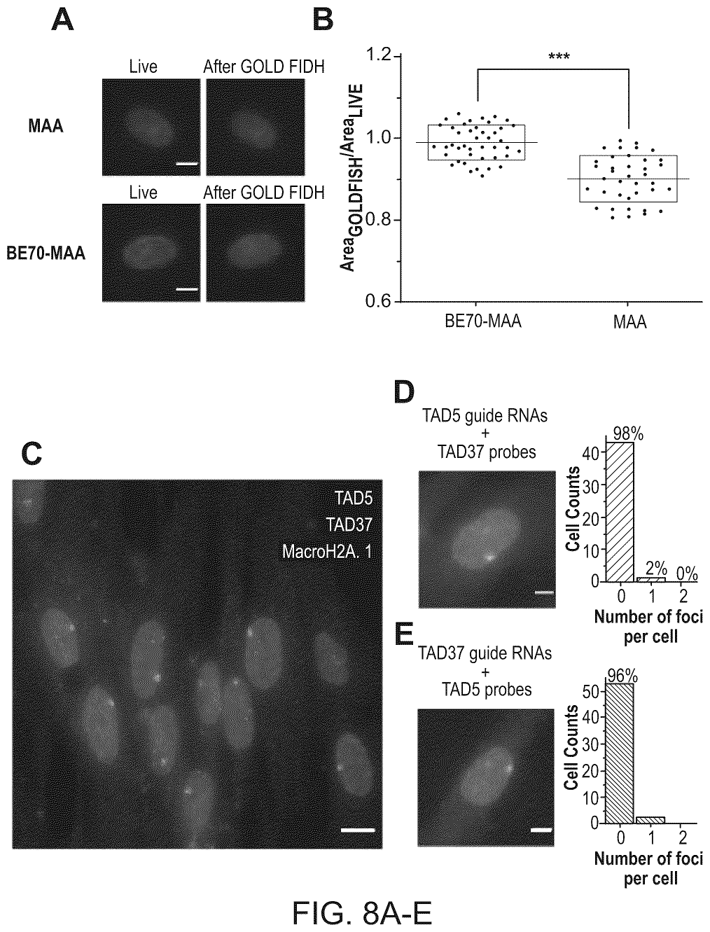

Next, we investigated if GOLD FISH can be used to quantitatively assess chromatin conformations. CASFISH and CRISPR/Cas9-mediated proximity ligation assay (CasPLA) are previously reported Cas9-mediated genomic imaging methods that are capable of labeling nonrepetitive loci in fixed cells (Deng et al., 2015; Zhang et al., 2018). The two methods use a solution of methanol and acetic acid (MAA) as the cell fixative. GOLD FISH experiments described above were also performed in MAA-fixed cells. However, it is known that fixation with methanol may cause a nuclear shrinkage (Boettiger et al., 2016). We found that, although the MAA fixation largely preserved the nuclear morphology, the projected nuclear area is reduced by 10% (FIGS. 8A and 8B). To overcome this issue, we combined a previously developed buffered ethanol (BE70) with MAA fixation (BE70-MAA) (Perry et al., 2016): the cells were fixed in BE70, and further permeabilized in MAA. We found that the BE70-MAA fixation method preserved the nucleus with a less than 1.2% reduction in projected nuclear area (FIGS. 8A and 8B). Next, we measured the spatial distance between two genomic regions by two-color GOLD FISH. Of the 40 topologically associated domains (TADs) previously identified in chromosome X (ChrX) of IMR-90 cells (Dixon et al., 2012), we chose two non-repetitive regions located in the 5^{th} and the 37^{th} TAD (TAD5 and TAD37, respectively). The genomic distance between TAD5 and TAD37 is 125 megabases (Mb, FIG. 4A, top). Two-color GOLD FISH against TAD5 and TAD37 was performed in the BE70-MAA-fixed IMR-90 cells (FIG. 4A). We were also able to distinguish inactive chromosome X (Xi) from active chromosome X (Xa) by concurrent MacroH2A.1 immunostaining (FIGS. 4A and 8C) (Costanzi and Pehrson, 1998). The average three-dimensional distance between TAD5 and TAD37 was 1.5 μm for Xi and 2.9 μm for Xa, close to the previously reported values (FIG. 4B) (Wang et al., 2016). Control GOLD FISH experiments using TAD5 guide RNAs with TAD37 probes or TAD37 guide RNAs with TAD5 probes showed that more than 96% cells had no detectable focus (FIGS. 8D and 8E), further supporting that GOLD FISH denatures genomic DNA locally. These results demonstrate that GOLD FISH can robustly target multiple non-repetitive DNA loci and provide chromatin structural information.

### Chromosomal Scale GOLD FISH

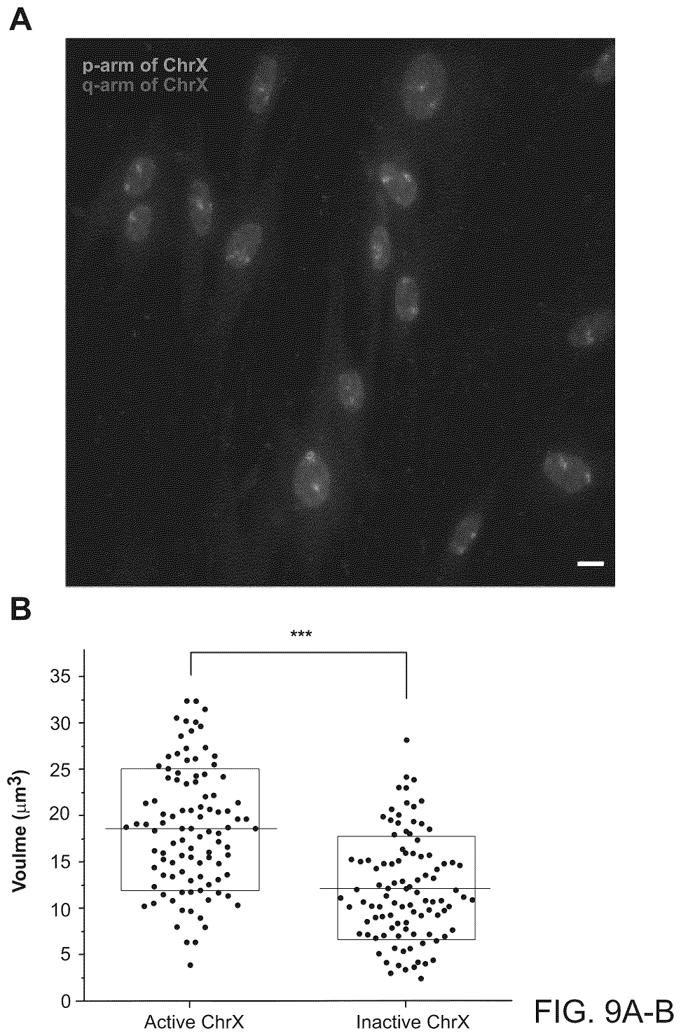

We further extended GOLD FISH to the chromosomal scale and imaged the p-arm and q-arm of ChrX in the BE70-MAA-fixed IMR-90 cells. We designed 3,287 guide RNAs and 2,307 FISH probes targeting all 40 TADs of ChrX in the IMR-90 cells. The FISH probes consist of unlabeled primary probes and fluorescently labeled readout probes (FIG. 4C). Each primary probe contains an encoding region complementary to genomic DNA, a readout region complementary to a specific readout probe, and two primer regions for amplification of the primary probe library (FIG. 4C). The probes against the p-arm and the q-arm of ChrX were labeled with Cy3 and Cy5, respectively (FIG. 4C). The GOLD FISH signals of the p-arm and the q-arm were cloud-like (FIGS. 4D and 9A), and MacroH12A.1 immunostaining was performed to distinguish Xi from Xa (FIG. 4D). The average center-of-mass distance between the p-arm and the q-arm for Xa is 28% larger than that for Xi (FIG. 4E), and the average volume of Xa is 53% greater than that of Xi (FIG. 9B). This is consistent with a previous finding that Xi adopts more compact conformations than Xa (Wang et al., 2016). Our data suggest GOLD FISH is scalable ranging from a single locus as short as 2.3 kb to chromosomal scale ‘paint’.

### GOLD FISH Allows for Rapid Identification of HER2 Gene Amplification in Tissue Samples

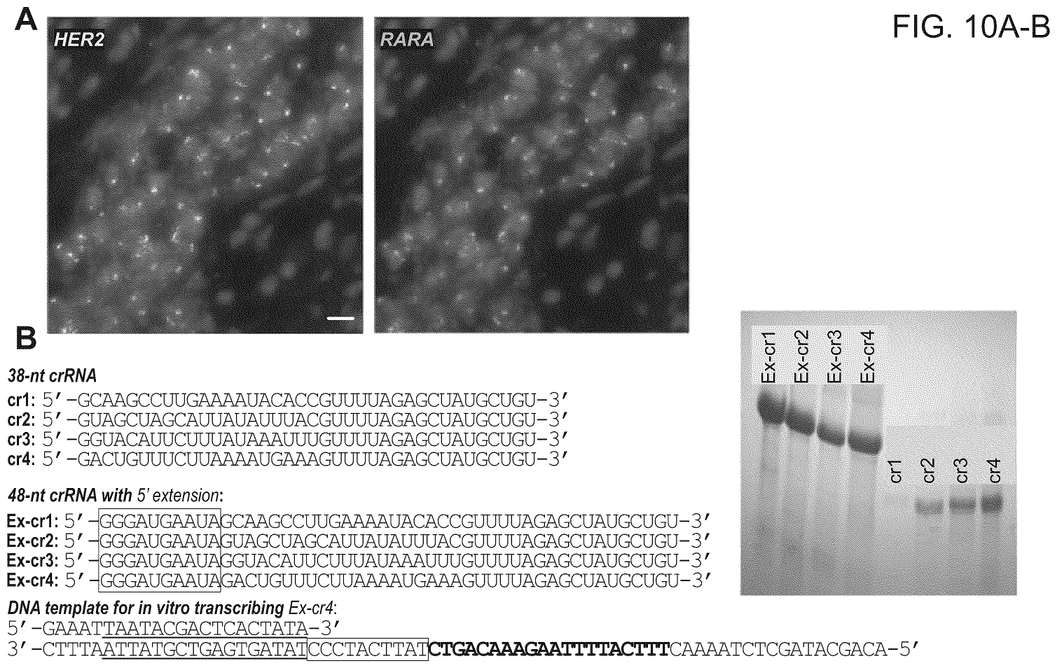

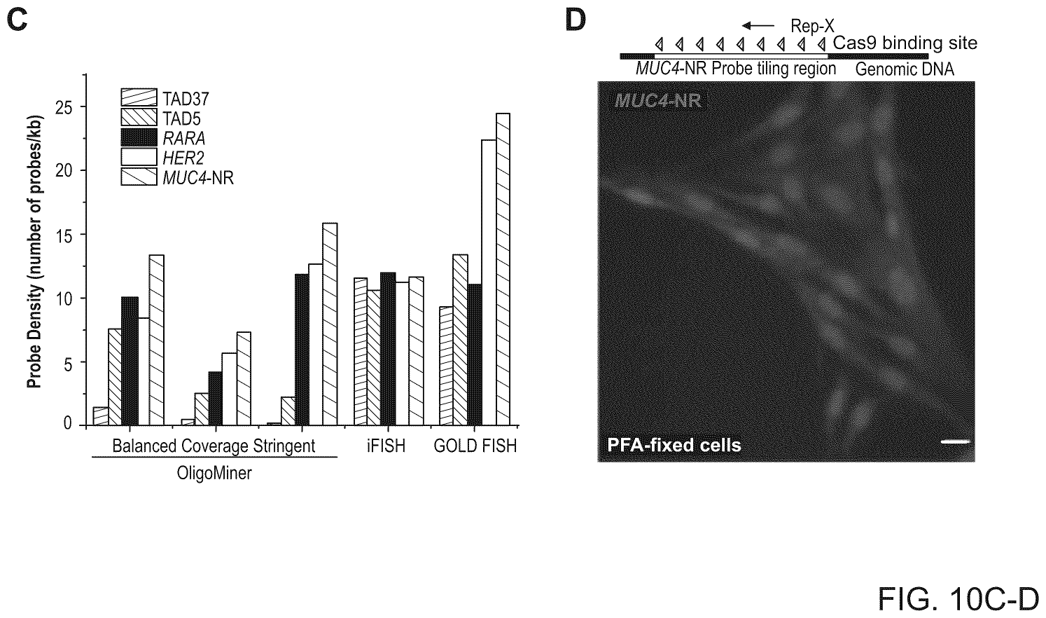

Finally, we show that GOLD FISH is applicable to non-repetitive DNA sequences in pathologic tissue samples. DNA FISH is widely used for diagnosis of molecular pathologies like Human Epidermal Growth Factor Receptor 2 (HER2) gene amplification in breast cancer patients, where the HER2 FISH spot number is compared to an enumeration gene or region of chromosome 17 (e.g. centromere region of chromosome 17 (CEP17)) to calculate the gene amplification state (FIG. 5A) (Furrer et al., 2015). Tissue samples fixed by non-crosslinking fixatives have several advantages compared to crosslinking-fixed tissue samples including higher quality and quantity of DNA, RNA and protein extraction (Oberauner-Wappis et al., 2016; Perry et al., 2016). Non-crosslinking fixation also allows faster probe hybridization to sequences of interest (Shaffer et al., 2013). However, the HER2 gene amplification testing in the non-crosslinking-fixed tissue samples requires an 18 to 24 hours crosslinking reaction prior to overnight conventional DNA FISH (Oberauner-Wappis et al., 2016), which extends the experimental procedures to days. To test whether GOLD FISH can rapidly detect non-repetitive sequences in the non-crosslinking-fixed tissue samples, we performed GOLD FISH targeting the HER2 gene and CEP17 in BE70-MAA-fixed human breast cancer tissue sections (10 μm thick), in parallel with immunostaining of HER2 protein. GOLD FISH efficiently labeled target sequences within 6 hours (including fixation time, FIG. 5B). By quantifying the numbers of HER2 and CEP17 foci per cell, we found 88% of cells had more than 4 copies of HER2 gene accompanied by high expression level of HER2 protein, while no more than 4 copies of CEP17 foci was observed (FIGS. 5B-5E, percentages of cells with specific number of foci are indicated above the histograms). The ratio of HER2/CEP17 foci numbers in each cell was 6.1±3.8 (mean±SD), indicating the HER2 gene amplification in the sample examined (FIG. 5F) (Wolff et al., 2013). Notably, although the Retinoic Acid Receptor Alpha (RARA) gene was suggested as a chromosome 17 enumeration gene (FIG. 5A) (Tse et al., 2011), we found RARA was co-amplified with HER2 gene (FIGS. 10A and 5F) (Varga et al., 2012). Therefore, RARA did not faithfully show the copy number of chromosome 17 in this tissue sample. Together, our analysis suggests that GOLD FISH can be directly applied to non-crosslinking fixed tissue samples for rapid DNA detection.

### Discussion

In this study, we repurposed the Cas9_{dHNH} RNP as a programmable loader of superhelicase to genomic DNA. We showed that Cas9_{dHNH} cleavage exposes a ssDNA region on the NTS, allowing Rep-X superhelicase to load on and unwind downstream dsDNA. Based on this, we developed GOLD FISH, a superhelicase-mediated physiological-temperature DNA FISH method. GOLD FISH leverages the high specificity of Cas9_{dHNH} cleavage to trigger targeted genomic DNA denaturing and shows several advantages when compared to other genomic imaging methods.

Current Cas9-mediated genomic imaging methods rely on the binding of directly or indirectly labeled dCas9 RNP to target DNA (Chen et al., 2013; Chen et al., 2016a; Deng et al., 2015; Hong et al., 2018; Ma et al., 2018; Mao et al., 2019; Qin et al., 2017; Shao et al., 2018; Wang et al., 2019). In contrast, GOLD FISH adds DNA cleavage as a prerequisite for efficient labeling (FIG. 6A). Cas9's cleavage specificity is much higher than its stable binding specificity (Singh et al., 2016; Sternberg et al., 2015; Sternberg et al., 2014). This could explain our observation that GOLD FISH shows excellent labeling specificity and avoids high nuclear background even when it targets non-repetitive loci.

Conventional DNA FISH denatures genomic DNA globally by heat and concentrated formamide treatments to enable probe hybridization. In contrast, GOLD FISH locally denatures targeted chromatin under much milder experimental conditions as we demonstrated through several examples. Targeted chromatin denaturing also reduces the likelihood of non-specific binding of FISH probes to the genome. CO-FISH and RASER FISH are DNA FISH methods that do not require heat denaturation, and RASER FISH has been used for super-resolution imaging of chromatin conformations (Brown et al., 2018; Williams and Bailey, 2009). However, CO-FISH and RASER FISH non-specifically and globally digest genomic DNA for probe hybridization, and require an overnight BrdU treatment in live cells prior to cell fixation (Brown et al., 2018; Williams and Bailey, 2009). BrdU may alter DNA stability, transcriptional/translational level, and lengthen the cell cycle (Taupin, 2007). In contrast, GOLD FISH does not require any treatment in live cells before cell fixation and therefore can also be applied to patient tissue samples as we demonstrated using human breast cancer tissue. The mild conditions also allow rapid GOLD FISH on tissue samples fixed by a non-crosslinking fixative. The HER2 GOLD FISH experiment in the 10-μm-thick non-crosslinking-fixed tissue sections took only 6 hours, while conventional HER2 DNA FISH in 2-μm-thick non-crosslinking-fixed tissue sections requires days (Oberauner-Wappis et al., 2016).

We have optimized the pipelines of guide-RNA and FISH probe synthesis to make GOLD FISH easy to implement and cost-effective. The oligonucleotide probes of GOLD FISH for targeting a few kilobases of non-repetitive genomic DNA were synthesized using an enzymatic approach (Gaspar et al., 2017). Oligonucleotides without any labeling or modification were purchased, and desired fluorophores were conjugated to the 3′ end of each oligonucleotide by using terminal deoxynucleotidyl transferase (TdT) (Gaspar et al., 2017). Each set of probes was labeled in a single TdT reaction. Ideally, a guide-RNA set for GOLD FISH targeting non-repetitive DNA sequences should have an equal amount of each guide-RNA species. However, the in vitro synthesis efficiencies of different canonical crRNAs can be dramatically different (FIG. 10B), likely because T7 transcription is sensitive to the first two or more nucleotides of the template DNA. This would cause different crRNA species to be present at different concentrations if transcribed together. In this case, different guide-RNA species must be synthesized individually, then combined in equal amounts, which is labor-intensive. To overcome this challenge, we adopted 5′ extended crRNA in most of guide-RNA designs in this study (Kocak et al., 2019). We found different crRNAs with a common 10-nt 5′ extension have similar synthesis efficiencies (FIG. 10B). This scheme enables the synthesis of multiple guide RNAs in a single reaction, giving similar synthesis efficiencies (therefore similar concentration) across the pool. Cas9 and Rep-X can be produced in large scales in terms of the amounts needed for GOLD FISH (Arslan et al., 2015; Jinek et al., 2012).

GOLD FISH has less stringent specificity requirements for designing FISH probes. Nonspecific annealing of probes to the rest of the genome is not a major concern because of targeted local denaturing of the genome. In contrast, conventional DNA FISH has stringent requirements to avoid annealing to the globally denatured genome. Therefore, GOLD FISH enables similar or higher probe density compared to the state-of-the-art. DNA FISH methods such as OligoMiner and iFISH (FIG. 10C) (Beliveau et al., 2018; Gelali et al., 2019). The higher probe density of GOLD FISH enabled efficient detection of a non-repetitive locus as short as 2.3 kb in human genome using epifluorescence microscopy (FIG. 3A). We have demonstrated GOLD FISH can ‘paint’ the X chromosome with differently colored fluorophores using the primary probes and readout probes (FIG. 4C), which is the scheme originally developed for multiplexed FISH experiments (Chen et al., 2015; Mateo et al., 2019; Wang et al., 2016). GOLD FISH differs from traditional DNA FISH only in the denaturation step, and therefore should be readily extendable to highly multiplexed FISH experiments.

It is intriguing how far Rep-X can unwind from the loading site in the complex and dense nuclear environment. The MUC4-NR data in FIG. 3 show that the distance between the guides and the FISH probes impacts the signal. When the distance between each FISH probe and the first ‘upstream’ guide for the probe is less than 400 bp, 89% cells showed 2-4 foci (FIGS. 3A and 3B). However, when the distance is on average 1.2 kb, only 39% cells showed 2-4 foci (FIGS. 3D and 3E). When the distance is ˜30 kb, 91% cells did not have detectable focus (FIGS. 3F and 3G). For other tested loci (MUC4-R, TAD5, TAD37, HER2 and RARA) which gave excellent signals, the distance between each FISH probe and the first ‘upstream’ guide of the probe was less than 400 bp. The data suggest that Rep-X should be able to efficiently unwind ˜400 bp, and larger distance between FISH probes and guides may lead to decreased labeling efficiency. Systematic investigation in the future may reveal the extent of unwinding more precisely and whether it depends on the chromatin environment.

GOLD FISH uses Cas9_{dHNH} RNP to create a 3′ flap for Rep-X loading. Therefore, the target locus should have enough sites that can be cleaved by Cas9 (e.g. nine Cas9 cleavage sites were sufficient for targeting MUC4-NR). The labeling efficiency of GOLD FISH may be compromised if crRNA has very low on-target activity (e.g., crRNA targeting a protospacer with very low or high GC content should be avoided) (Wang et al., 2014). The presence of nucleosomes and epigenetic modifications may also affect the ability of Cas9_{dHNH} to access and cleave target DNA, therefore influencing the labeling efficiency (Chen et al., 2016b; Horlbeck et al., 2016; Yarrington et al., 2018). crRNA designing tools with on-target activity prediction might be helpful (Cui et al, 2018). Because GOLD FISH uses oligonucleotide probes for hybridization with sequences of interest, targeting sequences that can form complexed structures such as G-quadruplex might lead to decreased labeling efficiency. Repeated sequences should not be problematic as potential target loci as long as there are PAM sequences for Cas9 targeting, as we have demonstrated for the MUC4-R repetitive locus (FIG. 2). The ‘difficult’ sequences mentioned above, and other repeated sequences may be tested in future studies to develop a robust guideline for GOLD FISH.

We unambiguously identified the HER2 amplification in the patient tissue sample using GOLD FISH (FIG. 5F), but it is possible that we underestimated the HER2 and RARA copy numbers in some cells. For example, two copies of HER2 loci are spatially within the diffraction limit resolution of epifluorescence microscopy cannot be distinguished. Combination of GOLD FISH with super-resolution microscopy would allow more accurate measurements of the gene copy numbers.

GOLD FISH does not require global heat denaturation of genomic DNA, which potentially improves the preservation of chromatin structures. However, crosslinking fixatives are not compatible with GOLD FISH. GOLD FISH of MUC4-NR did not show detectable signals in paraformaldehyde (PFA)-fixed cells (FIG. 10D), likely because the PFA crosslinking interfered with Cas9 finding its target DNA and/or because Rep-X cannot translocate/unwind long enough along the genomic DNA in PFA-fixed cells. Therefore, we used two non-crosslinking fixation methods in this work. The first method was MAA fixation (FIGS. 2 and 3). The second method was BE70-MAA fixation (FIGS. 4 and 5). We showed that the cells fixed using BE70-MAA had minimal reduction in projected nuclear area (FIGS. 8A and 8B), and the GOLD FISH-measured spatial distances between TAD5 and TAD37 were close to previously reported values measured using conventional DNA FISH (FIG. 4B) (Wang et al., 2016). But it is possible that some ultra-fine chromatin structures may be altered by BE70-MAA fixation. Further electron microscopy- or super-resolution microscopy-based study is required to assess the ultra-fine structure preservation of chromatin in GOLD FISH.

### Experimental Model and Subject Details

### Human Cell Lines

IMR-90 human female diploid fibroblast cells were purchased from American Type Culture Collection (ATCC, CCL-186) and cultured at 37° C. in 5% C02 in EMEM (ATCC, 30-2003) with 1 mM sodium pyruvate and 10% fetal bovine serum (FBS, ThermoFisher). IMR-90 cell line authentication was performed by the vendor. HEK293ft human female cells were a generous gift from the Regot lab (Johns Hopkins University School of Medicine). HEK293ft cell line authentication was not performed. HEK293ft cells were cultured at 37° C. in 5% C02 in DMEM (Corning) with 4.5 g/L glucose, L-glutamate, 1 mM sodium pyruvate, 1× antibiotic antimycotic solution (Sigma-Aldrich), and 10% FBS. Imaging dishes were coated with 1 μg/cm² fibronectin for 60 min, then washed with PBS before plating.

### Human Tissue Samples

Human breast cancer primary patient tissue was procured from ProteoGenex, which collected the samples with informed consent from the donor and approved by the Institutional Review Board/Independent Ethics Committee (IBR/IEC). The donor was 57 years old, female, with a breast cancer grade of G3. Samples were positive for estrogen receptor, progesterone receptor, and HER2 expression by immunofluorescence. We embedded the tissue in OCT media, froze it, sectioned it to 10 μm (OTF5000 cryostat—Bright Instruments), and adhered it collagen coated 21 mm² glass coverslips for imaging.

### Method Details

### Cas9 Expression and Purification

The expression plasmid of Cas9_{dHNH} (i.e. Cas9 (H840A)) was a gift from Jennifer Doudna (Addgene plasmid #39316; http://n2t.net/addgene:39316; RRID:Addgene 39316). To express and purify the Cas9, the plasmid was transformed into E. coii strain BL21 Rosetta 2 (DE3) (EMD Biosciences). The cells were grown in Terrific Broth (TB) at 37° C. to an optical density at 600 nm of 0.6. At this point IPTG was added to a final concentration of 0.5 mM to induce expression. Cells were left at 18° C. overnight (12-16 hrs) and harvested the next day. Cells were resuspended in lysis buffer (50 mM Tris, pH 7.5, 500 mM NaCl, 5% (v/v) glycerol and 1 mM TCEP) supplemented with protease inhibitor cocktail (Roche) and with Lysozyme (Sigma Aldrich). Cells were then lysed using an Emulsiflex-C5 homogenizer (Avestin). Insoluble material was pelleted at 18,000 rpm for 30 minutes at 4° C. and soluble lysate was collected and incubated with IMAC nickel affinity resin (Bio-Rad) for 30 minutes at 4° C. with gentle agitation. Resin was collected and washed in a column with 500 ml of lysis buffer. Resin was then incubated with lysis buffer supplemented with 250 mM Imidazole and first 10 ml of elute was collected. Removal of the 6His-MBP tag was performed by addition of 1 mg of TEV enzyme to the elute, incubated at 4° C. for 1 hour with no agitation. Sample was then introduced to IMAC resin again and the flow-through was collected and run over a HiLoad 26/600 S200 Superdex column (GE Healthcare) equilibrated with a buffer containing 100 mM potassium chloride, 20 mM tris pH 7.5 (at 25° C.), 5 mM magnesium chloride, and 5% (v/v) glycerol. Sample was then collected, concentrated with centrifugation columns, and then flash frozen in liquid nitrogen to be stored at −80° C. until further use. dCas9 was a generous gift from the laboratory of Jennifer Doudna (University of California, Berkeley).

### Rep-X Expression and Purification

Rep-X was prepared as previously described (Arslan et al., 2015). pET28a(+) vector containing rep (C18L/C43S/C167V/C612A/S400C) was transformed into E. coli B21(DE3) (Sigma-Aldrich, CMC00014) and plated out on LB agar containing 50 μg/ml kanamycin at 37° C. overnight. From the plate, a single colony was grown in 5 ml TB medium containing 50 μg/ml kanamycin at 30° C. overnight. The cells were transformed into 500 ml of TB medium containing 50 μg/ml kanamycin and grown at 37° C. When OD reached the range between 0.3 and 0.4, the cells were moved to an 18° C. incubator. When OD reaches 0.6 to 0.8, the cells were induced expression with 0.5 mM IPTG and continue growth overnight. The cells were harvested by centrifugation for 15 min at 5000 rpm and 4° C. The pellet was resuspended in 40 ml of the lysis buffer (50 mM Tris-HCl pH 7.5, 5 mM Imidazole, 200 mM NaCl, 20% (w/v) sucrose, 15% (v/v) glycerol, 17.5 ug/ml PMSF, and 0.2 mg/ml Lysozyme) and sonicate to lyse the cells. The lysed cell mix was centrifuged at 14,000 rpm at 4° C. for 30-60 min and collect the supernatant. The supernatant was stir-mixed with pre-equilibrated Ni-NTA resin for 1.5 hours at 4° C. Ni-NTA purification was performed by washing the protein-bound resin with buffer A (50 mM Tris-HCl pH 7.5, 5 mM Imidazole, 150 mM NaCl, 25% (v/v) glycerol), followed by buffer A1M (50 mM Tris-HCl pH 7.5, 5 mM Imidazole, 1 M NaCl, 25% (v/v) glycerol) to remove any DNA residue, and final washed the protein-bound resin with buffer A, then eluted the Rep variant with imidazole buffer (50 mM Tris-HCl pH 7.5, 205 mM Imidazole, 150 mM NaCl, 25% (v/v) glycerol). 20 μM eluted Rep variant was mixed with 100 μM BMOE crosslinker to self-crosslink into Rep-X. The reaction was stir-mixed at room temperature for 1 hour. The excess crosslinker and Imidazole was removed by an overnight dialysis and stored in Rep-X storage buffer (50% glycerol, 600 mM NaCl, 50 mM Tris-HCl pH 7.5) at −80° C.

### Preparation of Nucleic Acids for Single-Molecule Assays

DNA oligonucleotides were purchased from Integrated DNA Technologies (IDT). Cy5 N-hydroxysuccinimido (NHS) dyes were conjugated to DNA through a thymine modified with an amine group through a C6 linker (/iAmMC6T/). dsDNA targets were assembled by mixing the target strand (TS), non-target strand (NTS) and a 22-nt biotinylated adaptor strand at 1:1.25:1 ratio in T50 buffer (10 mM Tris-HCl pH 8, 50 mM NaCl) and incubating at 95° C. for 1 min, then cooling down to room temperature over 1 hour. The polyethylene glycol (PEG)-passivated flow chamber surface was purchased from Johns Hopkins University Slide Production Core for Microscopy. crRNA and tracrRNA were synthesized in vitro using HiScribe™ T7 Quick High Yield RNA Synthesis Kit (NEB, E2050S) according to the manufacturer's instructions. The guide RNA was annealed by mixing crRNA and tracrRNA at 1:1.25 ratio in Nuclease Free Duplex Buffer (IDT), and incubating at 95° C. for 30 seconds, then slowly cooling down to room temperature over 1 hour. The DNA and RNA sequences are listed in ‘Key Resources Table’.

### Microscopy and Data Acquisition for Single-Molecule Assays

Microscopy was performed on Nikon Eclipse Ti microscope and custom prism type TIRFM module. The system was driven by home-built software (smCamera 2.0). Nikon 60×/1.27 NA objective (CFI Plan Apo IR 60XC WI) was used. Illumination was provided by solid-state lasers (Coherent, 641 nm) combined and coupled to an optical fiber. Emission was collected using long-pass filters (T540LPXR UF3, T635LPXR UF3, T760LPXR UF3) and a custom laser-blocking notch filter (ZET488/543/638/750M) from Chroma. Images were recorded using an electron-multiplying charge-coupled device (EMCCD; Andor iXon 897).

### Single-Molecule Fluorescence Imaging and Quantification

For the DNA helicase invasion assay, Cy5-labeled dsDNA target (with 22-nt biotinylated adaptor strand) was immobilized on the PEG-passivated flow chamber surface using NeutrAvidin-biotin interaction. 100 nM Cas9 RNP was assembled by mixing 100 nM Cas9 and 100 nM wild-type gRNA and incubating for 10 min at room temperature in Mg²⁺-containing imaging buffer (20 mM Tris-HCl pH 8, 100 mM KCl, 5 mM MgCl₂, 5% (vol/vol) glycerol, 0.2 mg/ml BSA and saturated Trolox (>5 mM), 0.8% (w/v) dextrose) supplied with GLOXY (1 mg/ml glucose oxidase, 0.04 mg/ml catalase). The Cas9 RNP was flowed into the DNA-immobilized chamber and incubated for 20 min at room temperature. Short movies of 10 frames at 10 Hz with 641 nm laser excitation were taken at 20 different imaging views. The first 5 frame of each movie were averaged and Cy5 spot number per imaging view was measured as 0 min time point data. Then 100 nM Rep-X with 1 mM ATP in Mg²⁺-containing imaging buffer supplied with GLOXY were flowed into the chamber. The Cy5 spot number per imaging view was measured from 20 different imaging areas each again at different time points after flowing in Rep-X.

### Genome Sequences

The human genome assembly hg38 was used in this study and downloaded from genome.ucsc.edu. The coordinates of non-repetitive loci are listed below:
MUC4-NR (Chr3:195808789-195811123)
TAD5 (ChrX:18579431-18584379)
TAD37 (ChrX:143999562-144006499)
HER2 (Chr17:39706827-39710552)
RARA (Chr17:40348168-40355149)
   The coordinates of target sequences for p-arm/q-arm of ChrX ‘paint’ are listed in Table 2. (Table 2 follows claims section below.)

### Cas9 Binding Site and Probe Design for GOLD FISH

For GOLD FISH against a short target region (<10 kb), all potential Cas9 binding sites (i.e. all PAM sequences) within the target region were found using Benchling (benchling.com). Cas9 binding sites were chosen manually with the following constraints: adjacent Cas9 binding sites were generally spaced by 50 to 300 bp; all guide RNAs hybridized to the same strand (i.e. FISH-TS, FIG. 1B) so that Rep-X would translocating in the same direction along the other strand (i.e. Rep-X translocating strand, FIG. 1). The average spacing between consecutive Cas9 binding sites for MUC4-NR, TAD5, TAD37, HER2 and RARA are 266 bp, 166 bp, 163 bp, 93 bp and 188 bp, respectively. We arranged Cas9 binding sites relatively close to each other to increase the likelihood that Rep-X could peel off the Rep-X translocating strand between the two adjacent Cas9 binding sites (FIG. 1). Next, desired oligonucleotide probes against the FISH-TS were designed using Oligoarray 2.1 (FIG. 1B) (Rouillard, 2003). The DNA sequences between adjacent Cas9 binding sites were loaded into Oligoarray 2.1 with the following constraints: Length: 18- to 30-nt; Tm: 72° C. to 90° C.; % GC: 30-70; Max. Tm for structure: 54° C.; Min. Tm to consider X-hybrid: 54° C.; and there was no consecutive repeat of 5 or more identical nucleotides. For MUC4-R and MUC4-NR probes, no specificity filtering was performed. For TAD5, TAD37, HER2 and RARA, two specificity filters were applied: Probes with more than 30 non-specific bindings on human genome were removed; Probes that can non-specifically bind to human noncoding RNA and E. coli tRNA were also removed. We applied the probe filtering for the following reasons. First, if Cas9 and Rep-X non-specifically unwound a stretch of repetitive genomic DNA, and a probe that could non-specifically bind to the repetitive genomic DNA might give a detectable false positive signal. Second, RNA molecules in the cells might not be digested completely by RNAse. Probes annealing to abundant RNA (e.g. rRNA) or RNA molecules containing repetitive sequences might also give false positive signals. Third, E. coli tRNA was used as a blocking reagent. Forth, probe density remained high although the specificity filtering was applied (FIG. 0C). The excellent signal-to-background ratio with MUC4-NR GOLD FISH indicates the probe specificity filtering was not necessary in terms of keeping nuclear background low (FIGS. 3A and 3B). The colocalization of MUC4-R and MUC4-NR signals suggests false positive signal was rare even without the probe specificity filtering (FIG. 3C). The sequences of probes and template DNA for crRNA synthesis are listed in Table 1.

TABLE 1

| Template DNA for in vitro | |
|---|---|
| transcription of crRNAs | sequence (5′ −> 3′, template strand) |
| MUC4 repetitive region | caaaacagcatagctctaaaactcttcctgtcaccgacactctatagtgagtcgtattaatttc |
| (MUC4-R) | |
| MUC4-NR guide-RNA set 1 | acagcatagctctaaaactccagacatcgccgggctgctatagtgagtcgtattaatttc |
| (FIG. 3A) | acagcatagctctaaaacccttgtcgcttcccttgctctatagtgagtcgtattaatttc |
| | acagcatagctctaaaaccaaagactcagacacccagctatagtgagtcgtattaatttc |
| | acagcatagctctaaaaccttcaccagcatggcttctctatagtgagtcgtattaatttc |
| | acagcatagctctaaaacccacgtcctcacggtgggactatagtgagtcgtattaatttc |
| | acagcatagctctaaaacaccgttcaccactgcttttctatagtgagtcgtattaatttc |
| | acagcatagctctaaaacggtcagatgtgtgggtgccctatagtgagtcgtattaatttc |
| | acagcatagctctaaaactgtctagcccacctctgtcctatagtgagtcgtattaatttc |
| | acagcatagctctaaaacggagcacagtggggtttccctatagtgagtcgtattaatttc |
| MUC4-NR guide-RNA set 2 | acagcatagctctaaaacatgaagagcctaagactccctatagtgagtcgtattaatttc |
| (FIG. 3D) | acagcatagctctaaaacggttcaatcaccctccttcctatagtgagtcgtattaatttc |
| | acagcatagctctaaaactagatgaagggatatgaccctatagtgagtcgtattaatttc |
| | acagcatagctctaaaacttgcaaacccacaggaagcctatagtgagtcgtattaatttc |
| | acagcatagctctaaaactcggggtccgttcatgcagctatagtgagtcgtattaatttc |
| | acagcatagctctaaaacagggcgtgggggactcacctatagtgagtcgtattaatttc |
| | acagcatagctctaaaactagcaatgaagacacaaatctatagtgagtcgtattaatttc |
| | acagcatagctctaaaacgtgctgtgtcctagaacaactatagtgagtcgtattaatttc |
| | acagcatagctctaaaacagattttagtgccaagagactatagtgagtcgtattaatttc |
| | acagcatagctctaaaaccatgtgcagttgagaacatctatagtgagtcgtattaatttc |
| | acagcatagctctaaaacttcgaaatgcagctctcagctatagtgagtcgtattaatttc |
| TAD5 | acagcatagctctaaaacccctttatacattacagtgctatagtgagtcgtattaatttc |
| | acagcatagctctaaaaccaattttaatgctgattcactatagtgagtcgtattaatttc |
| | acagcatagctctaaaacaatttgggaaataatgactctatagtgagtcgtattaatttc |
| | acagcatagctctaaaactcgaggtgagtatgagattctatagtgagtcgtattaatttc |
| | acagcatagctctaaaactatctccttttctatcaccctatagtgagtcgtattaatttc |
| | acagcatagctctaaaacagtgatataagagaactcactatagtgagtcgtattaatttc |
| | acagcatagctctaaaacctattggcactcatgtctcctatagtgagtcgtattaatttc |
| | acagcatagctctaaaactgcataaaggtcataattactatagtgagtcgtattaatttc |
| | acagcatagctctaaaacgataatctttcttgcactgctatagtgagtcgtattaatttc |
| | acagcatagctctaaaacatacacaaatgcacaaggactatagtgagtcgtattaatttc |
| | acagcatagctctaaaactatcaagattgttggtagcctatagtgagtcgtattaatttc |
| | acagcatagctctaaaacaccacatcttcataacatcctatagtgagtcgtattaatttc |
| | acagcatagctctaaaacgtaaatataatgctagctactatagtgagtcgtattaatttc |
| | acagcatagctctaaaaccagttttctattcaaattactatagtgagtcgtattaatttc |
| | acagcatagctctaaaacggtacttacagaattaattctatagtgagtcgtattaatttc |
| | acagcatagctctaaaacatatgcccaatatctaatgctatagtgagtcgtattaatttc |
| | acagcatagctctaaaactctaaatgttcaatgaatgctatagtgagtcgtattaatttc |
| | acagcatagctctaaaacaacctgtatgctgaaaaatctatagtgagtcgtattaatttc |
| | acagcatagctctaaaacgtgtagcaaatggaagcatctatagtgagtcgtattaatttc |
| | acagcatagctctaaaacaaatttataaagaatgtacctatagtgagtcgtattaatttc |
| | acagcatagctctaaaacgtccgtgattgtgattactctatagtgagtcgtattaatttc |
| | acagcatagctctaaaactagcaaggacttcctaattctatagtgagtcgtattaatttc |
| | acagcatagctctaaaactttcattttaagaaacagtctatagtgagtcgtattaatttc |
| | acagcatagctctaaaacctaatattctcagtcattactatagtgagtcgtattaatttc |
| | acagcatagctctaaaactctacaatatgctagtttgctatagtgagtcgtattaatttc |
| | acagcatagctctaaaacggttttctttagcatagtgctatagtgagtcgtattaatttc |
| | acagcatagctctaaaaccatgaattattatttctttctatagtgagtcgtattaatttc |
| TAD37 | acagcatagctctaaaactcttgacctaatcacctccctattcatccctatagtgagtcgtattaa |
| | tttc |
| | acagcatagctctaaaactagtatctcatccttggccctattcatccctatagtgagtcgtattaa |
| | tttc |
| | acagcatagctctaaaacgcatctcatctaaatatccttattcatccctatagtgagtcgtattaat |
| | ttc |
| | acagcatagctctaaaacaactacaatggtgtggcaagtattcatccctatagtgagtcgtatt |
| | aatttc |
| | acagcatagctctaaaacacacccatcagggcaaacaatattcatccctatagtgagtcgtatt |
| | aatttc |
| | acagcatagctctaaaaccaaggttctaggtggccctatattcatccctatagtgagtcgtatta |
| | atttc |
| | acagcatagctctaaaacaatggctctactaagcattgtattcatccctatagtgagtcgtatta |
| | atttc |
| | acagcatagctctaaaactgcactctgtgagcctgcaatattcatccctatagtgagtcgtatta |
| | atttc |
| | acagcatagctctaaaaccagatggggcagccaagtagtattcatccctatagtgagtcgtat |
| | taatttc |
| | acagcatagctctaaaacttgagatcattcttccatgttattcatccctatagtgagtcgtattaat |
| | ttc |
| | acagcatagctctaaaactgtgattacaaattccaacttattcatccctatagtgagtcgtattaa |
| | tttc |
| | acagcatagctctaaaaccatcagaatatcctttaccatattcatccctatagtgagtcgtattaa |
| | tttc |
| | acagcatagctctaaaaccattgaaattgccctttatgtattcatccctatagtgagtcgtattaat |
| | ttc |
| | acagcatagctctaaaacttccatgtttataaggtatttattcatccctatagtgagtcgtattaatt |
| | tc |
| | acagcatagctctaaaacgcagattcaatgtctggtgatattcatccctatagtgagtcgtatta |
| | atttc |
| | acagcatagctctaaaacttcatgagggctttgccttctattcatccctatagtgagtcgtattaa |
| | tttc |
| | acagcatagctctaaaacgaacttatttatcttgtgtatattcatccctatagtgagtcgtattaatt |
| | tc |
| | acagcatagctctaaaactatcttggttattgtgaacatattcatccctatagtgagtcgtattaat |
| | ttc |
| | acagcatagctctaaaacccaggatggctaaatagtagtattcatccctatagtgagtcgtatt |
| | aatttc |
| | acagcatagctctaaaacgtatcacacaattaggtcattattcatccctatagtgagtcgtattaa |
| | tttc |
| | acagcatagctctaaaacttgtgaccagaaattgtatttattcatccctatagtgagtcgtattaat |
| | ttc |
| | acagcatagctctaaaaccaatgtttggtgtacatttatattcatccctatagtgagtcgtattaat |
| | ttc |
| | acagcatagctctaaaacgtagcatctgcatctgtttatattcatccctatagtgagtcgtattaat |
| | ttc |
| | acagcatagctctaaaaccactgtggtgggtggaatgatattcatccctatagtgagtcgtatt |
| | aatttc |
| | acagcatagctctaaaactggtagaaattacaggctgatattcatccctatagtgagtcgtatta |
| | atttc |
| | acagcatagctctaaaacttgatgctagaacgtctccatattcatccctatagtgagtcgtatta |
| | atttc |
| | acagcatagctctaaaactcctgctgatgtcattctcctattcatccctatagtgagtcgtattaat |
| | ttc |
| | acagcatagctctaaaacaagcacaaacattcgcaaagtattcatccctatagtgagtcgtatt |
| | aatttc |
| | acagcatagctctaaaacctttctgataatttatcgtctattcatccctatagtgagtcgtattaatt |
| | tc |
| | acagcatagctctaaaactagggagaaggtataacgattattcatccctatagtgagtcgtatt |
| | aatttc |
| | acagcatagctctaaaacgacttttctttgactacaattattcatccctatagtgagtcgtattaatt |
| | tc |
| | acagcatagctctaaaacggtactgtacttggcactggtattcatccctatagtgagtcgtatta |
| | atttc |
| | acagcatagctctaaaacaagtcacatctactctgacttattcatccctatagtgagtcgtattaa |
| | tttc |
| | acagcatagctctaaaacgagataaaattgcttaaagttattcatccctatagtgagtcgtatta |
| | atttc |
| | acagcatagctctaaaactctggaaaaatcatgctatttattcatccctatagtgagtcgtattaa |
| | tttc |
| | acagcatagctctaaaacaagcactggcattcttaatctattcatccctatagtgagtcgtattaa |
| | tttc |
| | acagcatagctctaaaacgatcaagtgaaagggttctttattcatccctatagtgagtcgtatta |
| | atttc |
| | acagcatagctctaaaacccataggtgcactgcttgtttattcatccctatagtgagtcgtattaa |
| | tttc |
| HER2 | acagcatagctctaaaacggtgggtctcgggactggcatattcatccctatagtgagtcgtatt |
| | aatttc |
| | acagcatagctctaaaaccattcatagggctcgtcagttattcatccctatagtgagtcgtatta |
| | atttc |
| | acagcatagctctaaaacgcgtctgcaatttgacaaactattcatccctatagtgagtcgtatta |
| | atttc |
| | acagcatagctctaaaacctctgtatctcagtttcttctattcatccctatagtgagtcgtattaatt |
| | tc |
| | acagcatagctctaaaaccagtacttggagatcttgggtattcatccctatagtgagtcgtatta |
| | atttc |
| | acagcatagctctaaaacctgcagtgggacctgcctcatattcatccctatagtgagtcgtatt |
| | aatttc |
| | acagcatagctctaaaacagagaaggtttcaatgacggtattcatccctatagtgagtcgtatt |
| | aatttc |
| | acagcatagctctaaaacgggccagcaaatagttacattattcatccctatagtgagtcgtatta |
| | atttc |
| | acagcatagctctaaaaccatcactcactagctgtgtatattcatccctatagtgagtcgtattaa |
| | tttc |
| | acagcatagctctaaaactctttgagtcccaccaccactattcatccctatagtgagtcgtatta |
| | atttc |
| | acagcatagctctaaaacctggatcaagacccctcctttattcatccctatagtgagtcgtatta |
| | atttc |
| | acagcatagctctaaaacgagagcggttggtgtctatctattcatccctatagtgagtgtatta |
| | atttc |
| | acagcatagctctaaaaccagagatgaagaggcacaggtattcatccctatagtgagtcgtat |
| | taatttc |
| | acagcatagctctaaaacgagacaggcagtgagagagatattcatccctatagtgagtcgtat |
| | taatttc |
| | acagcatagctctaaaacaagcctgttatcccaccccttattcatccctatagtgagtcgtatta |
| | atttc |
| | acagcatagctctaaaactgcagccggcagcacactgctattcatccctatagtgagtcgtatt |
| | aatttc |
| | acagcatagctctaaaacctgtggttgaagtggaggcatattcatccctatagtgagtcgtatt |
| | aatttc |
| | acagcatagctctaaaaccatatgctcccatttacagatattcatccctatagtgagtcgtattaa |
| | tttc |
| | acagcatagctctaaaacctccctaacacagtcagccatattcatccctatagtgagtcgtatta |
| | atttc |
| | acagcatagctctaaaaccgcatcagcacctgacatcctattcatccctatagtgagtcgtatt |
| | aatttc |
| | acagcatagctctaaaaccttggttgtgcagggggcagtattcatccctatagtgagtcgtatt |
| | aatttc |
| | acagcatagctctaaaacggggtagagagtagaaggcatattcatccctatagtgagtcgtat |
| | taatttc |
| | acagcatagctctaaaaccacctctcgcaagtgctccatattcatccctatagtgagtcgtatta |
| | atttc |
| | acagcatagctctaaaaccatctggatcctcaggactctattcatccctatagtgagtcgtatta |
| | atttc |
| | acagcatagctctaaaactggggcaaccaccgttctgatattcatccctatagtgagtcgtatt |
| | aatttc |
| | acagcatagctctaaaacctgcactccagcctgctgactattcatccctatagtgagtcgtatta |
| | atttc |
| | acagcatagctctaaaactggccccctctggaaaggggtattcatccctatagtgagtcgtatt |
| | aatttc |
| | acagcatagctctaaaacgctctgcagctattgaaagatattcatccctatagtgagtcgtatta |
| | atttc |
| | acagcatagctctaaaacggcgataaagcgagactctgtattcatccctatagtgagtcgtatt |
| | aatttc |
| | acagcatagctctaaaacccatgaatgccttcactcaatattcatccctatagtgagtcgtatta |
| | atttc |
| | acagcatagctctaaaacgtgatctcttccagagtctctattcatccctatagtgagtcgtattaa |
| | tttc |
| | acagcatagctctaaaaccctctttctcagtgctcacttattcatccctatagtgagtcgtattaat |
| | ttc |
| | acagcatagctctaaaacctatgggagaaaggtgggcatattcatccctatagtgagtcgtatt |
| | aatttc |
| RARA | acagcatagctctaaaaccaccactgctggcaggattctattcatccctatagtgagtcgtatta |
| | atttc |
| | acagcatagctctaaaacgacagacaaagcaaggcttgtattcatccctatagtgagtcgtatt |
| | aatttc |
| | acagcatagctctaaaactgcttgctgccaacttccactattcatccctatagtgagtcgtattaa |
| | tttc |
| | acagcatagctctaaaaccctgggtccccagccaagagtattcatccctatagtgagtcgtatt |
| | aatttc |
| | acagcatagctctaaaacggtatacctgcaccctggcatattcatccctatagtgagtcgtatta |
| | atttc |
| | acagcatagctctaaaacttgacctccgtcccacatcttattcatccctatagtgagtcgtattaa |
| | tttc |
| | acagcatagctctaaaacgggatctggaacccacaccctattcatccctatagtgagtcgtatt |
| | aatttc |
| | acagcatagctctaaaaccctcaggagaatcccagtactattcatccctatagtgagtcgtatt |
| | aatttc |
| | acagcatagctctaaaactgggtatggaggggagggagtattcatccctatagtgagtcgtat |
| | taatttc |
| | acagcatagctctaaaaccaactgaggcgggactcggctattcatccctatagtgagtcgtat |
| | taatttc |
| | acagcatagctctaaaacgagcacagacctgcccgtggtattcatccctatagtgagtcgtatt |
| | aatttc |
| | acagcatagctctaaaacctcagaattcaagcccctcctattcatccctatagtgagtcgtatta |
| | atttc |
| | acagcatagctctaaaactgtccagcctttccctcccttattcatccctatagtgagtcgtattaa |
| | tttc |
| | acagcatagctctaaaaccactgccctccccacaaaggtattcatccctatagtgagtcgtatt |
| | aatttc |
| | acagcatagctctaaaactaagcgaagggttccaggcttattcatccctatagtgagtcgtatt |
| | aatttc |
| | acagcatagctctaaaacgacacacaatcactcacaggtattcatccctatagtgagtcgtatt |
| | aatttc |
| | acagcatagctctaaaactgggactggctctgagcagctattcatccctatagtgagtcgtatt |
| | aatttc |
| | acagcatagctctaaaaccatgagcacagctgtggccctattcatccctatagtgagtcgtatt |
| | aatttc |
| | acagcatagctctaaaaccgtccacccccagcctgacatattcatccctatagtgagtcgtatt |
| | aatttc |
| | acagcatagctctaaaacggtgcgctttgcgcaccttctattcatccctatagtgagtcgtatta |
| | atttc |
| | acagcatagctctaaaacgggagctccttgggaagaggtattcatccctatagtgagtcgtatt |
| | aatttc |
| | acagcatagctctaaaaccagtcttaatgatgcacttgtattcatccctatagtgagtcgtattaa |
| | tttc |
| | acagcatagctctaaaacaggacacaggggcagtgccttattcatccctatagtgagtcgtat |
| | taatttc |
| | acagcatagctctaaaacgcacatctggctgctaggtgtattcatccctatagtgagtcgtatta |
| | atttc |
| | acagcatagctctaaaacaggtgaatctttttagtttttattcatccctatagtgagtcgtattaatt |
| | tc |
| | acagcatagctctaaaacaggttccccaccctggaggttattcatccctatagtgagtcgtatt |
| | aatttc |
| | acagcatagctctaaaacggatgaggtacacaacaagctattcatccctatagtgagtcgtatt |
| | aatttc |
| | acagcatagctctaaaacggtcatcctgcctacatgcatattcatccctatagtgagtcgtatta |
| | atttc |
| | acagcatagctctaaaaccaggatctgggtgcagaggatattcatccctatagtgagtcgtatt |
| | aatttc |
| | acagcatagctctaaaactctccgcatcatccatctcctattcatccctatagtgagtcgtattaa |
| | tttc |
| | acagcatagctctaaaactgctgtgtagttgcagaccatattcatccctatagtgagtcgtatta |
| | atttc |
| | acagcatagctctaaaaccccccactgtacagaaacggtattcatccctatagtgagtcgtatt |
| | aatttc |
| | acagcatagctctaaaaccaagcagggagagggttccctattcatccctatagtgagtcgtat |
| | taatttc |
| | acagcatagctctaaaacccccacacagggcagggcattattcatccctatagtgagtogtat |
| | taatttc |
| CEP17 | acagcatagctctaaaacttctttgatagttcaggttctatagtgagtcgtattaatttc |
| | acagcatagctctaaaactagtaaaggaaataacttcctatagtgagtcgtattaatttc |
| GOLD FISH probes | sequence (5′ −> 3′) |
| MUC4 repetitive region | gtggcgtgacctgtggatgctgagg |
| ((MUC4-R) | |
| MUC4 non-repetitive region | tcgtagccccggcattgg |
| (MUC4-NR) | |
| | tcctgccctgcctctcag |
| | aaccccgctccagtgctc |
| | gactcccagagcgaggct |
| | cgcagaggggcaagacct |
| | cagtcaccaagggcgggt |
| | ctggaggactggcagcca |
| | ggagctgccgcaatgagg |
| | ctggccgtcctggttcca |
| | ggctggagcaagtgcagg |
| | agcgcactccacggggaa |
| | agacccctctgcccctct |
| | ccctacacctccccaccct |
| | gatggtcgtggctgggaga |
| | cgagacaggtgagacgcaga |
| | ggccaagtctttgccgtgaa |
| | ggactgttggtgtgcaaggg |
| | cgtggagcgctgagtccttt |
| | aggctgggtcacactgaagc |
| | gctgggtgatggagacggaag |
| | agaagatcgggagacagcagc |
| | cccttccacctcgggatgttt |
| | agctcgggtttaaaagcctccat |
| | catgaactggcagatacgggacaa |
| | gagagaaaagagaacagagggcca |
| | gaaatttggtgtctactggtcgcc |
| | atgccaggcctgtgggagatgttcc |
| | gtccctctggcttcaggaccaagtt |
| | atttcttgggcgcatatttgaggag |
| | acctgcctatacttgagtctagggt |
| | caagggttaaaggataaggcacagc |
| | aggaacagagtcaaggagaaagatgt |
| | ccaagttcttgagtttccaaatctgtgatt |
| | agaggagaaaagtggggaagagg |
| | aagacagagggagaatgctcgaaa |
| | gcagagagaaagacaggcagagc |
| | tcatgtaaagctgcagggtgagag |
| | aggaccctgggaatggagaga |
| | tgggctcctccgtgtacagag |
| | cctcttccccacactttccagg |
| | gttcaagctggggttagagtctca |
| | agaacagtcgccgagggaaa |
| | ggagtgagtcagaaggcgagtgc |
| | aggctgagggcaaaggaaact |
| | tgaaggagtctccacgggaatc |
| | gcagcttggatttaggagcccc |
| | ttctggcccctgtgacca |
| | ccaggaagtggggtctgtgga |
| | cgtgacccggagaaggagtg |
| | cgacccgaagaagctaggcat |
| | tggagctgggccaggaga |
| | cgttccttttggctccctgaag |
| | ttgccggcaggaccctca |
| | tccttaaggaacagccctggc |
| | ccttggtctccagcactatcagtg |
| | accgaggacaccaaaactctcc |
| | ctttatgggcccctcctcggagaa |
| TAD5 | ttttcactctccctaaaggttttcaggcat |
| | taatgctaccccaaataataaccctgtata |
| | aagagcctttttaaacacagtgtgctg |
| | attgttttgctttctatctacaactttctc |
| | gcatctagaataattcatccaatacagagc |
| | cttccagcaattcgagcatattcta |
| | atgtagctttttactttctcaggtgg |
| | gcaccagtgaatagccttgattagct |
| | caaatatttctcatgtcttgcactttca |
| | acaaatgtaaagatctcaccacaatatcc |
| | tctacaattgggagtggatctataatagtt |
| | tccccaagtaagtttcaaagttcac |
| | aggctatagagatcaacattttgctct |
| | gtttcacattacctggtctacctgcag |
| | gaaccacctatataaagcctgattcag |
| | tgaagatacatactggagattgttggt |
| | caacgggcacttttcactaaggggtagag |
| | agtaaaggacaaaacagtttatggga |
| | tttcattgctataaaccacccacttc |
| | aatttgtgtcccactactgtaaaact |
| | atgacctttaaactattctcacaagtga |
| | tttctaggaataaaagacgatggcattc |
| | ccagttgacgtggagatctacc |
| | agactgttactagaatcattaccctagac |
| | ggctcctcattaatatccccaaactacag |
| | gcaaccaatgattcactatgcaca |
| | aaagggcagaggtagtgaaatctgtagatt |
| | cgcaattgtatttttagagtagaataccca |
| | aattatcaatgtctaatgctgaagcca |
| | acatgctttctgttacattcttctacataa |
| | aatataggccaacacatttcaaaaacc |
| | catgcagaccacaacttcaaaaat |
| | acactgttagatcactgtaatagcatcta |
| | ttctggttttatatctggagtgtcaagat |
| | tcctctaccaatttaagtcaaaacataaca |
| | caatttttcaacatgtgactcaaaagaatg |
| | gattaaacctggaacaactatcaacattc |
| | acaattttctactaggagagagtacaact |
| | taatacagagttatctacatgggagacac |
| | gacccatgaccctaacatacaatctaaaa |
| | ggattcttagttatatgtggaggtttacat |
| | gacaatgcctctatttcttgcttgtgtaat |
| | caaaatctttgaaaatactgagtgacaaga |
| | gccacaattaaaatctttcagatccagtat |
| | ttcctgaaaatcataatgctaacaaaatgc |
| | agaaatgccagaccaggagtactcttcct |
| | ttcctgaccatttacaacagtcttttctc |
| | gcgtactctgggagggga |
| | atgtttcttcataagacacgctttgaag |
| | caatcccagcgtaaaacaaaacccctaac |
| | gtacgtcagttttgagacatggaa |
| | ggttcactgtgaatagttatagagaatcag |
| | atcacccatctaactcaccaaacacaacat |
| | taccaattacagactctaccactactttta |
| | agcctttacataaacagtgatggaaacaa |
| | gtatttgcatatgtactgttgctgagcaa |
| | tcagctctoccaataaatgcaaacct |
| | tcccatgcttttataattattcagagca |
| | aaagtccccacaaaagctagaaat |
| | acaatgtattcactacaaaaccagtct |
| | agtagagtgttccatgtcaattgaaag |
| | tctcgcatgggctatttattaataatatcc |
| | gcgctagtaatactttaaacaacatcaaaa |
| | aagatagcaaagtccaaagttgtaact |
| | tgtaattagcattattgccttctgacag |
| | cattctattttgggacggtaactcaccc |
| TAD37 | tacgcttgccaaaaattcatatggt |
| | gcaattttattaggaggttggggat |
| | aaatgagataagactcctggggata |
| | caatttgcttcaagaccaagtgtac |
| | tacttgggaccagctctttctttct |
| | gggaatgggaatgtttgtcctg |
| | ggaaggtggggcattgtaaaaataa |
| | gccgtacctagcaaattcatgga |
| | agccacctctgagatcccagaac |
| | tccaacctgtgaaagcttcaaca |
| | aagagcttgcaagtccagcctgg |
| | aagatgccctagtgcccaggc |
| | actgcagaaatcccacacaag |
| | aagtggtaaagtttgatagcatcca |
| | cagaccaggtgcagcttaggccacc |
| | tctccctgtattccgttgcag |
| | tgccagggcttttgatgcaaaac |
| | cgccaggcgttattcatcaa |
| | ccaagtggacagttcatgagattag |
| | tgactcctaacagtgtaatctgaga |
| | aagaagaggaggtctgcagagatag |
| | aagagcacgctggacaaaagcc |
| | ggaacgggctttgaaattggataa |
| | agacagaaattcataccaggagtgg |
| | ctgtcatgttctacaggaagtcatc |
| | ccagtgcctacaactacaaatactt |
| | gctgatacctaattctgagtgcaat |
| | tatggatggcaaagcatataaagct |
| | actccagaactcgctgaagc |
| | agagcacatttgtcacatgccagag |
| | tgatatcaccaacgaagctcttct |
| | ttgccctttgtgcatacgattagct |
| | cattccagacccctgcaactgattg |
| | actttaagacactttcagtcagctt |
| | tggtctgaacaaacttaggacaaat |
| | cttatggaatgtctcacctgcagat |
| | acagtttaccaagagtcccactaat |
| | cctgacatacactgctagctgatat |
| | cagcatcttgtgtccacatctc |
| | ggagcagtcaggtatagagaaagg |
| | tgagagacaaatgagcacctgg |
| | gcaagatactgcccaggaaact |
| | catagctcagagccaagaaagag |
| | cctgtataacccctcctccaaaag |
| | agaaggcaagtaaaacagtttcact |
| | agtgtttagcccttaaacccctaac |
| | atgtgagcctgggagtcc |
| | ttcatgtgtctcctctgtctagtaa |
| | ttttctatctcttcaaccctgcatt |
| | agcacaaaaccccagagtagctg |
| | gaaccagccatatgaggagacactg |
| | tagacagcctatttagtgaggaagg |
| | cagaacacttgctctgtggtaggaa |
| | cttcttctgcccattccacaaatat |
| | acattgttcaatgaaatgcagcat |
| | tagagaattccctgcagtaaagtgc |
| | agacaaagcacacagagcatatttt |
| | ccatgaaggtaaacgctgactaaaa |
| | ttacacgcatgagaatacaaaccaa |
| | aatgaccttggaattatgcagaact |
| | caagcagttcaaagccaaagc |
| | cattcaccctcgtgctttcaggaat |
| | actttactttgcatgctcactacaa |
| | acacctgccccatggtta |
| HER2 | agtgagtttggatggggtgg |
| | tgctgttaaacctgcagtgtgc |
| | cagacatgaagctgcggc |
| | tgccagcatgatcatgtcccctg |
| | tgtttccgctaaatcttgtgctc |
| | tccacagaccctggaatgg |
| | gagtggaaaccaacctgctg |
| | ccaggcctttgctttcactg |
| | cgggaacaggactgctcagt |
| | ggtagggcgtgatctttatctcta |
| | tgctcatcgctcacaacca |
| | acaataccacccctgtcacag |
| | gtgctagacaatggagacccg |
| | ctctttgaggacaactatgccc |
| | tgagtgaagtgtacagtgaacaac |
| | tgggaagttgtagcttgcgtcagc |
| | gggaagacgccctcagaagattgg |
| | gcttgcagatataagggccaaaa |
| | cagtcacgctgcagtcctgtcagg |
| | gctttgttcttgttgcgggtg |
| | agcatggacaactcactcctc |
| | gagagctgcggtccacatgag |
| | ctcttaaccaccaagcagcatg |
| | gctcaccccacagagatctt |
| | cacaagaacaaccagctggc |
| | gccgcctacaccacccatttc |
| | ctcccaacttacaacccagtg |
| | cctcttctctcagacagcctg |
| | ccgtcctctcgctgttaga |
| | tgctgtgcagttggcctcgtgg |
| | ctctcagcctgtctgggtccct |
| | ctccctctgcttataggttgtgcc |
| | aatgcaggtgtcatacaggtga |
| | attgtcagagccgtgagtctca |
| | gttctccgatgtgtaagggctc |
| | cactgtctgtgccggtgg |
| | ggcgccactcagccctcat |
| | gtctccctagaaggtgatgctga |
| | tgaaagccagccacctgt |
| | ggcacaccagggcaaaac |
| | ctgcctggtactgccctat |
| | ctttgtgcccaatgtgctcta |
| | tctgactgcctggtatgtgcc |
| | tcattttggtggggaggtttg |
| | ccagctgtgtgactgcctgtc |
| | gtccatgcccaatcccgaggg |
| | tcacctacaacacagacacgt |
| | caggagtgggccttcagat |
| | ctgggctcagacctggggaac |
| | cacctatttactgatgggcgttta |
| | tctggttgttgtgaggggtaa |
| | tgggaatctcacagtgctgat |
| | ctcactagcacaatgaccttgaat |
| | gtgggatcctgcaccctc |
| | cctgatctccttagacaactacct |
| | tgcacgaagggccagggtatgtgg |
| | agttcctgtccctctgcgcatgc |
| | gtgcccgaggtacccactcactg |
| | gcagaggatggaacacagcggtg |
| | tcagtgtgctatggtctggg |
| | tgagacggccccttccccac |
| | tgttgacctgtcccggtatg |
| | gcccggaccctgatgctcat |
| | ggcacgatgacctgagacagtg |
| | cggagagctttgatgggtaaga |
| | agaagatctttgggagcctggc |
| | cagttaccagtgccaatatccag |
| | ctttatgtgaggctctgaaccg |
| | tgctctgtatagtgattggggtag |
| | ggaggcacacaaggacatttctgg |
| | cctttcacacttcctttacctcca |
| | tggttttcgtcgttttggtgggga |
| | acctagaccgtttatgcatctgta |
| | gaacatgggccagtgtctccctag |
| | acaaacagttccactttgtgtgtg |
| | gatgggaacagctgggct |
| | actaaggcctgggctttga |
| | tgctgggagtgatgtccaccc |
| | tacatctcagcatggccggacagc |
| | cactcctttaatctcaccctctgc |
| | ggaaggggtccgtggtaa |
| | ttccatgaaagtctgcagagtgtg |
| | gctttgggcctgagggagtactcc |
| RARA | gggagtcccagttttcttaagac |
| | tctgaagagatagtgcccagc |
| | agccattgagacccagagcag |
| | ggtctctaactgcccctcccc |
| | gagggacggtgaggcagggtgga |
| | cagcttgatgaggtcaatgggat |
| | gagttgaaggggtcattgggaag |
| | gtgagctggctgccgact |
| | tccagctggcagggcgta |
| | acagtttccccacaggtcct |
| | ccctgcctgaacctcaccatgg |
| | ttcagcctggagtagctatcc |
| | ccagcccgtgtatctgcctcctgg |
| | gtgtgtgttcgcctccatttctct |
| | catcctgcagtgttgaggc |
| | tgagaccgtagccaggcac |
| | gctcaggtaggcgatgggc |
| | ggagtgtgagtgccataggg |
| | gtttgcgagtctggctgg |
| | tggtgtgcgggctcacggttgag |
| | cagatgctcctaaagaccaaggg |
| | agtcccttctgattgtgagtctta |
| | caggccgtgcttggttttggggtg |
| | ggaagaaagaattgggacttctca |
| | ggatccaaaagccattgtctagtt |
| | ccatggagcctgagcctt |
| | cgcctccttgtctgcgtg |
| | ccggtgattgatgatgtcagagat |
| | tgtgttgaatcatgggtgttgcca |
| | gtgggtgacaactcaagacc |
| | actggagtgtcctgccacag |
| | ttgcacgaagagccccagac |
| | ggcacgggtgagatggttct |
| | cgtcagcagccaccacca |
| | gtgatctccaaattatgccagcta |
| | tcccctgcactttattgaatttgc |
| | ccgaatgataaacgtcttgtcaca |
| | agcagctgccatttcaatagaatt |
| | actccccacttgccccag |
| | agattctccccgccaggt |
| | gtggtgtgtgcggctcagcg |
| | cgaccccatcgcttctttaaa |
| | ctggctatggggtggggt |
| | gagctggagtagacgcgt |
| | agggctgggtgagtggaggc |
| | cccgtgaacgcgtgctgtgt |
| | gctggagtgcgtggcaatgc |
| | aacttgatgtgtgggttggg |
| | tacacgctgacgccggag |
| | tgcccaagcccgagtgct |
| | gcaagatctctgcgtccttcc |
| | gcctgcagggtgggatttgcc |
| | gcaaatacactacggtatggcttt |
| | acgtgtctctctggacattgacc |
| | gaccagatcaccctcctcaa |
| | cggcttcaccaccctcacca |
| | tgcccaccgcccaaatgtctgc |
| | tccttccagccagacagccaccct |
| | ataaagattcacgtaggagccagg |
| | cagccatccatccatttagccagt |
| | aagggctcggtccacctgtt |
| | gggaactgccaaagcctagg |
| | ggaggtttagattgtgctgcctgc |
| | tgaatgttgaggcgggtgatgggt |
| | ccagtgttccttagctcctagaaa |
| | ggttcgggttcagtccctgaa |
| | gtgctggtgccgagtgctcag |
| | tggtcctccgggagtgctggt |
| | ttcgccaaccagctgctg |
| | ggtatctctagagggcaggg |
| | cctggccgatgcatgaccctg |
| | gagggtcggaccaaccagggt |
| | tccagtgcttgttagttgctattt |
| | aggattacggtggtaacagatact |
| | ggtctctagctcatgaagttgatg |
| | tctgtaccctgcggcagc |
| CEP17 | taggcctcaaagaagtgcaattat/3cy3 sp/ |
| ssDNA used in hybridization | |
| buffer | sequence (5′ −> 3′) |
| Poly dT single-stranded DNA | ctagtttttttttttttttttt tttttttttttt |

For p-arm/q-arm of ChrX ‘paint’, Cas9 binding sites were found using custom-written scripts. The Cas9 binding sites were restricted within the central 300-kb regions of TADs in ChrX in IMR-90 cells (Dixon et al., 2012). All guide RNAs hybridized to the same strand (i.e. FISH-TS, FIG. 1B) so that Rep-X would translocating in the same direction along the other strand (i.e. Rep-X translocating strand, FIG. 1i). To increase the likelihood that Rep-X could peel off the Rep-X translocating strand (FIG. 1i), most of adjacent Cas9 binding sites were spaced by 50 to 200 bp. 50 to 100 Cas9 binding sites were designed for each TAD. Next, desired oligonucleotide probes against ChrX were designed (FIG. 4C). The probes were designed as previously described (Wang et al., 2016), with the following modifications: each primary probe contains 4 regions: a 20-nt forward priming region, a 20-nt readout region, a 20-nt encoding region for hybridization to genomic DNA and a 20-nt reverse priming region (FIG. 4C). To generate the encoding region sequences, the sequences between adjacent Cas9 binding sites (which spaced less than 200 bp) were loaded into Oligoarray 2.1 with the following constraints: Length: 20 nt; Tm: 72° C. to 90° C.; % GC: 30-70; Max. Tm for structure: 54° C.; Min. Tm to consider X-hybrid: 54° C.; and there was no consecutive repeat of 5 or more identical nucleotides; The generated encoding region sequences with more than 10 non-specific bindings on human genome or can bind to human non-coding RNA were removed. To generate the priming regions and readout region, computationally designed 25mer sequences (Xu et al., 2009) were loaded into Oligoarray 2.1 with the following constraints: Length: 20 nt; Tm: 75° C. to 90° C.; % GC: 40-60; Max. Tm for structure: 54° C.; Min. Tm to consider X-hybrid: 54° C.; and Sequence to avoid in the oligo: ‘GGGG;CCCC;TTTT;AAAA;ATATAT;TATATA;ACACAC;CACACA;CGCGCG;GCGCGC’; The generated priming region sequences and readout region sequences with at least 1 non-specific binding on human genome were removed. Amount thousands of candidate sequences satisfying all the constraints, four sequences were chosen as the priming regions and readout regions in this study (2 sequences for the priming regions and 2 sequences for the readout regions). Finally, the primary probes were assembled using the encoding region sequences, priming region sequences and readout region sequences as indicated in FIG. 4C. The primary probes with at least 9 non-specific bindings to human genome or at least one non-specific binding to E. coli tRNA or human non-coding RNA were excluded using BLAST+(here a ‘non-specific binding’ refers to the primary probe contains >16 nt homology sequence to an off-target sequence). The sequences of primers and template DNA for synthesizing the primary probes and the crRNAs are listed in Table 2. The sequences of readout probes are also listed in Table 2.

### Preparation of Guide RNAs for GOLD FISH

For GOLD FISH against a short target region (<10 kb), template DNA for in vitro transcribing crRNAs were purchased from IDT. The template DNA of a crRNA was partially double stranded, including a double-stranded T7 promoter region and a single-stranded template region (FIG. 10B). crRNAs were transcribed using HiScribe™ T7 Quick High Yield RNA Synthesis Kit (NEB). Different crRNAs have different protospacer sequences at 5′ end, and we found the transcription efficiency of crRNA heavily depends on its 5′ end sequence. Therefore, different crRNA would have different transcription efficiencies (FIG. 10B). To make the transcription efficiency homogeneous for different crRNA, we adopted 5′ extended crRNA (Kocak et al., 2019). A common 10-nt extension at 5′ of each crRNA made the transcription efficiency homogeneous (FIG. 10B). For MUC4-R, MUC4-NR, TAD5 and CEP17, canonical crRNAs were used, and each crRNA was transcribed separately. For TAD37, HER2 and RARA, the 5′ extended crRNAs were used, and each set of crRNAs were transcribed together in a single reaction. The transcribed crRNAs were purified by polyacrylamide gel electrophoresis.

For p-arm/q-arm of ChrX ‘paint’, an oligopool of template DNA (Twist Bioscience) was amplified to a dsDNA pool using Phusion® Hot Start Flex 2× Master Mix (NEB, M0536S) by limit-cycle PCR (no more than 10 cycles). The crRNAs for ChrX ‘paint’ was in vitro transcribed using the dsDNA pool and HiScribe™ T7 Quick High Yield RNA Synthesis Kit (NEB). The synthesized crRNA was purified using RNA Clean & Concentrator Kits (Zymo, R1017).

To assemble guide RNAs, the Alt-R® CRISPR-Cas9 tracrRNA (IDT) or Alt-R® CRISPR-Cas9 tracrRNA, ATTO™ 550 (IDT) and desired crRNAs were mixed at 1:1 ratio in Nuclease-Free Duplex Buffer (IDT) and incubated at 95° C. for 30 s, then slowly cooled down to room temperature over 1 hour.

### Synthesis of DNA Probe for GOLD FISH

For GOLD FISH against a short target region (<10 kb), designed DNA oligonucleotides (without any labeling/modification) were purchased from IDT, and fluorescently labeled as previously described (Gaspar et al., 2017). Briefly, to conjugate an amino-ddUTP at the 3′ end of each oligonucleotide, 66.7 μM DNA oligonucleotides, 200 μM Amino-11-ddUTP (Lumiprobe) and 0.4 U/μl Terminal Deoxynucleotidyl Transferase (TdT, Thermo Scientific, EP0162) were mixed in 1×TdT Reaction buffer (Thermo Scientific) and incubated overnight at 37° C. The reaction was cleaned up by ethanol precipitations and P4 beads (Bio-Rad, #1504124) spin columns. Next, the DNA oligonucleotides conjugated with amino-ddUTP were mixed with 100 μg of Cy3-NHS or Cy5-NHS (Lumiprobe or GE Healthcare) in 0.1 M sodium bicarbonate and incubated overnight at room temperature, and cleaned up by ethanol precipitations and P4 beads (Bio-Rad, #1504124) spin columns. We generally achieved ˜90% labeling efficiency. In some cases, unlabeled oligonucleotides were removed by high-performance liquid chromatography (HPLC).

For p-arm/q-arm of ChrX ‘paint’, an oligopool of template DNA for synthesizing primary probes were purchased from Twist Bioscience, and the primary probes were synthesized as previously described (Moffitt and Zhuang, 2016). In short, the oligopool of template DNA was amplified to a dsDNA pool using Phusion® Hot Start Flex 2× Master Mix (NEB) by limit-cycle PCR (no more than 10 cycles). One of the primers we used for the limit-cycle PCR contained a T7 promoter sequence. The dsDNA pool was cleaned up by using DNA Clean & Concentrator-100 (Zymo, D4029). Next, the dsDNA pool was further amplified and converted into single-stranded RNA (ssRNA) pool using HiScribe™ T7 Quick High Yield RNA Synthesis Kit (NEB). The ssRNA pool was converted back to desired primary probe pool by using Maxima H-reverse transcriptase (Thermo Scientific, EP0753) followed by alkaline hydrolysis. The primary probe pool was further purified to remove enzyme and excess dNTPs, and we found RNA Clean & Concentrator Kits (Zymo, R1017) worked excellently for this purpose. The primers for synthesizing the primary probes and Cy3- or Cy5-labeled secondary readout probes were purchase from IDT. The sequences of primers and template DNA for synthesizing the primary probes and the crRNAs are listed in Table 2. The sequences of readout probes are also listed in Table 2.

### Cost Estimate of GOLD FISH

Here we estimate the cost of GOLD FISH targeting a non-repetitive genomic locus (a few kb long). Assume GOLD FISH will be performed in an imaging dish with 12-millimeter-diameter glass bottom surface.

Guide RNAs. Alt-R® CRISPR-Cas9 tracrRNA can be purchased from IDT, each GOLD FISH experiment consumes ˜100 pmol of tracrRNA ($0.6 to $1.9). Template DNA for in vitro transcribing crRNAs can be purchased from IDT (oPools Oligo Pools). A set of template DNA strands (which can transcribe up to 47 different crRNAs) costs $99. The crRNAs can be in a single reaction using HiScribe™ T7 Quick High Yield RNA Synthesis Kit ($5.24 per reaction).

Oligonucleotide probes. DNA oligonucleotides without any labeling or modification can be purchase from IDT in a 500 picomole DNA Plate Oligo. The plate requires at least 96 oligonucleotides to be ordered. We found ˜60 probe oligos (on average 21-nt for each probe) would be enough for GOLD FISH to achieve excellent signals. Therefore, a plate containing 60 oligonucleotide probes and 36 random oligonucleotides (15-nt each) can be purchased from IDT ($180). To label the 60 oligonucleotide probes, terminal deoxynucleotidyl transferase (ThermoFisher, EP0162), amino-11-ddUTP (Lumiprobe) and NHS-form of fluorophores were used ($6 to $32).

Cas9 and Rep-X Each GOLD FISH experiment consumes ˜100 pmol of Cas9_{dHNH}. 100 pmol of Alt-R® S.p. Cas9 H840A Nickase V3 (IDT) costs $23 to $32. If Cas9_{dHNH} is produced in the lab, the cost on Cas9 per experiment can be substantially lower. We produced 200 nmol of Rep-X using reagents of less than $300, while each GOLD FISH experiment consumes only 45 pmol of Rep-X. Therefore, once Rep-X is produced in the lab, the cost on Rep-X per experiment can be very low.

### Fixation of Cultured Cell and Tissue Section

We adopted two different fixation methods for GOLD FISH: methanol-acetic acid (MAA) fixation and Buffered Ethanol (BE70)-based fixation (BE70-MAA). Both methods permeabilized cells during the fixation steps.

For GOLD FISH against MUC4-R and MUC4-NR, MAA fixation was used (except FIG. 10D). Cells were briefly washed once with PBS and fixed at −20° C. for 20 min in pre-chilled MAA solution (methanol and acetic acid mixed at 1:1 ratio), then washed three times (5 min each wash at room temperature unless indicated) with PBS.

For GOLD FISH against other genomic regions, BE70-MAA fixation was used. This fixation method has two steps: BE70 fixation and MAA treatment. The BE70 buffer were prepared as previously described (Perry et al., 2016). To make 50 ml of BE70 buffer, 2.5 ml of 10×PBS (pH 7.4) was mixed with 1 ml of 50% glycerol and 0.25 ml of glacial acetic acid. The mixture was adjusted to pH 4.3 by adding NaOH. The solution was then filled to 15 ml with distilled water and mixed with 35 ml of absolute (200 proof) EtOH. Cells were briefly washed once with PBS and fixed at room temperature for 25 min in BE70 buffer, then washed twice with PBS. Cells were then incubated at −20° C. for 20 min in pre-chilled MAA solution and washed three times with PBS. We found the incubation in MAA solution was necessary for efficient GOLD FISH labeling in the BE70-fixed cells, presumably because MAA solution further permeabilized the cells.

For the PFA-fixed cells (FIG. 10D), cells were fixed with 4% paraformaldehyde (PFA) in PBS at room temperature for 10 min. The cells were washed three times with PBS and incubated in freshly made 1 mg/ml sodium borohydride for 10 min at room temperature. The cells were washed twice with PBS, and further permeabilized with 0.5% (v/v) Triton X-100 in PBS for 10 min at room temperature. The cells were washed twice with PBS and incubated with 0.1 M HCl for 5 min at room temperature. Finally, the cells were washed three times with PBS.

For CASFISH, cells were fixed as previously described (Deng et al., 2015). Cells were fixed at −20° C. for 20 min in pre-chilled MAA solution, then washed three times with PBS.

### GOLD FISH

In this work, GOLD FISH was performed against different genomic sequences (e.g. repetitive, non-repetitive, and chromosome ‘paint’), and the GOLD FISH protocol has evolved with the development of the method. Therefore, individual GOLD FISH experiments were performed with different parameters (e.g. Cas9 RNP and oligo probe concentrations). To avoid confusions, here we describe a standard GOLD FISH protocol. Detailed protocols of each GOLD FISH experiment presented in this work can be found in Methods S1.

### Step 1: Targeted Chromatin Denaturation.

After the cell fixation, Cas9 RNP (20 nM to 40 nM per guide RNA species, e.g. the MUC4-NR guide-RNA set 1 contains 9 different guide RNAs, then the total concentration of guide RNA in this step would be 180 to 360 nM) was assembled by mixing equal amount of Cas9_{dHNH} and guide RNA in Binding-Blocking buffer (20 mM Hepes pH 7.5, 100 mM KCl, 5 mM MgCl₂, 5% (v/v) glycerol and 0.1% (v/v) TWEEN-20, 1% (w/v) BSA, freshly added 1 mM DTT, freshly added 0.1 mg/ml E. coli tRNA) and incubated for 10 min at room temperature. The cells were incubated in Binding-Blocking buffer for 10 min at 37° C., and the Cas9 RNP was added to the cells and incubated for 30 to 60 min at 37° C. After the incubation, free Cas9 RNP were removed. Rep-X (100 to 400 nM) in Binding-Blocking buffer supplied with 2 mM ATP were added to the cells and incubated at 37° C. for 30 min. The cells were washed three times (5 min each wash at room temperature) with PBS.

### Step 2: RNAse Digestion (Optional).

RNase Cocktail™ Enzyme Mix (Invitrogen, AM2286) was diluted 100 times in PBS and incubated with the cells for 1 hour at 37° C. The cells were washed three times (5 min each wash at room temperature) with PBS.

### Step 3: FISH Probe Hybridization.

The cells were incubated in freshly made hybridization buffer (10% to 20% (v/v) formamide, 2× saline-sodium citrate (SSC), 0.1 mg/ml E. coli tRNA, 10% (w/v) dextran sulfate, 2 mg/ml BSA) for 10 min at 37° C. Next, fluorescently labeled oligonucleotide probes (2.5 nM per probe, e.g. the MUC4-NR probe set contains 57 different oligonucleotide probes, then the total concentration of probes in this step should be 142.5 nM) in the hybridization buffer were applied to the cells and incubated for 1 hour at room temperature (repetitive targets) or 37° C. (non-repetitive targets). The cells were washed twice (15 min each wash) with wash buffer (20% formamide, 2×SSC) at 37° C. and once with PBS at room temperature for 5 min.

### Step 4: Preparation for Imaging.

(Optional) one drop of Hoechst 33342 Ready Flow™ Reagent (Invitrogen, R37165) was mixed with 2 ml of PBS and incubated with the cells for 2 min at room temperature. Finally, FISH-imaging buffer (20 mM Tris-HCl pH 7.5, 100 mM KCl, 5 mM MgCl₂, 5% (vol/vol) glycerol, 0.2 mg/ml BSA and saturated Trolox (>5 mM), 0.8% (w/v) dextrose) supplied with GLOXY (1 mg/ml glucose oxidase, 0.04 mg/ml catalase) was added to the cells for imaging.

### CASFISH

CASFISH experiments were performed as previously described (Deng et al., 2015). The fixed cells were incubated with CASFISH-blocking/reaction buffer (20 mM Hepes pH 7.5, 100 mM KCl, 5 mM MgCl₂, 5% (v/v) glycerol and 0.1% (v/v) TWEEN-20, 1% (w/v) BSA, freshly added 1 mM DTT) at 37° C. for 15 min. Five nM Cas9_{dHNH} or dCas9 was mixed with 5 nM ATTO550-labeled guide RNA and incubated in the CASFISH-blocking/reaction buffer for 10 min at room temperature, and stored on ice before next step. The assembled Cas9 RNP was applied to the cells and incubated at 37° C. for 30 min. After the incubation, the cells were washed three times with CASFISH-blocking/reaction buffer (5 min each at room temperature).

### Microscopy and Data Acquisition for GOLD FISH

Epifluorescence microscopy was performed on Nikon Eclipse Ti2 microscope with Nikon Plan Apo X 60× Oil objective and Intermediate Magnification switching of 1.0×/1.5×. The system was driven by NIS-Elements AR software. Illumination was provided by high power LED. Emission was collected using filter sets: ET—Sedat Quad (Chroma, 89100) for Hoechst 33342 channel, ET—Gold FISH (Chroma, 49304) for Cy3 or ATTO550 channel, ET—Cy5 Narrow Excitation (Chroma, 49009) for Cy5 channel, and ET—Cy7 (Chroma, 49007) for Alexa750 channel. Images were recorded as z-stacks (21 to 35 steps), with 200 nm or 300 nm step size using a digital CMOS camera (ORCA-Flash 4.0 C11440, Hamamatsu), except for several images which were recorded using an EMCCD camera (Andor iXon 888) (FIGS. 3F, 8D and 8E). TetraSpeck™ Microspheres (T7279, Invitrogen) were also imaged in the same way for correction of chromatic aberration between Cy3/ATTO550 channel and Cy5 channel.

Comparison of Live and after-GOLD FISH Cells

DNA in live IMR-90 cells was stained with Hoechst 33342 Ready Flow™ Reagent (Invitrogen, R37165) and imaged at the focus plane where the nuclear edges were the sharpest. The coordinates of imaged cells were recorded so that the same cells could be found again after GOLD FISH protocol. Next, some cells (FIG. 8A, top) were fixed using the BE70-based fixation method (i.e. BE70 fixation followed by MAA treatment). Other cells (FIG. 8A, bottom) were fixed using the MAA fixation method. Next, the protocol of GOLD FISH against TAD5 and TAD37 was performed on all cells. After the GOLD FISH, the previously imaged cells were found, and their nuclei were imaged again at the focus plane where the nucleus edges were sharpest. The images of nuclei before and after GOLD FISH were split into sub-images, and each sub-image contained only one nucleus. The area of nucleus in each sub-image were automatically measured using the ‘Threshold’ function with ‘IsoDATA’ parameter in Fiji/ImageJ (Schneider et al., 2012). For each cell, the ratio of nuclear area after GOLD FISH (Area_{GOLDFISH}) to nuclear area when the cell was alive (AreaLive) was calculated (FIG. 8B).

Data analysis for GOLD FISH

Generate representative images shown in FIGS. Images were processed using Fiji/ImageJ. Z-stack images were projected to a single plane using the ‘Max Intensity’ Z-Projection function. The contrasts of images were linearly adjusted by changing the minimum and maximum values using the ‘brightness/contrast’ function in Image J for optimal visualization purpose only. The correction of chromatic aberration between Cy3/ATTO550 channel and Cy5 channel was performed using the TetraSpeck™ Microspheres images with custom-written MATLAB scripts.

Foci fitting and signal-to-background measurement. FISH-quant was used to find foci in each cell and fitted with three-dimensional (3D) Gaussian function (Mueller et al., 2013). Spatial coordinates (x, y and z), amplitude (A_{signal}) and background (BGD_{FISH-quant}) were extracted from the 3D Gaussian fitting. The average background (BGD_{coverslip}) was calculated from multiple areas where there was no cell. To calculate signal-to-background ratio (S/B), we used

$$S / B = \frac{{A}_{signal}}{{BGD}_{FISH - quant} - {BGD}_{coverslip}}$$

TAD5 and TAD37 distance measurement. After the chromatic aberration correction, the distance between TAD5 and TAD37 was measured:

$$Distance = \sqrt{{( {x}_{TAD 5} - {x}_{TAD 37} )}^{2} + {( {y}_{TAD 5} - {y}_{TAD 37} )}^{2} + {( {z}_{TAD 5} - {z}_{TAD 37} )}^{2}}$$

Center of Mass distance and volume measurement. The Z-stack images of ChrX ‘paint’ were background-subtracted using the ‘Subtract background’ function in Fiji/ImageJ with rolling ball radius of 15 pixels. After the chromatic aberration correction between Cy3 channel and Cy5 channel, each ChrX was cropped into a small region manually. The mean and standard deviation of residual nuclear background (BGDₘₑₐₙ and BGD_{STDEV}) were measured. A threshold (T) was set at:

$$T = {BGD}_{mean} + 1.5 * {BGD}_{STDEV}$$

The pixels within the cropped region with intensities higher than T were selected. The center of mass coordinate ( [symbol] ) of p-arm or q-arm of ChrX was calculated using the coordinates [symbol] of each selected pixel and intensity I of each selected pixel as weighting factors:

$$\vec{CoM} = \frac{{Σ}_{{I}_{i} > T} ( {\vec{x}}_{i} * {I}_{i} )}{{Σ}_{{I}_{i} > T} {\vec{x}}_{i}}$$

The CoM distances between p-arm and q-arm of each ChrX were calculated:

$$Distance = \sqrt{{( \vec{{CoM}_{p - arm}} - \vec{{CoM}_{q - arm}} )}^{2}}$$

The volumes of each ChrX were calculated:

$$Volume = Number of selected pixels * pixel size$$

### Quantification and Statistical Analysis

FISH foci were fitted with three-dimensional Gaussian functions using FISH-quant to obtain foci number per cell, foci intensity and background (Mueller et al., 2013). The nuclear area of each cell was automatically measured using the ‘Threshold’ function with ‘IsoDATA’ parameter in Fiji/ImageJ (Schneider et al., 2012). Two or more cells with overlapping nuclei were excluded from quantifications. Statistical analyses were conducted using Student's t-test. n represents number of cells (except for FIG. 1E, where n represents number of imaging view measured). Standard deviation (SD) are shown in this work. OriginPro 2020 was used for the statistical analysis. Statistical details of experiments such as values of n can be found in the FIG. legends.

### Example 2: Methods—Detailed Protocols of GOLD FISH Experiments

GOLD FISH against MUC4 repetitive region (MUC4-R). 200 nM Cas9_{dHNH} (i.e. Cas9 (H840A)) and 200 nM guide RNA were mixed in Binding-Blocking buffer (20 mM HEPES pH 7.5, 100 mM KCl, 5 mM MgCl₂, 5% (v/v) glycerol and 0.1% (v/v) TWEEN-20, 1% (w/v) BSA, freshly added 1 mM DTT, freshly added 0.1 mg/ml E. coli tRNA) for 10 min at room temperature, then diluted to 20 nM Cas9 RNP in Binding-Blocking buffer before use. The cells were incubated in Binding-Blocking buffer for 10 min at 37° C., and the 20 nM Cas9 RNP was added to the cells and incubated for 30 min at 37° C. After the incubation, free Cas9 RNP were removed. 100 nM Rep-X in Binding-Blocking buffer supplied with 2 mM ATP were added to the cells and incubated at 37° C. for 30 min. The cells were washed three times (5 min each wash at room temperature) with PBS. And RNase Cocktail™ Enzyme Mix (Invitrogen, AM2286) were diluted 100 times in PBS and incubated with the cells for 1 hour at 37° C. The cells were washed three times (5 min each wash at room temperature) with PBS, and incubated in freshly made hybridization buffer (10% (v/v) formamide, 2× saline-sodium citrate (SSC), 0.1 mg/ml E. coli tRNA, 10% (w/v) dextran sulfate, 2 mg/ml BSA) for 10 min at room temperature. Next, 2.5 nM Cy5-labeled MUC4 repetitive region probe in the hybridization buffer were applied to the cells and incubated for 1 hour at room temperature in the dark. After hybridization, the cells were washed twice (15 min each wash) with wash buffer (20% formamide, 2×SSC) at 37° C., and once with PBS at room temperature for 5 min. One drop of Hoechst 33342 Ready Flow™ Reagent (Invitrogen, R37165) was mixed with 2 ml of PBS, and incubated with the cells for 2 min at room temperature. Finally, FISH-imaging buffer (20 mM Tris-HCl pH 7.5, 100 mM KCl, 5 mM MgCl₂, 5% (vol/vol) glycerol, 0.2 mg/ml BSA and saturated Trolox (>5 mM), 0.8% (w/v) dextrose) supplied with GLOXY was added to the cells for imaging.

GOLD FISH against MUC4-R using ATTO550 labeled guide RNA. The protocol of GOLD FISH against MUC4 repetitive region (MUC4-R) was used with the following modifications: ATTO550-labeled guide RNA was used; Five nM Cas9_{dHNH} RNP was added to the cells; The RNAse treatment step was omitted because the ATTO550 was labeled at the 5′ end of tracrRNA. RNAse might partially digest the tracrRNA and release ATTO550 from the tracrRNA; Cas9 wash buffer (20 mM Tris-HCl pH 8, 100 mM KCl, 5 mM MgCl₂, 5% (vol/vol) glycerol) was used instead of PBS; Hybridization buffer was supplied with 200 nM poly dT single-stranded DNA for further blocking non-specific single-stranded DNA binding sites in the cells; During the formamide wash steps, the cells were washed with (10% formamide, 2×SSC) at RT for 15 min, and washed again with (20% formamide, 2×SSC) at 37° C. for 10 min.

GOLD FISH against MUC4-R using ATTO550 labeled guide RNA in the absence of Cas9 (i.e. no Cas9 control, FIG. 6E). The protocol of GOLD FISH against MUC4 repetitive region (MUC4-R) using ATTO550 labeled guide RNA was used with the following modification: only 5 nM ATTO550-labeled guide RNA was added to the cells, Cas9_{dHNH} was omitted.

GOLD FISH against MUC4 non-repetitive region (MUC4-NR) using the MUC4-NR guide-RNA set 1. The commercial Alt-R® S.p. Cas9 H840A Nickase V3 (IDT) was used. 1.8 μM the Cas9 nickase variant and 1.8 μM guide RNAs were mixed in Binding-Blocking buffer for 10 min at room temperature, then diluted to 360 nM Cas9 RNP in Binding-Blocking buffer before use. The cells were incubated in Binding-Blocking buffer for 10 min at 37° C., and the 360 nM Cas9 RNP was added to the cells and incubated for 30 min at 37° C. After the incubation, free Cas9 RNP were removed. 300 nM Rep-X in Binding-Blocking buffer supplied with 2 mM ATP were added to the cells and incubated at 37° C. for 1 hour. The cells were washed three times (5 min each wash at room temperature) with PBS. And RNase Cocktail™ Enzyme Mix were diluted 100 times in PBS and incubated with the cells for 1 hour at 37° C. The cells were washed three times (5 min each wash at room temperature) with PBS, and incubated in freshly made hybridization buffer (20% (v/v) formamide, 2×SSC, 0.1 mg/ml E. coli tRNA, 10% (w/v) dextran sulfate, 2 mg/ml BSA, 200 nM poly dT single-stranded DNA) for 10 min at 37° C. Next, 142.5 nM Cy5-labeled MUC4 non-repetitive region probes in the hybridization buffer were applied to the cells and incubated for 1 hour at 37° C. in the dark. The cells were washed twice (15 min each wash) with wash buffer (20% formamide, 2×SSC) at 37° C., and once with PBS at room temperature for 5 min. One drop of Hoechst 33342 Ready Flow™ Reagent (Invitrogen) was mixed with 2 ml of PBS and incubated with the cells for 2 min at room temperature. Finally, the FISH-imaging buffer supplied with GLOXY was added to the cells for imaging.

GOLD FISH against MUC4-NR using the MUC4-NR guide-RNA set 2 (FIG. 3D-E). The protocol of GOLD FISH against MUC4 non-repetitive region (MUC4-NR) was used with the following modifications: 2.2 μM the Cas9 nickase variant and 2.2 μM guide RNAs were mixed in Binding-Blocking buffer for 10 min at room temperature, then diluted to 440 nM Cas9 RNP in Binding-Blocking buffer before use.

GOLD FISH against MUC4-NR using the MUC4-I1 guide RNA (FIG. 3F-G). The protocol of GOLD FISH against MUC4 non-repetitive region (MUC4-NR) was used with the following modifications: 250 nM the Cas9 nickase variant and 250 nM guide RNAs were mixed in Binding-Blocking buffer for 10 min at room temperature, then diluted to 40 nM Cas9 RNP in Binding-Blocking buffer before use.

GOLD FISH against MUC4-NR and MUC4-R (FIG. 3A). The protocol of GOLD FISH against MUC4 non-repetitive region (MUC4-NR) using the MUC4-NR guide-RNA set 1 was used with the following modifications: the 360 nM Cas9 RNP against MUC4-NR was supplied with 20 nM Cas9 RNP against MUC4-R; After the MUC4-NR probe hybridization and formamide wash steps, a 2^{nd} round of probe hybridization was performed: 2.5 nM Cy3-labeled MUC4 repetitive region probe in the hybridization buffer (10% (v/v) formamide, 2×SSC, 0.1 mg/ml E. coli tRNA, 10% (w/v) dextran sulfate, 2 mg/ml BSA) were applied to the cells and incubated for 1 hour at room temperature; the formamide wash steps were performed again after the 2^{nd} round hybridization.

GOLD FISH against MUC4-NR using the MUC4-NR guide-RNA set 1 in PFA-fixed cells (FIG. 10D). The protocol of GOLD FISH against MUC4 non-repetitive region (MUC4-NR) was used with the following modification: Cas9 RNP and Rep-X were added to the cells together in Binding-Blocking buffer supplied with 5 mM ATP and additional 5 mM MgCl₂, and incubated at 37° C. overnight. The higher ATP and MgCl₂ concentrations were used to support Rep-X's unwinding activity for a longer time.

GOLD FISH against TAD5 and TAD37. 2.3p M Cas9_{dHNH} nickase (H840A) and 1.15p M unlabeled guide RNAs against TAD5 were mixed in Binding-Blocking buffer supplied with another 5 mM MgCl₂ for 10 min at room temperature. 3.24 μM Cas9_{dHNH} nickase (H840A) and 1.62 μM unlabeled guide RNAs against TAD37 were mixed in Binding-Blocking buffer supplied with another 5 mM MgCl₂ for 10 min at room temperature. The cells were incubated in Binding-Blocking buffer for 10 min at 37° C. Next, 540 nM Cas9 RNP against TAD5 and 760 nM Cas9 RNP against TAD37 were mixed with 200 nM Rep-X and 4 mM ATP, and incubated with the cells at room temperature overnight. After the incubation, free Cas9 RNP were removed. The cells were again incubated with 200 nM Rep-X in Binding-Blocking buffer supplied with 2 mM ATP at 37° C. for 1 hour. The cells were washed three times (5 min each wash at room temperature) with PBS. And RNase Cocktail™ Enzyme Mix were diluted 100 times in PBS and incubated with the cells for 1 hour at 37° C. The cells were washed three times (5 min each wash at room temperature) with PBS, and incubated in freshly made hybridization buffer (20% (v/v) formamide, 2×SSC, 0.1 mg/ml E. coli tRNA, 10% (w/v) dextran sulfate, 2 mg/ml BSA) for 10 min at 37° C. Next, 166 nM Cy5-labeled TAD5 probes and 160 nM Cy3-labeled TAD37 probes in the hybridization buffer were applied to the cells and incubated for 1 hour at 37° C. in the dark. The cells were washed twice (15 min each wash) with wash buffer (20% formamide, 2×SSC) at 37° C., and once with PBS at room temperature for 5 min. For co-immunostaining of marcroH2A.1, the cells were incubated in IF-buffer (3% (w/v) BSA in PBS) for 20 min at room temperature. 250× diluted primary antibody (anti-marcroH2A.1, Abcam, ab183041) in IF-buffer was applied to the cells and incubated for 1 hour at room temperature, washed three times with PBS, incubated with 500× diluted secondary antibody (Goat anti-Rabbit Alexa Flour 750, Invitrogen, A21039) for 1 hour at room temperature, and washed three times with PBS. Finally, the FISH-imaging buffer supplied with GLOXY was added to cells for imaging.

GOLD FISH against p-arm and q-arm of ChrX. 2 μM Cas9_{dHNH} nickase (H840A) and 2 μM guide RNAs for ChrX ‘paint’ were mixed in Binding-Blocking buffer for 10 min at room temperature. The cells were incubated in Binding-Blocking buffer for 10 min at 37° C., and the 2 μM Cas9 RNP was added to the cells and incubated for 1 hour at 37° C. After the incubation, free Cas9 RNP were removed. 300 nM Rep-X in Binding-Blocking buffer supplied with 2 mM ATP were added to the cells and incubated at 37° C. for 1 hour. The cells were washed three times (5 min each wash at room temperature) with PBS. And RNase Cocktail™ Enzyme Mix were diluted 100 times in PBS and incubated with the cells for 1 hour at 37° C. The cells were washed three times (5 min each wash at room temperature) with PBS, and incubated in freshly made hybridization buffer (20% (v/v) formamide, 2× saline-sodium citrate (SSC), 0.1 mg/ml E. coli tRNA, 10% (w/v) dextran sulfate, 2 mg/ml BSA) for 10 min at 37° C. Next, 1.2 μM primary probes for ChrX ‘paint’ in the hybridization buffer were applied to the cells and incubated overnight at 37° C. in the dark. The cells were washed twice (15 min each wash) with wash buffer (20% formamide, 2×SSC) at 37° C., and incubated in freshly made hybridization buffer (10% (v/v) formamide, 2×SSC, 0.1 mg/ml E. coli tRNA, 10% (w/v) dextran sulfate, 2 mg/ml BSA) for 10 min at 37° C. Next, 10 nM Cy5-labeled readout probe for q-arm of ChrX and 10 nM Cy3-labeled readout probe for p-arm of ChrX in the hybridization buffer were applied to the cells and incubated for 1 hour at 37° C. in the dark. The cells were washed twice (15 min each wash) with wash buffer (30% formamide, 2×SSC) at 37° C., and once with PBS at room temperature for 5 min. The co-immunostaining of marcroH2A.1 was performed as described above. The FISH-imaging buffer supplied with GLOXY was added to cells for imaging.

GOLD FISH against HER2 and RARA on 10 μm tissue sections. 1.875 M Cas9_{dHNH} nickase (H840A) and 1.36 μM unlabeled guide RNAs against RARA were mixed in Binding-Blocking buffer supplied with additional 5 mM MgCl₂ for 10 min at room temperature. 1.875 μM Cas9_{dHNH} nickase (H840A) and 1.32 μM unlabeled guide RNAs against HER2 were mixed in Binding-Blocking buffer supplied with additional 5 mM MgCl₂ for 10 min at room temperature. The cells were incubated in Binding-Blocking buffer for 10 min at 37° C. 1.36 μM Cas9 RNP against RARA and 1.32 μM Cas9 RNP against HER2 were mixed at 1:1 ratio and incubated with the cells at 37° C. for 1 hour. Next, 1.36 μM Cas9 RNP against RARA and 1.32 μM Cas9 RNP against HER2 were mixed with 400 nM Rep-X and 4 mM ATP, and incubated with the cells at 37° C. for 1 hour. The cells were washed three times (5 min each wash at room temperature) with PBS. And RNase Cocktail™ Enzyme Mix were diluted 100 times in PBS and incubated with the cells for 1 hour at 37° C. The cells were washed three times (5 min each wash at room temperature) with PBS, and incubated in freshly made hybridization buffer (10% (v/v) formamide, 2×SSC, 0.1 mg/ml E. coli tRNA, 10% (w/v) dextran sulfate, 2 mg/ml BSA) for 10 min at 37° C. Next, 208 nM Cy5-labeled HER2 probes and 195 nM Cy3-labeled RARA probes in the hybridization buffer were applied to the cells and incubated overnight at 37° C. in the dark. The cells were washed twice (15 min each wash) with wash buffer (30% formamide, 2×SSC) at 37° C., and once with PBS at room temperature for 5 min. For co-immunostaining of HER2, the cells were incubated in IF-buffer for 20 min at room temperature. 200× diluted primary antibody (anti-HER2 erbb2, Cell Signaling Technology, 2165S) in IF-buffer was applied to the cells and incubated for 1 hour at room temperature, washed three times with PBS, incubated with 500× diluted secondary antibody (Goat anti-Rabbit Alexa Flour 750, Invitrogen, A21039) for 1 hour at room temperature, and washed three times with PBS. One drop of Hoechst 33342 Ready Flow™ Reagent was mixed with 2 ml of PBS, and incubated with the cells for 2 min at room temperature. Finally, the FISH-imaging buffer supplied with GLOXY was added to the cells for imaging.

GOLD FISH against HER2 and CEP17 on 10 μm tissue sections. The protocol of GOLD FISH against HER2 and RARA on 10 μm tissue sections was used with the following modifications: 40 nM Cas9 RNP against CEP17 instead of 1.36 μM Cas9 RNP against RARA was used; 2.5 nM Cy3-labeled CEP17 probe was used instead of 195 nM Cy3-labeled RARA probes.

### REFERENCES

Anders, C., Niewoehner, O., Duerst, A., and Jinek, M. (2014). Structural basis of PAM-dependent target DNA recognition by the Cas9 endonuclease. Nature 513, 569-573.
Arslan, S., Khafizov, R., Thomas, C. D., Chemla, Y. R., and Ha, T. (2015). Engineering of a superhelicase through conformational control. Science 348, 344.
Beliveau, B. J., Joyce, E. F., Apostolopoulos, N., Yilmaz, F., Fonseka, C. Y., McCole, R. B., Chang, Y., Li, J. B., Senaratne, T. N., Williams, B. R., et al. (2012). Versatile design and synthesis platform for visualizing genomes with Oligopaint FISH probes. Proceedings of the National Academy of Sciences of the United States of America 109, 21301-21306.
Beliveau, B. J., Kishi, J. Y., Nir, G., Sasaki, H. M., Saka, S. K., Nguyen, S. C., Wu, C. T., and Yin, P. (2018). OligoMiner provides a rapid, flexible environment for the design of genome-scale oligonucleotide in situ hybridization probes. Proceedings of the National Academy of Sciences of the United States of America 115, E2183-E2192.
Boettiger, A. N., Bintu, B., Moffitt, J. R., Wang, S., Beliveau, B. J., Fudenberg, G., Imakaev, M., Mirny, L. A., Wu, C. T., and Zhuang, X. (2016). Super-resolution imaging reveals distinct chromatin folding for different epigenetic states. Nature 529, 418-422.
Brown, J. M., Roberts, N. A., Graham, B., Waithe, D., Lagerholm, C., Telenius, J. M., De Ornellas, S., Oudelaar, A. M., Scott, C., Szczerbal, I., et al. (2018). A tissue-specific self-interacting chromatin domain forms independently of enhancer-promoter interactions. Nat Commun 9, 3849.
Chen, B., Gilbert, L. A., Cimini, B. A., Schnitzbauer, J., Zhang, W., Li, G. W., Park, J., Blackburn, E. H., Weissman, J. S., Qi, L. S., et al. (2013). Dynamic imaging of genomic loci in living human cells by an optimized CRISPR/Cas system. Cell 155, 1479-1491.
Chen, B., Hu, J., Almeida, R., Liu, H., Balakrishnan, S., Covill-Cooke, C., Lim, W. A., and Huang, B. (2016a). Expanding the CRISPR imaging toolset with Staphylococcus aureus Cas9 for simultaneous imaging of multiple genomic loci. Nucleic acids research 44, e75.
Chen, K. H., Boettiger, A. N., Moffitt, J. R., Wang, S., and Zhuang, X. (2015). Spatially resolved, highly multiplexed RNA profiling in single cells. Science 348, aaa6090.
Chen, X., Rinsma, M., Janssen, J. M., Liu, J., Maggio, I., and Goncalves, M. A. (2016b). Probing the impact of chromatin conformation on genome editing tools. Nucleic acids research 44, 6482-6492.
Cheng, W., Hsieh, J., Brendza, K. M., and Lohman, T. M. (2001). E. coli Rep oligomers are required to initiate DNA unwinding in vitro11 Edited by D. Draper. Journal of Molecular Biology 310, 327-350.
Comstock, M. J., Whitley, K. D., Jia, H., Sokoloski, J., Lohman, T. M., Ha, T., and Chemla, Y. R. (2015). Direct observation of structure-function relationship in a nucleic acid-processing enzyme. Science 348, 352.
Cong, L., Ran, F. A., Cox, D., Lin, S., Barretto, R., Habib, N., Hsu, P. D., Wu, X., Jiang, W., Marraffini, L. A., et al. (2013). Multiplex Genome Engineering Using CRISPR/Cas Systems. Science 339, 819.
Costanzi, C., and Pehrson, J. R. (1998). Histone macroH2A1 is concentrated in the inactive X chromosome of female mammals. Nature 393, 599-601.
Cui, Y., Xu, J., Cheng, M., Liao, X., and Peng, S. (2018). Review of CRISPR/Cas9 sgRNA Design Tools. Interdiscip Sci 10, 455-465.
Dagdas, Y. S., Chen, J. S., Sternberg, S. H., Doudna, J. A., and Yildiz, A. (2017). A conformational checkpoint between DNA binding and cleavage by CRISPR-Cas9. Science Advances 3, eaao0027.
Deng, W., Shi, X., Tjian, R., Lionnet, T., and Singer, R. H. (2015). CASFISH: CRISPR/Cas9-mediated in situ labeling of genomic loci in fixed cells. Proceedings of the National Academy of Sciences of the United States of America 112, 11870-11875.
Dixon, J. R., Selvaraj, S., Yue, F., Kim, A., Li, Y., Shen, Y., Hu, M., Liu, J. S., and Ren, B. (2012). Topological domains in mammalian genomes identified by analysis of chromatin interactions. Nature 485, 376-380.
Doudna, J. A., and Charpentier, E. (2014). The new frontier of genome engineering with CRISPR-Cas9. Science 346, 1258096.
Furrer, D., Sanschagrin, F., Jacob, S., and Diorio, C. (2015). Advantages and disadvantages of technologies for HER2 testing in breast cancer specimens. Am J Clin Pathol 144, 686-703.
Gasiunas, G., Barrangou, R., Horvath, P., and Siksnys, V. (2012). Cas9-crRNA ribonucleoprotein complex mediates specific DNA cleavage for adaptive immunity in bacteria. Proceedings of the National Academy of Sciences of the United States of America 109, E2579-2586.
Gaspar, I., Wippich, F., and Ephrussi, A. (2017). Enzymatic production of single molecule FISH and RNA capture probes. RNA.
Gelali, E., Girelli, G., Matsumoto, M., Wernersson, E., Custodio, J., Mota, A., Schweitzer, M., Ferenc, K., Li, X., Mirzazadeh, R., et al. (2019). iFISH is a publically available resource enabling versatile DNA FISH to study genome architecture. Nat Commun 10, 1636.
Gilbert, Luke A., Larson, Matthew H., Morsut, L., Liu, Z., Brar, Gloria A., Torres, Sandra E., Stern-Ginossar, N., Brandman, O., Whitehead, Evan H., Doudna, Jennifer A., et al. (2013). CRISPR-Mediated Modular RNA-Guided Regulation of Transcription in Eukaryotes. Cell 154, 442-451.
Hong, Y., Lu, G., Duan, J., Liu, W., and Zhang, Y. (2018). Comparison and optimization of CRISPR/dCas9/gRNA genome-labeling systems for live cell imaging. Genome Biol 19, 39.
Horlbeck, M. A., Witkowsky, L. B., Guglielmi, B., Replogle, J. M., Gilbert, L. A., Villalta, J. E., Torigoe, S. E., Tjian, R., and Weissman, J. S. (2016). Nucleosomes impede Cas9 access to DNA in vivo and in vitro. Elife 5.
Hua, B., Panja, S., Wang, Y., Woodson, S. A., and Ha, T. (2018). Mimicking Co-Transcriptional RNA Folding Using a Superhelicase. J Am Chem Soc 140, 10067-10070.
Huai, C., Li, G., Yao, R., Zhang, Y., Cao, M., Kong, L., Jia, C., Yuan, H., Chen, H., Lu, D., et al. (2017). Structural insights into DNA cleavage activation of CRISPR-Cas9 system. Nature Communications 8.
Jiang, F., Taylor, D. W., Chen, J. S., Kornfeld, J. E., Zhou, K., Thompson, A. J., Nogales, E., and Doudna, J. A. (2016). Structures of a CRISPR-Cas9 R-loop complex primed for DNA cleavage. Science 351, 867.
Jinek, M., Chylinski, K., Fonfara, I., Hauer, M., Doudna, J. A., and Charpentier, E. (2012). A Programmable Dual-RNA-Guided DNA Endonuclease in Adaptive Bacterial Immunity. Science 337, 816.
Knight Spencer, C., Tjian, R., and Doudna Jennifer, A. (2017). Genomes in Focus: Development and Applications of CRISPR-Cas9 Imaging Technologies. Angewandte Chemie International Edition 57, 4329-4337.
Kocak, D. D., Josephs, E. A., Bhandarkar, V., Adkar, S. S., Kwon, J. B., and Gersbach, C. A. (2019). Increasing the specificity of CRISPR systems with engineered RNA secondary structures. Nature biotechnology.
Korolev, S., Hsieh, J., Gauss, G. H., Lohman, T. M., and Waksman, G. (1997). Major Domain Swiveling Revealed by the Crystal Structures of Complexes of E. coli Rep Helicase Bound to Single-Stranded DNA and ADP. Cell 90, 635-647.
Lakadamyali, M., and Cosma, M. P. (2020). Visualizing the genome in high resolution challenges our textbook understanding. Nature methods.
Lee, K. S., Balci, H., Jia, H., Lohman, T. M., and Ha, T. (2013). Direct imaging of single UvrD helicase dynamics on long single-stranded DNA. Nat Commun 4, 1878.
Levsky, J. M., and Singer, R. H. (2003). Fluorescence in situ hybridization: past, present and future. Journal of cell science 116, 2833-2838.
Ma, H., Tu, L. C., Naseri, A., Chung, Y. C., Grunwald, D., Zhang, S., and Pederson, T. (2018). CRISPR-Sirius: RNA scaffolds for signal amplification in genome imaging. Nature methods 15, 928-931.
Maeder, M. L., Linder, S. J., Cascio, V. M., Fu, Y., Ho, Q. H., and Joung, J. K. (2013). CRISPR RNA-guided activation of endogenous human genes. Nature methods 10, 977-979.
Mali, P., Aach, J., Stranges, P. B., Esvelt, K. M., Moosburner, M., Kosuri, S., Yang, L., and Church, G. M. (2013a). CAS9 transcriptional activators for target specificity screening and paired nickases for cooperative genome engineering. Nature biotechnology 31, 833-838.
Mali, P., Yang, L., Esvelt, K. M., Aach, J., Guell, M., DiCarlo, J. E., Norville, J. E., and Church, G. M. (2013b). RNA-Guided Human Genome Engineering via Cas9. Science 339, 823.
Mao, S., Ying, Y., Wu, X., Krueger, C. J., and Chen, A. K. (2019). CRISPR/dual-FRET molecular beacon for sensitive live-cell imaging of non-repetitive genomic loci. Nucleic acids research 47, e131.
Mateo, L. J., Murphy, S. E., Hafner, A., Cinquini, I. S., Walker, C. A., and Boettiger, A. N. (2019). Visualizing DNA folding and RNA in embryos at single-cell resolution. Nature 568, 49-54.
Moffitt, J. R., and Zhuang, X. (2016). RNA Imaging with Multiplexed Error-Robust Fluorescence In Situ Hybridization (MERFISH). Methods Enzymol 572, 1-49.
Mueller, F., Senecal, A., Tantale, K., Marie-Nelly, H., Ly, N., Collin, O., Basyuk, E., Bertrand, E., Darzacq, X., and Zimmer, C. (2013). FISH-quant: automatic counting of transcripts in 3D FISH images. Nature methods 10, 277-278.
Neguembor, M. V., Sebastian-Perez, R., Aulicino, F., Gomez-Garcia, P. A., Cosma, M. P., and Lakadamyali, M. (2018). (Po)STAC (Polycistronic SunTAg modified CRISPR) enables live-cell and fixed-cell super-resolution imaging of multiple genes. Nucleic acids research 46, e30.
Nichols, W. W., Murphy, D. G., Cristofalo, V. J., Toji, L. H., Greene, A. E., and Dwight, S. A. (1977). Characterization of a new human diploid cell strain, IMR-90. Science 196, 60.
Nishimasu, H., Ran, F. A., Hsu, P. D., Konermann, S., Shehata, S. I., Dohmae, N., Ishitani, R., Zhang, F., and Nureki, O. (2014). Crystal structure of Cas9 in complex with guide RNA and target DNA. Cell 156, 935-949.
Oberauner-Wappis, L., Loibner, M., Viertler, C., Groelz, D., Wyrich, R., and Zatloukal, K. (2016). Protocol for HER2 FISH determination on PAXgene-fixed and paraffin-embedded tissue in breast cancer. Int J Exp Pathol 97, 202-206.
Perez-Pinera, P., Kocak, D. D., Vockley, C. M., Adler, A. F., Kabadi, A. M., Polstein, L. R., Thakore, P. I., Glass, K. A., Ousterout, D. G., Leong, K. W., et al. (2013). RNA-guided gene activation by CRISPR-Cas9-based transcription factors. Nature methods 10, 973-976.
Perry, C., Chung, J. Y., Ylaya, K., Choi, C. H., Simpson, A., Matsumoto, K. T., Smith, W. A., and Hewitt, S. M. (2016). A Buffered Alcohol-Based Fixative for Histomorphologic and Molecular Applications. J Histochem Cytochem 64, 425-440.
Qi, L. S., Larson, M. H., Gilbert, L. A., Doudna, J. A., Weissman, J. S., Arkin, A. P., and Lim, W. A. (2013). Repurposing CRISPR as an RNA-guided platform for sequence-specific control of gene expression. Cell 152, 1173-1183.
Qin, P., Parlak, M., Kuscu, C., Bandaria, J., Mir, M., Szlachta, K., Singh, R., Darzacq, X., Yildiz, A., and Adli, M. (2017). Live cell imaging of low- and non-repetitive chromosome loci using CRISPR-Cas9. Nat Commun 8, 14725.
Richardson, C. D., Ray, G. J., DeWitt, M. A., Curie, G. L., and Corn, J. E. (2016). Enhancing homology-directed genome editing by catalytically active and inactive CRISPR-Cas9 using asymmetric donor DNA. Nature biotechnology 34, 339-344.
Rouillard, J. M. (2003). OligoArray 2.0: design of oligonucleotide probes for DNA microarrays using a thermodynamic approach. Nucleic acids research 31, 3057-3062.
Sapranauskas, R., Gasiunas, G., Fremaux, C., Barrangou, R., Horvath, P., and Siksnys, V. (2011). The Streptococcus thermophilus CRISPR/Cas system provides immunity in Escherichia coli. Nucleic acids research 39, 9275-9282.
Schneider, C. A., Rasband, W. S., and Eliceiri, K. W. (2012). NIH Image to ImageJ: 25 years of image analysis. Nature methods 9, 671-675.
Shaffer, S. M., Wu, M. T., Levesque, M. J., and Raj, A. (2013). Turbo FISH: a method for rapid single molecule RNA FISH. PLoS One 8, e75120.
Shao, S., Chang, L., Sun, Y., Hou, Y., Fan, X., and Sun, Y. (2018). Multiplexed sgRNA Expression Allows Versatile Single Nonrepetitive DNA Labeling and Endogenous Gene Regulation. ACS Synth Biol 7, 176-186.
Shi, X., Seluanov, A., and Gorbunova, V. (2007). Cell divisions are required for L1 retrotransposition. Mol Cell Biol 27, 1264-1270.
Singh, D., Sternberg, S. H., Fei, J., Doudna, J. A., and Ha, T. (2016). Real-time observation of DNA recognition and rejection by the RNA-guided endonuclease Cas9. Nat Commun 7, 12778.
Singh, D., Wang, Y., Mallon, J., Yang, O., Fei, J., Poddar, A., Ceylan, D., Bailey, S., and Ha, T. (2018). Mechanisms of improved specificity of engineered Cas9s revealed by single-molecule FRET analysis. Nature structural & molecular biology 25, 347-354.
Sternberg, S. H., LaFrance, B., Kaplan, M., and Doudna, J. A. (2015). Conformational control of DNA target cleavage by CRISPR-Cas9. Nature 527, 110-113.
Sternberg, S. H., Redding, S., Jinek, M., Greene, E. C., and Doudna, J. A. (2014). DNA interrogation by the CRISPR RNA-guided endonuclease Cas9. Nature 507, 62-67.
Taupin, P. (2007). BrdU immunohistochemistry for studying adult neurogenesis: paradigms, pitfalls, limitations, and validation. Brain Res Rev 53, 198-214.
Tse, C. H., Hwang, H. C., Goldstein, L. C., Kandalaft, P. L., Wiley, J. C., Kussick, S. J., and Gown, A. M. (2011). Determining true HER2 gene status in breast cancers with polysomy by using alternative chromosome 17 reference genes: implications for anti-HER2 targeted therapy. J Clin Oncol 29, 4168-4174.
Varga, Z., Tubbs, R. R., Wang, Z., Sun, Y., Noske, A., Kradolfer, D., Bosshard, G., Jochum, W., Moch, H., and Ohlschlegel, C. (2012). Co-amplification of the HER2 gene and chromosome 17 centromere: a potential diagnostic pitfall in HER2 testing in breast cancer. Breast Cancer Res Treat 132, 925-935.
Wang, H., Nakamura, M., Abbott, T. R., Zhao, D., Luo, K., Yu, C., Nguyen, C. M., Lo, A., Daley, T. P., La Russa, M., et al. (2019). CRISPR-mediated live imaging of genome editing and transcription. Science 365, 1301.
Wang, S., Su, J.-H., Beliveau, B. J., Bintu, B., Moffitt, J. R., Wu, C.-t., and Zhuang, X. (2016). Spatial organization of chromatin domains and compartments in single chromosomes. Science 353, 598.
Wang, T., Wei, J. J., Sabatini, D. M., and Lander, E. S. (2014). Genetic Screens in Human Cells Using the CRISPR-Cas9 System. Science 343, 80.
Wang, Y., Mallon, J., Wang, H., Singh, D., Hyun Jo, M., Hua, B., Bailey, S., and Ha, T. (2020). Real-time observation of Cas9 postcatalytic domain motions. Proceedings of the National Academy of Sciences 118.
Williams, E. S., and Bailey, S. M. (2009). Chromosome orientation fluorescence in situ hybridization (CO-FISH). Cold Spring Harbor protocols 2009, pdb prot5269.
Wolff, A. C., Hammond, M. E., Hicks, D. G., Dowsett, M., McShane, L. M., Allison, K. H., Allred, D. C., Bartlett, J. M., Bilous, M., Fitzgibbons, P., et al. (2013). Recommendations for human epidermal growth factor receptor 2 testing in breast cancer: American Society of Clinical Oncology/College of American Pathologists clinical practice guideline update. J Clin Oncol 31, 3997-4013.
Wu, X., Mao, S., Ying, Y., Krueger, C. J., and Chen, A. K. (2019). Progress and Challenges for Live-cell Imaging of Genomic Loci Using CRISPR-based Platforms. Genomics Proteomics Bioinformatics 17, 119-128.
Xu, Q., Schlabach, M. R., Hannon, G. J., and Elledge, S. J. (2009). Design of 240,000 orthogonal 25mer DNA barcode probes. Proceedings of the National Academy of Sciences 106, 2289.
Yarrington, R. M., Verma, S., Schwartz, S., Trautman, J. K., and Carroll, D. (2018). Nucleosomes inhibit target cleavage by CRISPR-Cas9 in vivo. Proceedings of the National Academy of Sciences of the United States of America 115, 9351-9358.
Zhang, K., Deng, R., Teng, X., Li, Y., Sun, Y., Ren, X., and Li, J. (2018). Direct Visualization of Single-Nucleotide Variation in mtDNA Using a CRISPR/Cas9-Mediated Proximity Ligation Assay. J Am Chem Soc 140, 11293-11301.

### Example 3: Achieving Single Nucleotide Sensitivity in Direct Hybridization Genome Imaging

Single-nucleotide variation (SNV) is the most common type of mutation and is associated with many diseases¹. Although sequencing approaches can detect SNVs, they do not report on spatial information. Fluorescence in situ hybridization (FISH) can reveal the three-dimensional location of genomic sites of interest through annealing of fluorescently labeled oligonucleotide probes to denatured chromosomal DNA, but it generally cannot differentiate highly similar sequences. Advanced FISH-based methods have been developed to detect SNVs by targeting RNA molecules²⁻⁸, which requires the target RNA to be actively transcribing, thereby excluding nongenic regions and inactive or stochastically expressed genes, or by visualizing SNVs in DNA⁹⁻¹². Endogenous nuclear SNVs can be imaged indirectly through amplification by in situ PCR or CRISPR/Cas9-binding-mediated in situ rolling circle amplification followed by probe hydrization^{11, 12} However, the vast majority of genome imaging has been performed through direct hybridization, but to date, direct hybridization FISH with SNV sensitivity has not been realized. A single guide (sg) version of GOLDFISH (genome oligopaint via local denaturation FISH) was developed to address this technical gap.

### Methods

### Human Cell Lines

HEK293 human embryonic cells were purchased from the American Type Culture Collection (ATCC. CRL-1573) and cultured in DMEM with 4.5 g/L glucose, L-glutamine, and sodium pyruvate (Corning, 10-013-CV) supplemented with 10% heat inactivated fetal bovine serum (FBS, Corning 35-011-CV). Hutchinson-Gilford Progeria Syndrome (HGPS) fibroblasts were purchased from the Progeria Research Foundation and cultured in high glucose DMEM without L-glutamine (ThermoFisher, 11960-440) supplemented with 20% FBS (Corning, 35-011-CV), 1% Penicillin-Streptomycin (ThermoFisher, 15140-122) and 1% GlutaMAX (ThermoFisher, 35050-061). All cells were maintained at 37° C. in 5% CO₂ and imaging dishes were coated with 1 ug/cm2 fibronectin then air dried before plating.

Expression and Purification of Cas9 and Rep-X Cas9 nickase and eCas9 nickase were prepared as described previously with modifications²¹. Cas9 nickase was expressed using the pMJ826 plasmid (addgene, 39316). Mutagenesis was carried out to introduce the H840A mutation into eSpCas9(1.1) variant using pJSC114 plasmid (addgene, 101215) and QuikChange Lightning Site-Directed Mutagenesis Kit (Agilent, 210518). eCas9 nickase was expressed using the mutagenesis-modified pJSC114 plasmid. The plasmids were transformed into NEB BL21(DE3) competent cells (New England Biosciences). Cultures were maintained in Terrific Broth (Invitrogen) supplemented with 0.4% glycerol at 37° C. until induction at OD₆₀₀=0.5-0.6 at which point the temperature was lowered to 18° C. and cultures were induced with 0.5 mM IPTG (GoldenBio). Pellets were harvested after 16-18 h and resuspended in lysis buffer (50 mM Tris-HCl, 500 mM NaCl, 5% glycerol, 1 tablets per 50 ml protease inhibitor (EDTA-free, Roche), 0.2 mM PMSF, 1 mM TCEP, 1 mg/ml lysozyme, pH 7.5) and sonicated at 30% amplitude with 2 s on, 4 s duty cycle for 2 min, 3 times. Lysate was spun down and supernatant was mixed with 2 ml Ni-NTA resin (Qiagen) per 50 ml sample and incubated for 1 h at 4° C., then spun down and decanted. Resin was incubated with Wash Buffer (50 mM Tris-HCl, 500 mM NaCl, 10 mM imidazole, 5% glycerol, 1 mM TCEP, pH 7.5) at 4° C. for 5 min repeated 4 times then added to gravity column. Colum was then incubated with Elution Buffer (50 mM Tris-HCl, 500 mM NaCl, 1 mM TCEP, 300 mM imidazole, 5% glycerol, pH 8-8.5) and fractions were analyzed via denaturing PAGE. Samples were then desalted and concentrated using a 40 kD cut off filter into storage buffer (300 mM NaCl, 10 mM Tris-HCl, 0.1 mM EDTA, 50% glycerol, pH 7.5). Ni-NTA purification with desalting showed sufficient purity and activity for GOLD FISH applications and did not require further size selection chromatography.

Rep-X was prepared the same as previously described¹⁴. pET28a(+) with rep (C18L/C43S/C167V/C612A/S400C) was transformed into E. coli B21(DE3) (Sigma-Aldrich, CMC0014). A single colony was picked and grown in TB at 37° C. overnight, followed by 30° C. overnight. When OD reached the range between 0.3 and 0.4, the cells were moved to an 18° C. incubator. When OD reaches 0.6 to 0.8, the cells were induced expression with 0.5 mM IPTG and continue growth overnight. The cells were harvested by centrifugation for 15 min at 5000 rpm and 4° C. The pellet was resuspended in 40 ml of the lysis buffer (50 mM Tris-HCl pH 7.5, 5 mM Imidazole, 200 mM NaCl, 20% (w/v) sucrose, 15% (v/v) glycerol, 17.5 ug/ml PMSF, and 0.2 mg/ml Lysozyme) and sonicate to lyse the cells. The lysed cell mix was centrifuged at 14,000 rpm at 4° C. for 30-60 min and collect the supernatant. The supernatant was stir-mixed with pre-equilibrated Ni-NTA resin for 1.5 hours at 4° C. Ni-NTA purification was performed by washing the protein-bound resin with buffer A (50 mM Tris-HCl pH 7.5, 5 mM Imidazole, 150 mM NaCl, 25% (v/v) glycerol), followed by buffer A1M (50 mM Tris-HCl pH 7.5, 5 mM Imidazole, 1 M NaCl, 25% (v/v) glycerol) to remove any DNA residue, and final washed the protein-bound resin with buffer A, then eluted the Rep variant with imidazole buffer (50 mM Tris-HCl pH 7.5, 205 mM Imidazole, 150 mM NaCl, 25% (v/v) glycerol). 20 μM eluted Rep variant was mixed with 100 μM BMOE crosslinker to self-crosslink into Rep-X. The reaction was stir-mixed at room temperature for 1 hour. The excess crosslinker and Imidazole was removed by an overnight dialysis and stored in Rep-X storage buffer (50% glycerol, 600 mM NaCl, 50 mM Tris-HCl pH 7.5) at −80° C.

### Genome Sequences

The GRCh38.p13 Primary Assembly was used in this study and downloaded from NCBI. The coordinates of target loci are listed below:
MUC4-R region (Chr3: 195788656-195778790)
MUC4-NR region (Chr3: 195807684-195808777)
LMNA region (Chr1: 156137082-156138607)

### Sggoldfish Guide RNA and Probe Design

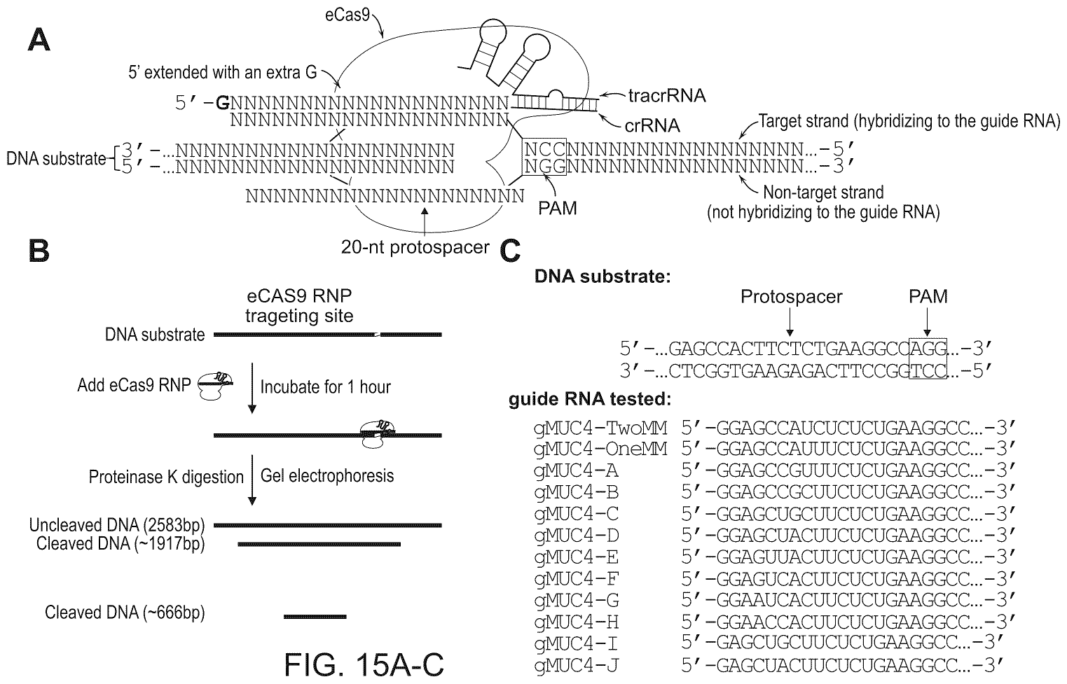

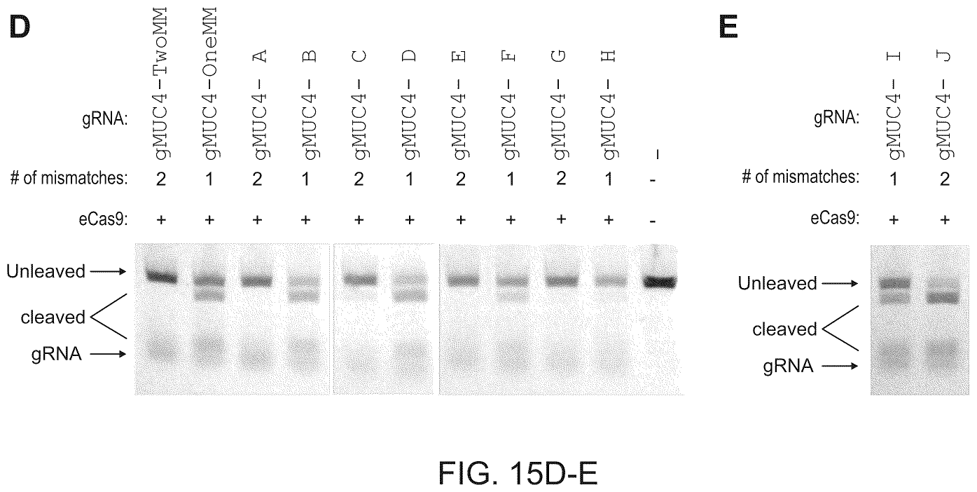

The SNV site should be within a protospacer of SpCas9. Because the previous study has demonstrated to target SNV at the PAM-proximal region¹¹, here we focused on testing SNVs located at PAM-distal region. The 13^{rd} to 18^{th} positions from the PAM are ideal (FIG. 15D). Because eCas9 can tolerate one PAM-distal mismatch, but two PAM-distal mismatches essentially inhibit cleavage under our conditions (FIGS. 15D, 16C, 20A and 20B), and additional mismatch was intentionally introduced into the guide RNA (e.g., the U at the 8^{th} position from the 5′ of crRNA in gMUC4-TwoMM and gMUC4-OneMM, FIG. 15C). Oligo FISH probes for sgGOLDFISH were designed using Oligoarray²². The target DNA sequence (˜1.5 kb) immediately following the target protospacer is input into the Oligoarray 2.1 with the following constraints: Length: 18- to 24-nt; Tm: 70° C. to 90° C.; % GC: 30-70; Max. Tm for structure: 54° C.; Min. Tm to consider X-hybrid: 54° C.; and there was no consecutive repeat of 5 or more identical nucleotides. Probes that can non-specifically bind to human genome, human noncoding RNA and E. coli tRNA were removed. The sequences of probes and are listed in Supplementary Table 2.

### Preparation of DNA and RNA

The designed oligo FISH probes (without any labeling/modification) were purchased from IDT, and fluorescently labeled as previously described²³. Briefly, to conjugate an amino-ddUTP at the 3′ end of each oligonucleotide, 66.7 μM DNA oligonucleotides, 200 μM Amino-11-ddUTP (Lumiprobe) and 0.4 U/μl Terminal Deoxynucleotidyl Transferase (TdT, Thermo Scientific, EP0162) were mixed in 1×TdT Reaction buffer (Thermo Scientific) and incubated overnight at 37° C. The reaction was cleaned up by ethanol precipitations and P4 beads (Bio-Rad, #1504124) spin columns. Next, the DNA oligonucleotides conjugated with amino-ddUTP were mixed with 100 μg of Cy3-NHS or Cy5-NHS (Lumiprobe or GE Healthcare) in 0.1 M sodium bicarbonate and incubated overnight at room temperature, and cleaned up by ethanol precipitations and P4 beads (Bio-Rad, #1504124) spin columns. Unlabeled oligonucleotides were removed by high-performance liquid chromatography (HPLC). The DNA substrates for in vitro cleavage assays are synthesized using Phusion® Hot Start Flex 2× Master Mix (NEB, M0536S) and purified using GeneJET PCR Purification Kit (Thermo Scientific, K0701). The primers are purchase from IDT and sequences are listed in Supplementary Table 2. For the guide RNA in FIG. 15C, crRNA was synthesized in vitro using HiScribe™ T7 Quick High Yield RNA Synthesis Kit (NEB, E2050S) according to the manufacturer's instructions, and purified by polyacrylamide gel electrophoresis. Alt-R® CRISPR-Cas9 tracrRNA (IDT) was purchase from IDT. The guide RNA was annealed by mixing crRNA and tracrRNA at 1:1 ratio in Nuclease Free Duplex Buffer (IDT), and incubating at 95° C. for 30 seconds, then slowly cooling down to room temperature over 1 hour. For other guide RNAs used in this study, the guide RNA was synthesized using EnGen® sgRNA Synthesis Kit, S. pyogenes (NEB, E3322V) according to the manufacturer's instructions. The template DNA sequences are listed in Supplementary Table 2.

### In Vitro Cleavage Assay

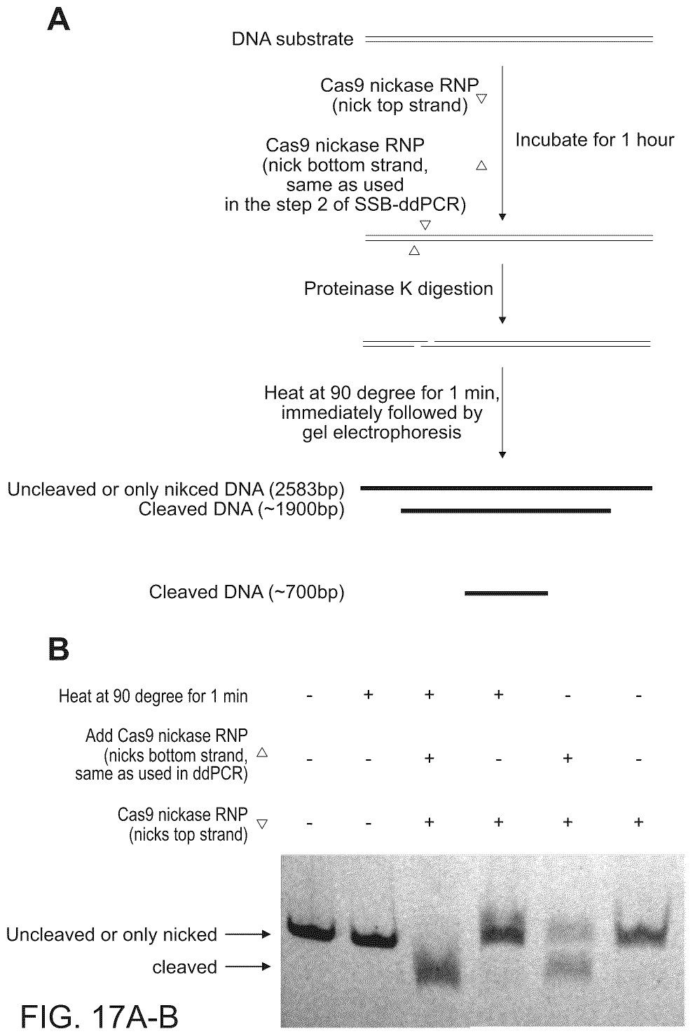

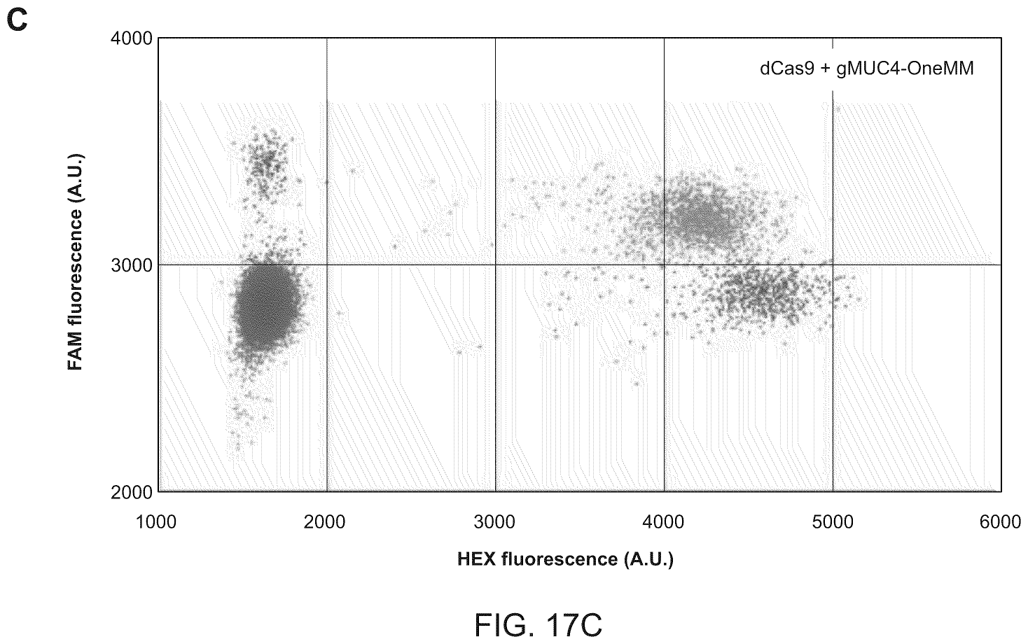

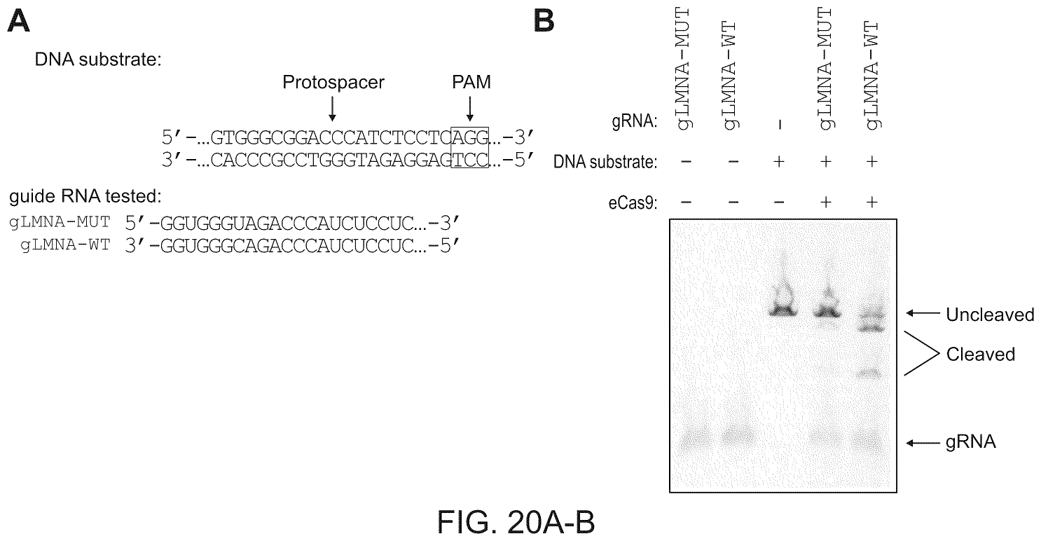

For FIGS. 15D, 20B, Cas9 RNP was assembled by mixing 200 nM eCas9 nickase with 400 nM guide RNA in the cleavage buffer (20 mM Hepes pH 7.5, 100 mM KCl, 7 mM MgCl₂, 5% (v/v) glycerol and 0.1% (v/v) TWEEN-20, freshly added 1 mM DTT), and incubated for 10 min at room temperature. Then 4 nM DNA substrate was added, and incubated at 37° C. for 1 hour. Next, 80 unites/mL of proteinase K (NEB, P8107S) was added to the reaction, and incubated at 37° C. for 30 min. The reaction was directly loaded into the agarose gel for electrophoresis. For FIG. 17B, 400 nM Cas9 nickase RNP cleaving the top strand and 400 nM Cas9 nickase RNP cleaving the bottom strand were assemble by mixing Cas9 nickase and corresponding guide RNA at 1:1 ratio and incubated for 10 min at room temperature. Then, 600 ng PCR-synthesized DNA substrate (FIG. 15C) was added to the mixture and incubated for 1 hour at 37° C. Next, 80 unites/mL of proteinase K (NEB, P8107S) was added to the reaction, and incubated at 37° C. for 30 min. The reaction was heated at 90° C. for 1 min to dissociate the two parts of the double-nicked DNA, followed by agarose gel electrophoresis.

### Cell Fixation

The HEK293T or HGPS cells adhered to the glass surface of an imaging dish were fixed at −20° C. for 15 min in pre-chilled MAA solution (methanol and acetic acid mixed at 1:1 ratio), then washed three times (5 min each wash at room temperature unless indicated) with PBS. The following steps were only performed in the SSB-ddPCR and the sgGOLDFISH in FIGS. 1b and 1c. The 0.1% pepsin in 0.1 M HCl was applied to the fixed HEK293T cells and incubated for 45 s at 37° C. The cells were washed with PBS once, and incubated in 70%, 90% and 100% EtOH at room temperature, each for 1 min. The cells were then washed three times with PBS.

### SSB-ddPCR

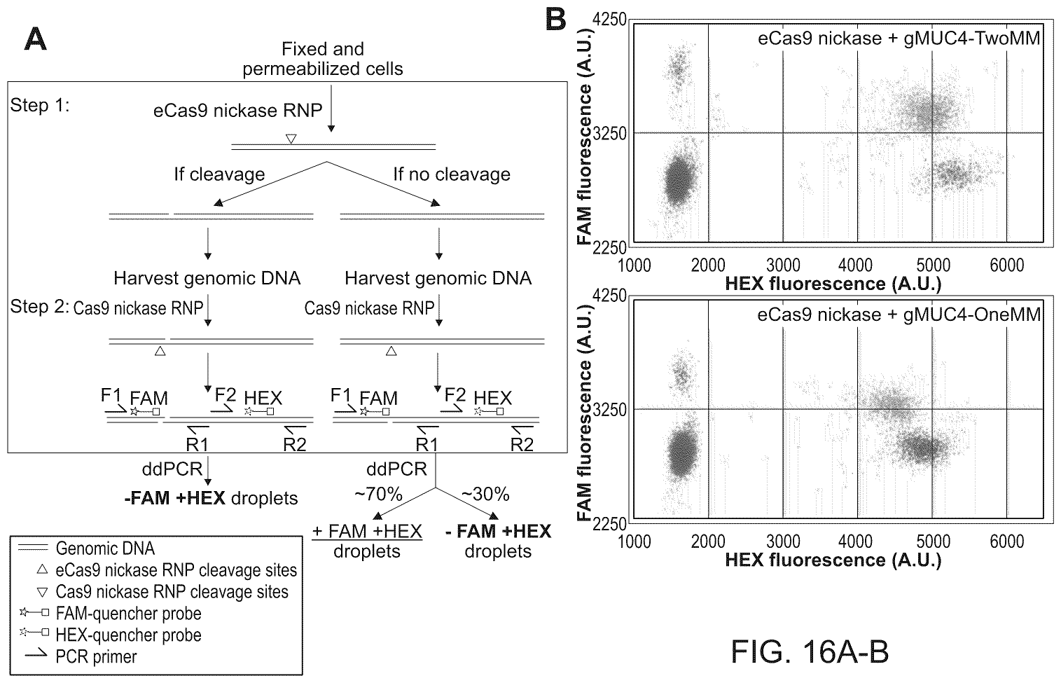

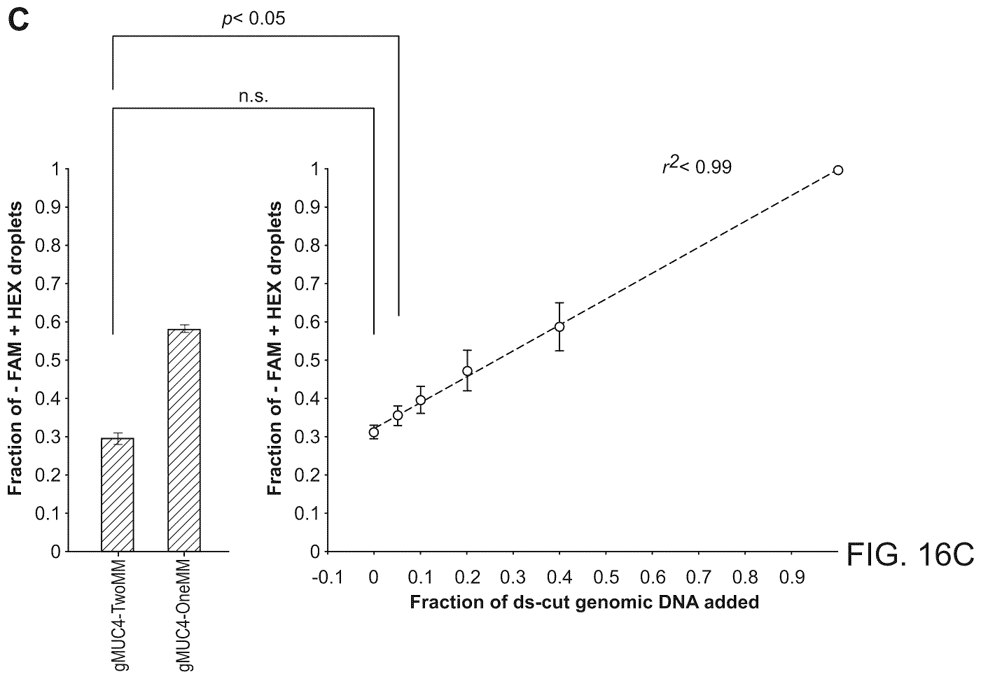

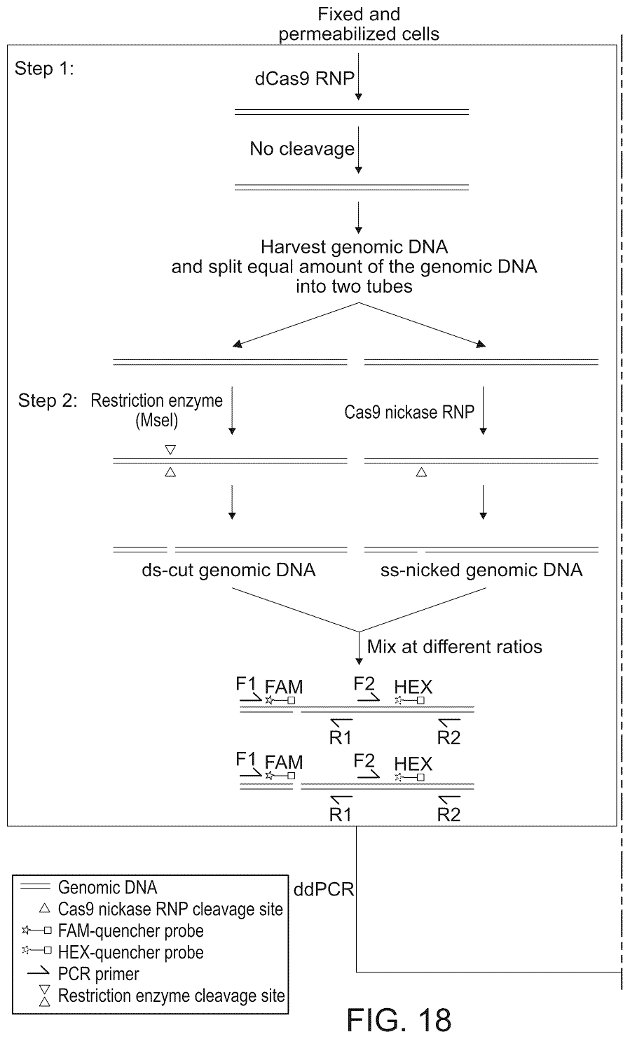

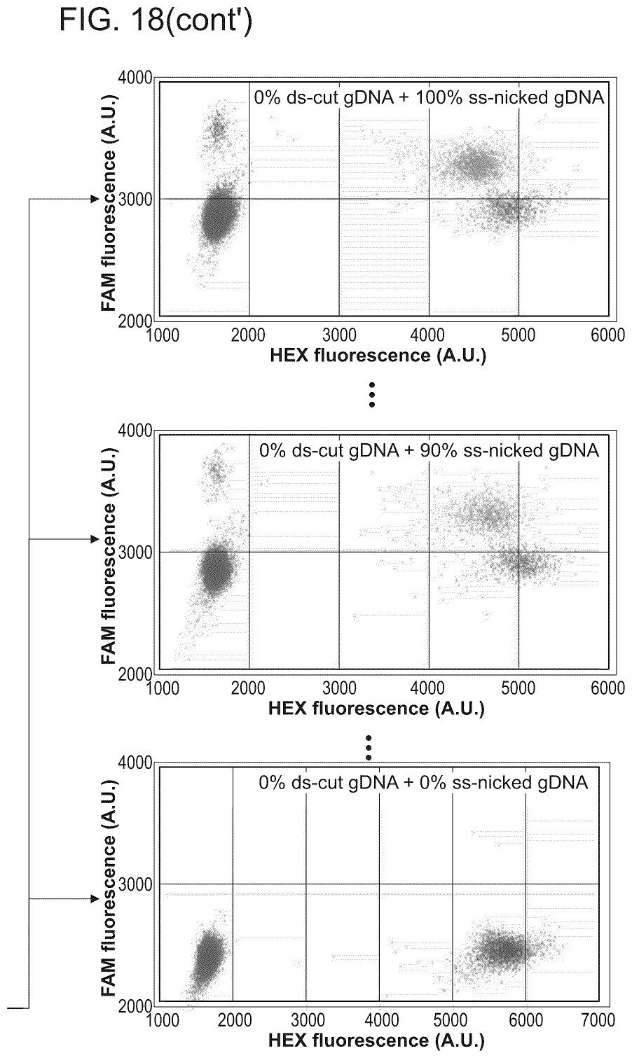

The SSB-ddPCR was performed similarly to DSB-ddPCR¹⁷ with modifications (FIG. 16A). The fixed and pepsin treated HEK293T cells adhered to the glass surface of the imaging dish were incubated in the binding-blocking buffer (20 mM Hepes pH 7.5, 100 mM KCl, 7 mM MgCl₂, 5% (v/v) glycerol and 0.1% (v/v) TWEEN-20, 1% (w/v) BSA, freshly added 1 mM DTT, freshly added 0.1 mg/ml E. coli tRNA) for 10 min at 37° C. Next, 100 nM eCas9 nickase was mixed with 200 nM gMUC4-TwoMM or gMUC4-OneMM in the binding-blocking buffer, and incubated for 10 min at room temperature. The 100 nM eCas9 nickase RNP was then applied to the cells, and incubated for 45 min at 37° C. After that, 2 mM ATP was supplied to the 100 nM eCas9 nickase RNP solution (i.e., the 100 nM eCas9 nickase RNP in the binding-blocking buffer supplied with 2 mM ATP), and incubated the cells in the solution for another 90 min at 37° C., followed by PBS wash 3 times. So far, these steps are the same as sgGOLDFISH in FIGS. 1b and 1c, expect that the Rep-X is omitted in SSB-ddPCR. To harvest genomic DNA from the cells, 60 mAU/mL proteinase K in PBS from DNeasy Blood & Tissue Kits (Qiagen, 69504) was applied to the cells and incubated for 30 min at 37° C. The solution was collected from the imaging dish, and genomic DNA was extracted using the DNeasy Blood & Tissue Kits by following manufacturer's protocol. The extracted genomic DNA (less than 8 ng/uL) was further treated with 400 nM Cas9 nickase RNP using the corresponding guide RNA in 1× NEBuffer r3.1 (NEB, B7203S) for 1 hour at 37° C. Next, 45 unit/mL proteinase K (NEB, P8107S) was added to the reaction and incubated for 30 min at 37° C. The genomic DNA was purified using Genomic DNA Clean & Concentrator-10 (Zymo, D4011) and eluted in water. Finally, 20 to 50 ng the genomic DNA was mixed with 250 nM probes, 900 nM primers and 250 unit/mL Eael (NEB, R0508S) in 1× ddPCR Supermix for Probes (no dUTP) (Bio-Rad, 1863023). Droplets were created using Droplet Generation Oil for Probes, DG8 Gaskets, DG8 Cartridges, and QX200 Droplet Generator (Bio-Rad); Droplets were transferred to a 96-well PCR plate and heat-sealed using PX1 PCR Plate Sealer (Bio-Rad). PCR amplification was performed with the following conditions: 95° C. for 10 min, 40 cycles of (94° C. for 30 sec, 55° C. for 30 sec, 72° C. for 2 min), 98° C. for 10 min, 12° C. hold. Droplets were then individually scanned using the QX200 Droplet Digital PCR system (Bio-Rad). To generate the standard curve, gMUC4-OneMM and dCas9 (instead of eCas9 nickase) was applied to the fixed and pepsin treated HEK293T cells as described above, and the genomic DNA was harvested (FIG. 18, Step 1). Half of the genomic DNA was treated with Cas9 nickase RNP as described above, which produces “ss-nicked genomic DNA”. Another half of the genomic DNA (less than 8 ng/uL) was treated with 0.2 unit/uL MseI (NEB, R0525S) for 1 hour at 37° C., and MseI was deactivated by incubating the reaction 20 min at 65° C. Following the deactivation, the MseI-treated genomic DNA was purified using Genomic DNA Clean & Concentrator-10 (Zymo, D4011) and eluted in water, which produces “ds-cut genomic DNA”. The “ss-nicked genomic DNA” and “ds-cut genomic DNA” were then mixed at different ratios for ddPCR as described.

sgGOLDFISH

The cells adhered to the glass surface of the imaging dish were incubated in the binding-blocking buffer (20 mM Hepes pH 7.5, 100 mM KCl, 7 mM MgCl₂, 5% (v/v) glycerol and 0.1% (v/v) TWEEN-20, 1% (w/v) BSA, freshly added 1 mM DTT, freshly added 0.1 mg/ml E. coli tRNA) for 10 min at 37° C. Next, 100 nM eCas9 nickase was mixed with 200 nM guide RNA in the binding-blocking buffer, and incubated for 10 min at room temperature. If the MUC4-R region was targeted, additional 20 nM eCas9 nickase and 40 nM gMUC4-R were also assembled in the binding-blocking buffer. After that, 2 mM ATP and 300 uM Rep-X was supplied to the 100 nM eCas9 nickase RNP solution (i.e., the 100 nM eCas9 nickase RNP in the binding-blocking buffer supplied with 2 mM ATP and 300 uM Rep-X), and incubated the cells in the solution for another 90 min at 37° C., followed by PBS wash 3 times. Next, RNase Cocktail™ Enzyme Mix (Invitrogen, AM2286) was diluted 100 times in PBS and incubated with the cells for 1 hour at 37° C. The cells were washed three times with PBS. The cells were then incubated in freshly made hybridization buffer (20% (v/v) formamide, 2× saline-sodium citrate (SSC), 0.1 mg/mL E. coli tRNA, 10% (w/v) dextran sulfate, 2 mg/mL BSA) for 10 min at room temperature. Fluorescently labeled oligo FISH probes (1 nM for MUC4-R, 2.5 nM per oligo FISH probe for MUC4-NR and LMNA, i.e., 57.5 nM and 90 nM final probe concentration for MUC4-NR and LMNA) in the hybridization buffer were applied to the cells and incubated for 1 hour at room temperature (repetitive targets) or 37° C. (non-repetitive targets). The cells were washed twice (10 min each wash) with wash buffer (25% formamide, 2×SSC) at 37° C. and once with PBS at room temperature for 5 min. One drop of Hoechst 33342 Ready Flow™ Reagent (Invitrogen, R37165) was mixed with 2 ml of PBS and incubated with the cells for 2 min at room temperature. Finally, imaging buffer (2×SSC and saturated Trolox (>5 mM), 0.8% (w/v) dextrose) supplied with GLOXY (1 mg/ml glucose oxidase, 0.04 mg/ml catalase) was added to the cells for imaging.

### Immunofluorescence

For progerin immunofluorescence, the cells after sgGOLDFISH were incubated in IF buffer (1× Blocker™ BSA in PBS (Thermo Scientific, 37525) supplied with 0.1% Tween-20) at room temperature for 20 min. Progerin Monoclonal Antibody (13A4) (Thermo Scientific, 39966) was diluted 500 times in the IF buffer, and applied to the cells for overnight incubation at 4° C. The cells were washed three times with PBS, and incubated with 500 times diluted Alexa750-labeled secondary antibody (Invitrogen, A-21037) in the IF buffer for 30 min at room temperature. Finally, the cells were wash 3 times with PBS and imaged in the imaging buffer. For Lamin A/C immunofluorescence, the cells after fixation were incubated in IF buffer (1× Blocker™ BSA in PBS (Thermo Scientific, 37525) supplied with 0.1% Tween-20) at room temperature for 20 min. Anti-Lamin A+Lamin C antibody [4C11](Abcam, ab238303) was diluted 500 times in the IF buffer, and incubated with the cells for 1 hour at room temperature. The cells were washed three times with PBS, and incubated with 500 times diluted Alexa750-labeled secondary antibody (Invitrogen, A-21037) in the IF buffer for 30 min at room temperature. Finally, the cells were wash 3 times with PBS and imaged in the imaging buffer.

### Microscopy

sgGOLDFISH imaging was performed using Nikon Eclipse Ti microscope equipped with Nikon perfect focus system, Xenon arc lamp. The system was driven by Elements software. Nikon 60×/1.49 NA objective (CFI Apo TIRF) was used. Emission was collected using a custom laser-blocking notch filter (ZET488/543/638/750M) from Chroma. Images were recorded using an electron-multiplying charge-coupled device (Andor iXon 888). Images were recorded as z-stacks (20 to 30 steps), with 300 nm to 500 nm step size.

### Dna-Free Base Editing in HGPS Fibroblasts

The guide RNA for base editor to correct the HGPS mutations (LMNA-VRQRABE-sgRNA) was purchased from IDT (see Table 2 for sequence). To prepare DNA template for VRQR-ABE7.10max mRNA, pUC19 (NEB, N3041S) was linearized using EcoRI-HIF (NEB, R3101S) and HindIII-HF (NEB, R3104S). VRQR-AEB fragment was PCR-synthesized using the Plasmid #154429 (addgene) and VRQR-AEB-primer-F and VRQR-AEB-primer-R (see Supplementary Table 2 for primer sequences), and gel purification was carried out to remove non-specific products. Mutagenesis was performed using pcDNA3.3-eGFP (addgene, Plasmid #26822) and T7-Mutagenesis-primer-F and T7-Mutagenesis-primer-R to replace the “G” following the T7 promoter sequence with an “A” by the QuikChange Lightning Site-Directed Mutagenesis Kit (Agilent, 210518). T7-5′UTR fragment was PCR-synthesized using the mutagenesis-modified pcDNA3.3-eGFP and T7-5′UTR-primer-F and T7-5′UTR-primer-R. The 3′UTR fragment was PCR-synthesized using the mutagenesis-modified pcDNA3.3-eGFP and 3′UTR-primer-F and 3′UTR-primer-R. Next, the linearized pUC19, VRQR-AEB fragment, T7-5′UTR fragment, 3′UTR fragment was assembled into a plasmid (VRQRABE-mRNA plasmid) using NEBuilder HiFi DNA Assembly Master-Mix (UEB, E2621 S) according to manufacturer's protocol. The linear VRQRABE-mRNA DNA template was PCR-synthesized using VRQRABE-mRNA plasmid, VRQRABE-mRNA-linearTemplate-F and VRQRABE-mRNA-linearTemplate-R. All PCR reactions were performed using Q5® Hot Start High-Fidelity 2× Master Mix (NEO, M494S). The in vitro transcription of VRQR-ABE7.10max mRNA reaction contains 50 ng/uL linearized VRQRABE-mRNA DNA template, ATP/CTP/GTP (5 mM each), 5 mM N1-methylpseudouridine (TriLink, N-108-1), 4 mM CleanCap AG (TriLink, N-7113), 1 unit/uL Murine RNase Inhibitor (NEB, M0314S), 0.002 units/uL Yeast inorganic Pyrophosphatase (NEB, M2403S) and 8 units/uL T7 RNA Polymerase (NEB, M0251S) in the transcription buffer (40 mM Tris-HCl pH 8, 20 mM spermidine, 0.02% (v/v) Triton X-100, 165 mM magnesium acetate, freshly added 10 mM DTT). The in vitro transcription reaction was incubated at 37° C. for 2 hours, and treated with DNase I by supplying with 1× DNase buffer (NEB, B0303S) and 0.3 units/uL DNase I (M0303S) and incubating at 37° C. for 20 min. The reaction was purified using Megaclear™ Transcription Clean-Up Kit (Invitrogen, AM1908), and dephosphorylated using 0.25 units/uL Antarctic Phosphatase (NEB, M0289S) according to manufacturer's protocol. The VRQR-ABE7.10max mRNA was purified again using the Megaclear™ Transcription Clean-Up Kit. All electroporation experiments were carried out using the Lonza 4D-Nucleofector System. For mRNA editing in HGPS cells, 5 μg of LMNA-VRQRABE-sgRNA was mixed in a total 25 uL volume (SE kit, Lonza) then resuspended with 200k HGPS cells and electroporated using the with CM-120 setting. Cells were maintained at 37° C. in 5% CO₂ for 3 days before collecting genomic DNA using DNeasy Blood & Tissue Kits (Qiagen, 69504) and sequencing.

### Data Analysis for GOLD FISH

Images were processed using Fiji/ImageJ. Z-stack images were projected to a single plane using the ‘Max Intensity’ Z-Projection function. The contrasts of images were linearly adjusted by changing the minimum and maximum values using the ‘brightness/contrast’ function in ImageJ for optimal visualization purpose only. FISH-quant was used to find foci in each cell and fitted with three-dimensional (3D) Gaussian function²⁴. The nuclear edge, nuclear area and the distance from a FISH focus to the nuclear edge were analyzed using custom-written MATLAB scripts.

### Results

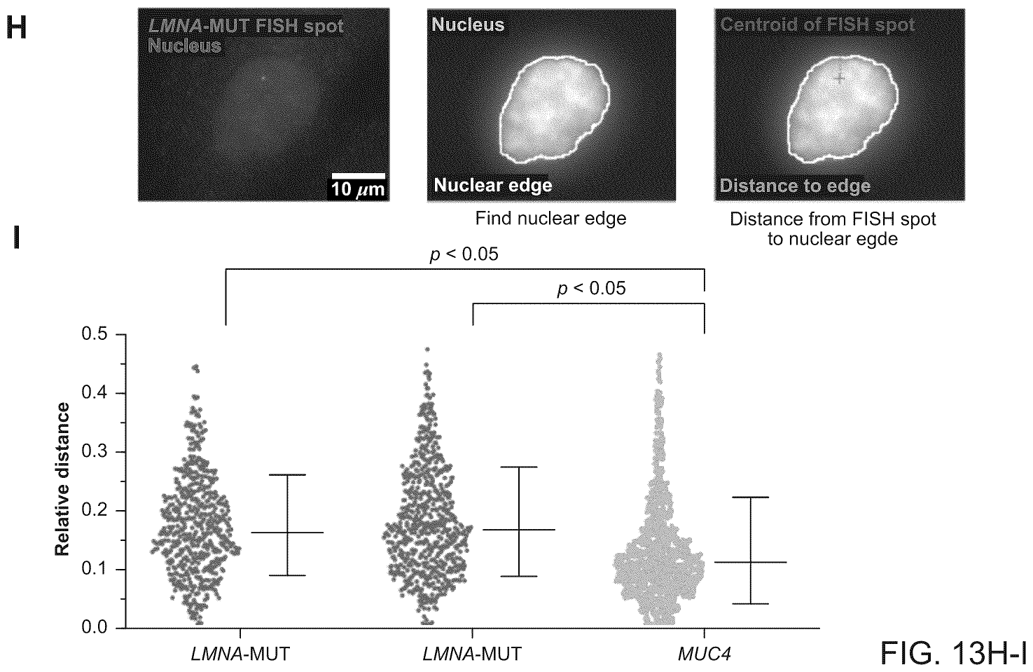

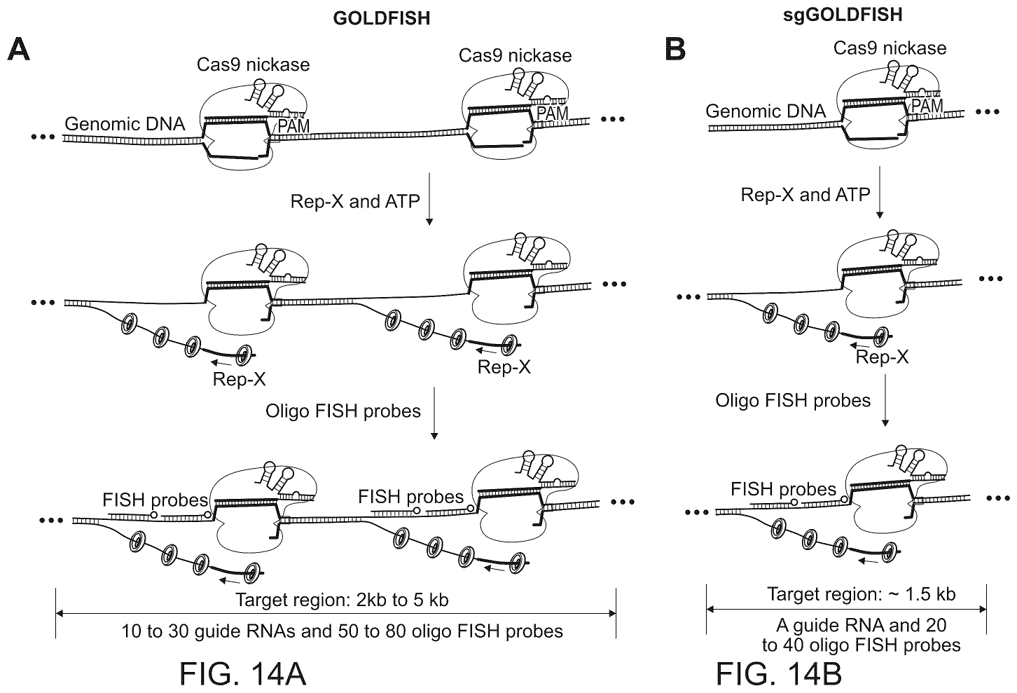

The original GOLDFISH method used multiple guide RNAs tiling a genomic region of interest in complex with Cas9 nickase (SpCas9 with H840A mutation¹³) to cleave genomic DNA at multiple sites (FIG. 14A)¹⁴. This allowed local denaturation of targeted genomic DNA by loading an engineered helicase, Rep-X, to the cleaved strands so that DNA downstream is unwound to expose binding sites for FISH probes. Because only several kilobases of DNA unwound, GOLDFISH greatly reduces nonspecific binding of FISH probes to other genomic regions compared to conventional FISH that globally denatures genomic DNA. Because GOLDFISH requires Cas9 cleavage which is much more sequence-stringent compared to Cas9 binding, it also has superior signal to background ratio compared to methods that rely on Cas9 binding¹⁴.

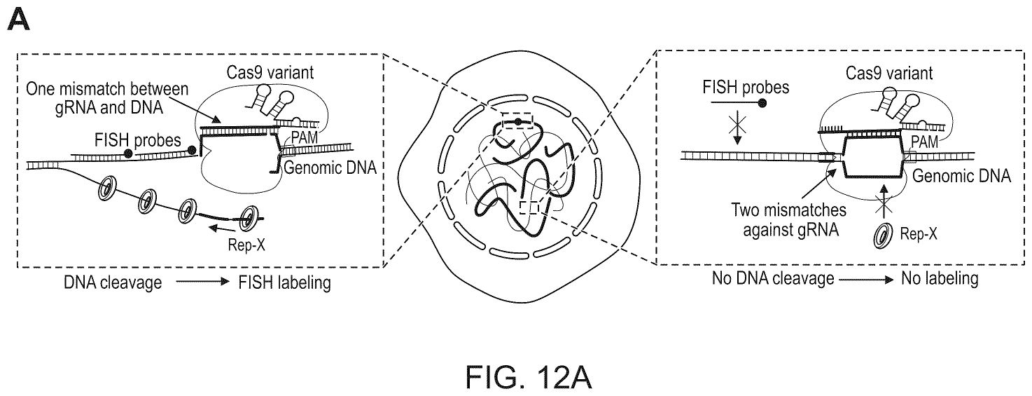

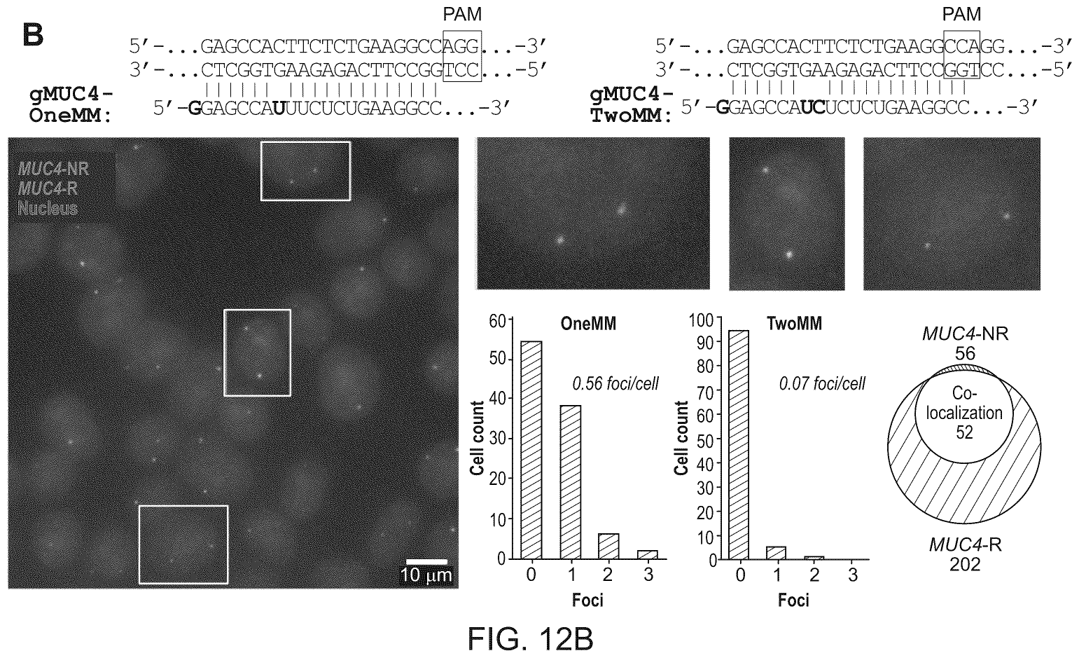

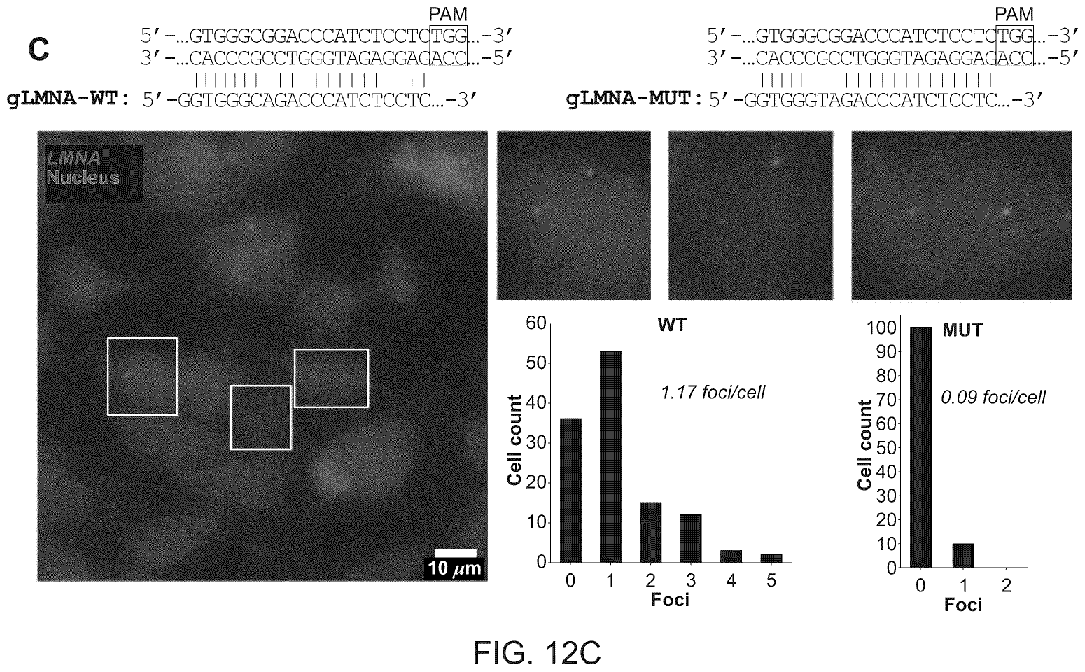

The use of multiple cut sites in GOLDFISH enabled high efficiency labeling even if the cleavage efficiency at a single site is not very high. But multiple cut sites made it impossible to make FISH labeling sensitive to an SNV. It was hypothesized that, instead of using multiple guide RNAs, GOLDFISH using a single guide RNA (hence called sgGOLDFISH, FIG. 14B) may achieve SNV sensitivity if the Cas9 cleavage activity is optimized to be SNV-sensitive (FIG. 12A). If so, by rationally designing guide RNA and choosing engineered Cas9 variant that has higher cleavage specificity, sgGOLDFISH can preferentially label one of the two alleles even when the two alleles differ by only a single nucleotide (FIG. 12A). To test this concept, a Cas9 ribonucleoprotein (RNP) variant was assembled by combining the specificity-improving mutations in eSpCas9(1.1)¹⁵ with a 5′ extended guide RNA¹⁶ (hereinafter called eCas9 RNP, which cleaves both the target and nontarget strands (FIG. 15A), and tested its cleavage activity using an in vitro cleavage assay (FIGS. 15B, 15C). Efficient cleavage was observed for 4 out of 5 guide RNAs with 1 PAM-distal mismatch, but no cleavage activity for five guide RNAs containing 2 PAM-distal mismatches (FIG. 15D). Such specificity was not observed with canonical guide RNA in complex with eCas9 (FIG. 15E). These data indicate that adding an extra PAM-distal mismatch can drastically reduce the cleavage activity of eCas9 RNP.

Cleaving only the non-target strand by Cas9 nickase is sufficient for Rep-X unwinding the downstream genomic DNA and FISH probe hybridization in GOLDFISH¹⁴. The eCas9 (H840A) variant (hereinafter called eCas9 nickase)¹³ was therefore created, and its cleavage activity in fixed cells was measured (note that in GOLDFISH, cleavage and subsequent steps are performed in fixed cells). The fraction of double-strand breaks (DSBs) at a target site in the cell population can be measured using a droplet digital PCR (ddPCR) assay¹⁷. ddPCR, however, is not sensitive to single-strand breaks (SSBs). Here the ddPCR assay is extended through an additional nicking step to make it SSB-sensitive (FIGS. 16A-16C, 17A-17C, 18). Using this assay with the eCas9 nickase in complex with a guide RNA that contains two mismatches against the MUC4 gene (gMUC4-TwoMM), or a guide RNA with one mismatch (gMUC4-OneMM) (FIG. 15C), <5% and ˜40% DNA was observed being cleaved, respectively (FIGS. 16B, 16C). These data demonstrate that an extra mismatch drastically reduced the cleavage efficiency in fixed cells so that a contrast of about an order of magnitude can be obtained.

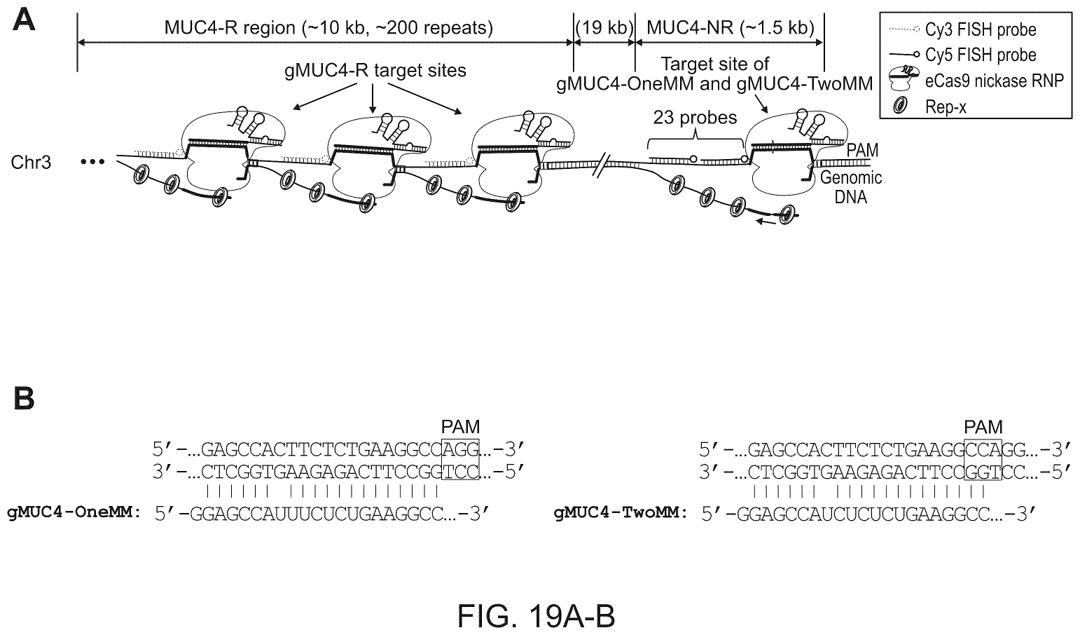

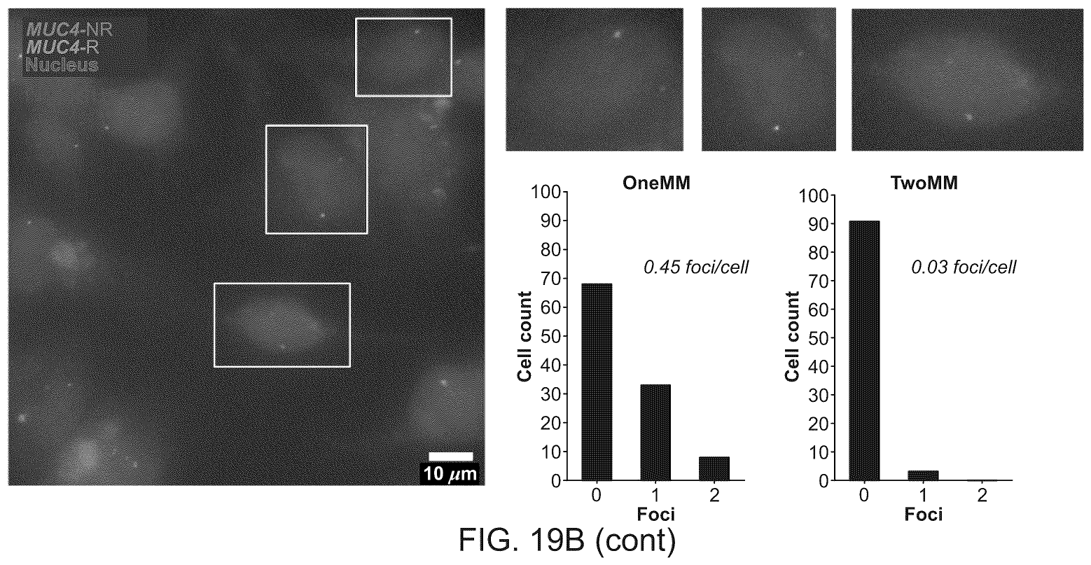

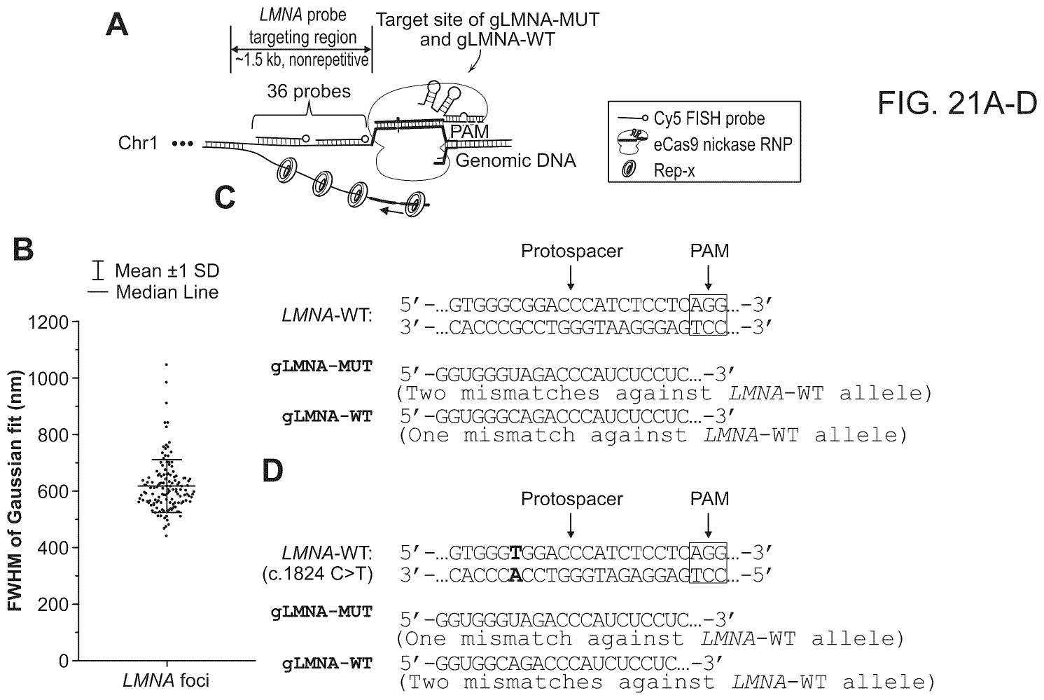

sgGOLDFISH was first tested in proteinase-treated cells (HEK293T) using the eCas9 nickase complexed with the gMUC4-OneMM or gMUC4-TwoMM and 23 Cy5-labeled FISH probes against a 1.5-kb non-repetitive region in the MUC4 gene (MUC4-NR) adjacent to the target protospacer (FIG. 19A). Another guide RNA (gMUC4-R) and a Cy3-labeled FISH probe were also designed against a repetitive region (MUC4-R) 19-kb from the MUC4-NR region to evaluate the specificity and sensitivity of sgGOLDFISH (FIG. 19A). With gMUC4-OneMM, on average 0.56 MUC4-NR FISH foci per cell (foci/cell) were detected (FIG. 12B). With gMUC4-TwoMM, on average 0.07 foci/cell were detected (FIG. 12B). With concurrent use of gMUC4-R and gMUC4-OneMM, we found 52 of 56 MUC4-NR foci colocalized with MUC4-R foci, indicating high labeling specificity of sgGOLDFISH (FIG. 12B). There were a total of 202 MUC4-R foci among which 52 showed colocalization with MUC4-NR, suggesting about 25% detection efficiency of sgGOLDFISH. The sgGOLDFISH experiment was repeated without the proteinase treatment and 0.45 foci/cell was observed for gMUC4-OneMM and 0.03 foci/cell was observed for gMUC4-TwoMM, again demonstrating SNV-sensitivity (FIG. 19B). sgGOLDFISH was also performed against the LMNA gene using doubly mismatched (gLMNA-MUT) or singly mismatched (gLMNA-WT) guide RNA (FIGS. 20A, 21A). 1.17 foci/cell was observed for gLMNA-WT and 0.09 foci/cell for gLMNA-MUT (FIG. 12C). These data show that a single-nucleotide difference can result in about an order of magnitude difference in sgGOLDFISH detection efficiency. The full width at half maximum of Gaussian fit to the imaged LMNA foci is 617±92 nm (mean±SD, FIG. 21), indicating sgGOLDFISH provides well-defined spots suitable for subnuclear localization analysis.

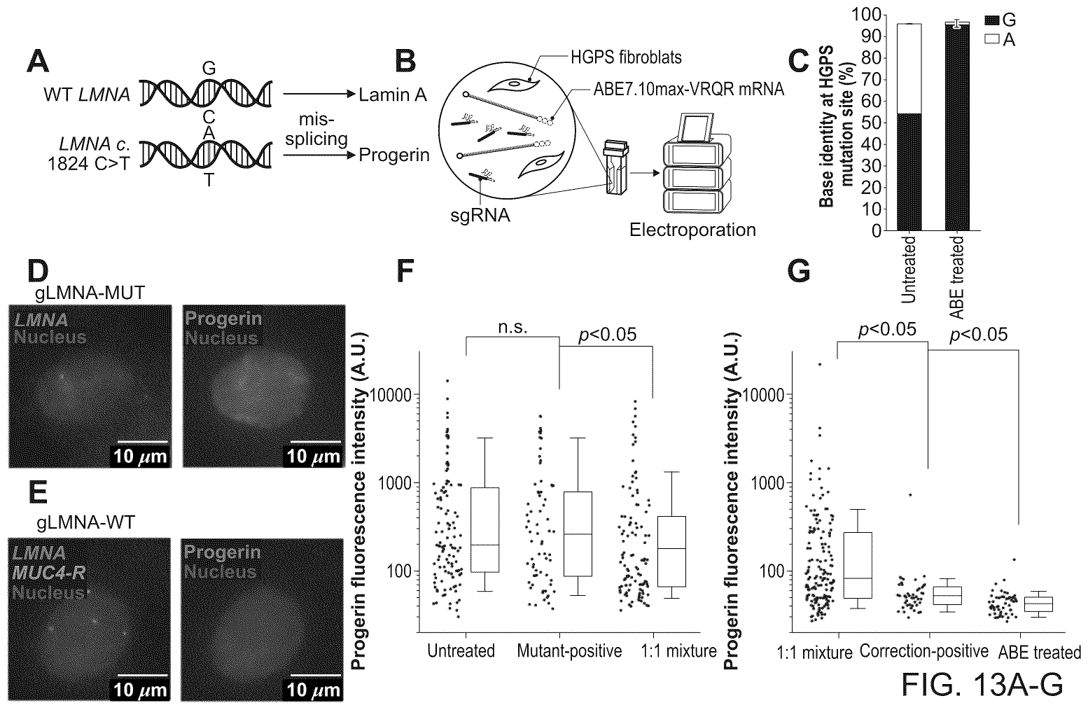

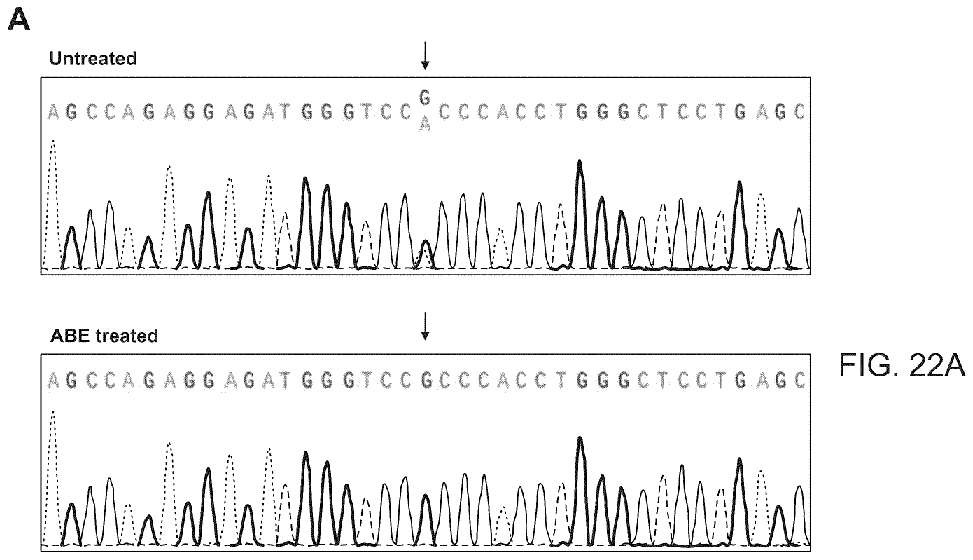

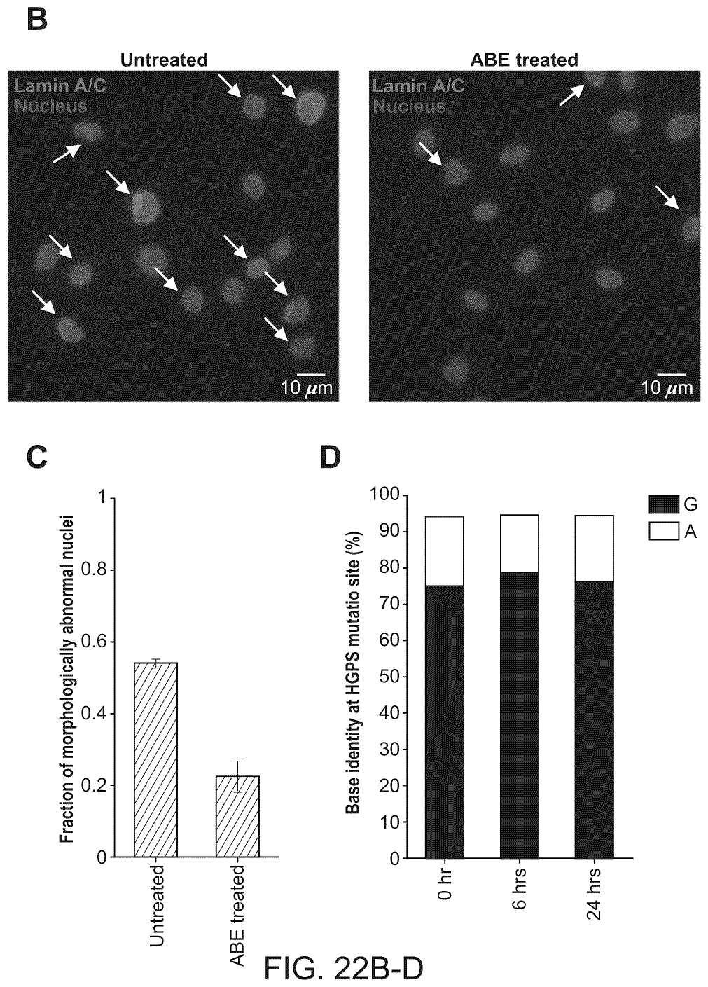

sgGOLDFISH was next applied in fibroblasts derived from the Hutchinson-Gilford progeria syndrome (HGPS) patient. The HGPS cell has one copy of normal LMNA gene (LMNA-WT), and one copy of mutated LMNA gene (LANA-MUT) that carries a point mutation (c. 1824 C>T) (FIG. 13A), which causes expression of progerin, a truncated gene product, and alterations of nuclear shape¹⁸. The gLMNA-MUT guide RNA described above has two mismatches against the wild-type LMNA sequence and one mismatch against the progeria mutant sequence (FIG. 21C) and gLMNA-WT has one mismatch against the wild-type and two mismatches against the mutant (FIG. 21D). Therefore, sgGOLDFISH using gLMNA-MUT should preferentially label the mutant allele whereas the wild type allele is preferentially labeled when gLMNA-WT is used (FIG. 12A). To test this prediction, HGPS mutation-corrected fibroblasts were created by delivering adenine base editor ABE7.10max-VRQR (ABE) mRNA and corresponding sgRNA into the HGPS cells¹⁹ (FIG. 13B). This DNA-free approach efficiently corrected the HGPS mutation (>94% efficiency) without the risk of unwanted DNA integration into the genome (FIGS. 13C, 22A). Consistently, the fraction of morphologically abnormal nuclei was significantly reduced after the ABE treatment (FIGS. 22B, 22C).

In order to test if sgGOLDFISH preferentially labels the progeria mutant allele with gLMNA-MUT, a cell mixture that contains 50% uncorrected HGPS cells and 50% ABE-corrected HGPS cells was made (hereinafter called 1:1 mixture, FIG. 22D). sgGOLDFISH against the LMNA gene using gLMNA-MUT was applied to the 1:1 mixture in parallel with progerin immunofluorescence, and a cell with at least one LMNA sgGOLDFISH spot was assigned as a “mutant-positive cell” (FIGS. 13D, FIG. 23A). The progerin immunofluorescence intensity averaged over the nucleus was comparable between the mutant-positive cells and untreated HGPS cells, but was significantly lower for cells randomly selected from the 1:1 mixture (FIG. 13F), consistent with reduced progerin expression after the HGPS mutation correction¹⁹. Therefore, sgGOLDFISH successfully identified uncorrected HGPS cells from a mixed population.

Next, sgGOLDFISH was performed in the 1:1 mixture again but using gLMNA-WT instead (FIGS. 13E, 23B). In parallel, GOLDFISH was performed against the MUC4-R region to estimate the cell cycle stage at the same time (FIG. 13E, e.g., detection of two MUC4-R foci indicates G0/G1). It was previously shown that the GOLDFISH detection efficiency of the MUC4-R region in fibroblasts is very high (around 90%)¹⁴. An ABE-corrected HGPS cell should have 2 to 4 copies of LMNA-WT alleles, depending on cell cycle. Therefore, cells with two LMNA foci and two MUC4-R foci, or four LMNA foci and four MUC4-R foci, were assigned as “correction-positive cells” (FIG. 13E). The progerin fluorescence was significantly lower for the correction-positive cells than for cells randomly selected from the 1:1 mixture (FIG. 13G). Taken together, these data provide evidence that sgGOLDFISH can preferentially label the LMNA-WT or the LMNA-MUT alleles even though these two alleles only differ by a single base pair, and that sgGOLDFISH can be used to identify base-edited cells or unedited cells from a heterogeneous population. It was noted that the progerin fluorescence is slightly higher for the correction-positive cells than for ABE-treated HGPS fibroblasts, probably because a small fraction of the LMNA-MUT alleles were labeled giving false positives (FIG. 13G).

Direct hybridization of probes allows for accurate localization of sequences of interest. To demonstrate sgGOLDFISH for sub-nuclear spatial analysis, the distance of LMNA and MUC4 foci to the nuclear edge in HGPS fibroblasts was measured. The MUC4 foci are closer to the nuclear edge than the LMNA foci (FIGS. 13H, 13I), consistent with Lamin A/C-ChIP (chromatin immunoprecipitation) data from the same HGPS line which showed stronger ChIP signal, which is a measure of proximity to the nuclear membrane, for MUC4 compared to LMNA (FIG. 23C)²⁰.

Discriminating the LMNA-MUT allele from the LMNA-WT allele requires sgGOLDFISH to distinguish the G-U wobble base pair from A-U base pair (FIGS. 21C, 21D), which has been shown to be difficult using oligonucleotide probes alone¹⁰. Moreover, existing methods visualizing SNVs in nuclear DNA involve proteinase treatment that excludes concurrent immunofluorescence and unavoidably perturbs nuclear architecture^{11, 12}. In contrast, proteinase treatment is not necessary for sgGOLDFISH. Although the cleavage activity of Cas9 is dependent on local sequence, it can be fine-tuned through guide RNA by adding/removing intentionally introduced mismatches or the 5′ extra guanine for new targets¹⁶. Overall, given the single-nucleotide sensitivity, immunofluorescence compatibility, the ability to accurately localize SNVs, and relatively broad SNV targeting scope (see Table 3 for comparison with other methods), sgGOLDFISH will be of value for researchers to study, for example, point mutation-related diseases or detect precise genome editing such as base editing.

Table 3|Comparison of sgGOLDFISH with other nuclear DNA FISH methods. CasPLA relies on Cas9's binding specificity to discriminate SNVs, therefore limits the target SNVs within a protospacer and proximal to (<10 bp) the protospacer adjacent motif (PAM)¹. sgGOLDFISH relies eCas9 nickase's cleavage specificity to discriminate SNVs, hence allows for targeting SNVs distal to PAM. STAR-FISH is based on in situ PCR that produces cloud-like signals which reduces the localization accuracy of target SNVs², whereas in sgGOLDFISH probes directly hybridize to genome and produce well-defined signals. Furthermore, CasPLA and STAR-FISH requires proteinase treatment to detect nuclear SNVs^{1,2}, while sgGOLDFISH does not require proteinase treatment. Zombie is limited to detect SNVs (e.g., SNVs generated by base editor) in pre-integrated DNA barcodes because it requires phage promoters upstream of the target SNV³. In contrast, there is no need to modify the genome of samples for sgGOLDFISH.

| | Nuclear DNA FISH methods | | | | | |
|---|---|---|---|---|---|---|
| | | | | | Conventional | Live cell Cas9 |
| | sgGOLDFISH | CasPLA¹ | STAR-FISH² | Zombie³ | DNA FISH⁴ | imaging⁵ |
| Targeting scope of | PAM-distal | PAM- | 3′ end of a | Pre-integrated | Not SNV | Not SNV |
| nuclear SNV | (Yes) | proximal | PCR primer | DNA barcodes | sensitive | sensitive |
| | PAM-proximal | | | with phage | | |
| | (Possible) | | | promoters | | |
| Labeling efficiency of | Moderate | Moderate | High | High | High | Moderate |
| non-repetitive | | | | | | |
| targets | | | | | | |
| Global DNA | Not Required | Not Required | Required | Not Required | Required | Not Required |
| denaturation | | | | | | |
| Protein | Yes | No | No | Yes | Yes | No |
| immunofluorescence | | | | | | |
| compatible | | | | | | |
| Live cell imaging | No | No | No | No | No | Yes |
| Tissue imaging | Possible | Yes | Yes | Yes | Yes | Possible |

### REFERENCES

1. Visscher, P. M. et al. The American Journal of Human Genetics 101, 5-22 (2017).
2. Larsson, C., Grundberg, I., Söderberg, O. & Nilsson, M. Nature methods 7, 395-397 (2010).
3. Levesque, M. J., Ginart, P., Wei, Y. & Raj, A. Nature methods 10, 865-867 (2013).
4. Deng, R. et al. Angewandte Chemie International Edition 53, 2389-2393 (2014).
5. Kühnemund, M. et al. Nature Communications 8, 13913 (2017).
6. Mellis, I. A., Gupte, R., Raj, A. & Rouhanifard, S. H. Nature methods 14, 801-804 (2017).
7. Marras, S. A. E., Bushkin, Y. & Tyagi, S. Proceedings of the National Academy of Sciences 116, 13921-13926 (2019).
8. Ke, R. et al. Nature methods 10, 857-860 (2013).
9. Larsson, C. et al. Nature methods 1, 227-232 (2004).
10. Askary, A. et al. Nature biotechnology 38, 66-75 (2020).
11. Zhang, K. et al. J Am Chem Soc 140, 11293-11301 (2018).
12. Janiszewska, M. et al. Nature genetics 47, 1212-1219 (2015).
13. Jinek, M. et al. Science 337, 816 (2012).
14. Wang, Y. et al. Molecular Cell (2021).
15. Slaymaker, I. M. et al. Science 351, 84 (2015).
16. Okafor, I. C. et al. Nucleic Acids Res 47, 11880-11888 (2019).
17. Rose, J. C. et al. Nature methods 14, 891-896 (2017).
18. Goldman, R. D. et al. Proceedings of the National Academy of Sciences 101, 8963-8968 (2004).
19. Koblan, L. W. et al. Nature 589, 608-614 (2021).
20. Mccord, R. P. et al. Genome Research 23, 260-269 (2013).
21. Wang, Y. et al. Proceedings of the National Academy of Sciences 118 (2020).
22. Rouillard, J. M. Nucleic Acids Research 31, 3057-3062 (2003).
23. Gaspar, I., Wippich, F. & Ephrussi, A. RNA (2017).
24. Mueller, F. et al. Nature methods 10, 277-278 (2013).

### Example 4: SSB-ddPCR Assay

Sequencing-based methods have been developed for mapping single-strand breaks (SSBs) or double-strand breaks (DSBs) in cells, but they are expensive and do not provide the absolute value of the fraction of DNA carries the breaks at a target site in the cell population⁶⁻¹⁰. A previously developed droplet digital PCR (ddPCR) assay unbiasedly measures the fraction of DNA with DSBs at a target site, but it is insensitive to SSBs. To quantitatively measure the cleavage efficiency of eCas9 nickase in complex with gMUC4-OneMM or gMUC4-twoMM in fixed cells, the ddPCR assay was modified to make it SSB-sensitive (therefore we call the modified ddPCR assay as SSB-ddPCR assay). The key modification is that the SSB was converted DSB by an additional Cas9 nickase treatment.

In the Step 1 of the SSB-ddPCR assay, eCas9 nickase with gMUC4-OneMM or gMUC4-TwoMM was applied to fixed and permeabilized HEK293T cells to cleave its target genomic DNA, which would introduce a SSB at one of the DNA strands if cleavage occurs (FIG. 16A, a SSB is introduced at the top strand if cleavage occurs). The cells were then treated with proteinase K and the genomic DNA was harvested. In the Step 2, Cas9 nickase RNP (400 nM) which cleaves the other strand (i.e., different from the scissile strand in Step 1) was mixed with less than 600 ng of the purified genomic DNA (FIG. 16A, a SSB is introduced in the bottom strand in the Step 2). The efficiency of the Cas9 nickase RNP to cleave the bottom strand was found to be around 100% under the experimental conditions herein (FIGS. 17A, 17B). Finally, after removing the Cas9 nickase RNP from the Step 2, the genomic DNA was mixed with two pairs of primers and two probes for ddPCR (FIG. 16A). The F1/R1 primers span the cleavage sites of the eCas9 nickase RNP in the Step 1 and the Cas9 nickase RNP in the Step 2, while the F2/R2 primers do not (FIG. 16A). The F1/R1 and F2/R2 amplicons spaced by 216 base pairs. The amplification of the F1/R1 and F2/R2 amplicons are detected using FAM-quencher probe and HEX-quencher probe, respectively. In the ddPCR reactions, the DNA polymerase digests the probe annealed to template DNA by using its proofreading exonuclease activity, and releases the fluorescent dye from the quencher¹¹. Therefore, a droplet shows FAM fluorescence or HEX fluorescence indicates amplification of the F1/R1 amplicon or the F2/R2 amplicon, respectively (FIG. 16A). Hereinafter droplets with negative FAM signal and positive HEX signal were referred to as “−FAM+HEX droplets” (FIG. 16B green spots), and the droplets with positive FAM signal and positive HEX signal as “+FAM+HEX droplets” (FIG. 16B, orange spots). The fraction of “−FAM+HEX droplet” was calculated by using the number of “−FAM+HEX droplets” divided by the total number of “−FAM+HEX droplets” and “+FAM+HEX droplets” (FIGS. 16B, 16C). When gMUC4-TwoMM, which contains two mismatches against the target, or gMUC4-OneMM, which contains one mismatch, was complexed with eCas9 nickase for the SSB-ddPCR assay in HEK293T cells, we observed the fractions of “−FAM+HEX droplets” were 0.283±0.005 and 0.580±0.010, respectively (FIGS. 16B, 16C).

Ideally, if there is no cleavage by eCas9 nickase RNP in the Step 1, the input DNA for ddPCR should have only a SSB at the bottom strand within the F1/R1 amplicon, and the ddPCR should generate “+FAM+HEX droplets” because the top strand is intact (FIG. 16A). However, even when catalytically dead Cas9 (dCas9)¹² was used instead of eCas9 nickase in the Step 1, the fractions of −FAM+HEX droplets was 0.294±0.015 (FIG. 17C). Similarly, in the previous study of DSB-ddPCR, about 7% droplets also showed failed amplification of the F1/R1 amplicon and successful amplification of the F2/R2 amplicon even though the input DNA was uncleaved control DNA¹¹. It was speculated that the PCR amplification may fail in a fraction of droplets even though intact template DNA presents in the droplets, resulting in the “background level” of −FAM+HEX droplets.

To obtain the absolute value of the fraction of DNA cleaved by eCas9 nickase RNP in the Step 1, a standard curve was generated that can convert ddPCR readout (i.e., the fraction of “−FAM+HEX droplets”) into the absolute value of fraction of DNA (FIG. 16C). dCas9 RNP (using gMUC4-OneMM) was applied to fixed and permeabilized cells, and then genomic DNA were harvest (FIG. 18, Step 1). Next, the harvested genomic DNA was split into two tubes (FIG. 18, Step 1). One was treated with restriction enzyme (Msel) to generate “ds-cut genomic DNA”, and the other one was treated with Cas9 nickase RNP which cleaves the bottom strand to generate “ss-nicked genomic DNA” (FIG. 18, Step 2). Finally, “ds-cut genomic DNA” and “ss-nicked genomic DNA” were mixed at different ratios for ddPCR (FIG. 18). The relationship between ddPCR readout and the fraction of “ds-cut genomic DNA” added is linear (FIG. 16C, Pearson's r²>0.99). According to the standard curve, the fraction of DNA cleaved by eCas9 nickase RNP in the Step 1 of the SSB-ddPCR was insignificant (<0.05) for gMUC4-TwoMM and ˜0.4 for gMUC4-OneMM (FIG. 16C), consistent with the in vitro cleavage data using eCas9 RNP (FIG. 15D).

### REFERENCES

1. Zhang, K. et al. J Am Chem Soc 140, 11293-11301 (2018).
2. Janiszewska, M. et al. Nature genetics 47, 1212-1219 (2015).
3. Askary, A. et al. Nature biotechnology 38, 66-75 (2020).
4. Levsky, J. M. & Singer, R. H. Journal of cell science 116, 2833-2838 (2003).
5. Chen, B. et al. Cell 155, 1479-1491 (2013).
6. Lensing, S. V. et al. Nature methods 13, 855-857 (2016).
7. Tsai, S. Q. et al. Nature biotechnology 33, 187-197 (2015).
8. Wong, N., John, S., Nussenzweig, A. & Canela, A. in Homologous Recombination 9-31 (Springer US, 2021).
9. Ran, F. A. et al. Nature 520, 186-191 (2015).
10. Wu, W. et al. Nature (2021).
11. Rose, J. C. et al. Nature methods 14, 891-896 (2017).
12. Jinek, M. et al. Science 337, 816 (2012).

Lengthy table referenced here
US20250277256A1-20250904-T00001
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20250277256A1). An electronic copy of the table will also be available from the USPTO upon request and payment of the fee set forth in 37 CFR 1.19(b)(3).

1. A method of detecting a specific nucleic acid sequence in a genome, comprising:
inducing a nick in genomic nucleic acid sequences by a gene editing complex;
denaturing the genomic nucleic acid sequences by contacting the genomic nucleic acid sequences with a helicase enzyme at the nicked genomic nucleic acid sequences;
contacting the denatured genome with a detectably labeled probe, wherein the detectably labeled probe is complementary to the specific nucleic acid sequence of interest; and,
detecting the specific nucleic acid sequence of interest.

2. The method of claim 1, wherein the specific nucleic acid sequence of interest comprises one or more nucleic acid sequences in coding and non-coding nucleic acid sequences of the genome.

3. The method of claim 2, wherein the genomic nucleic acid sequences comprise genomic DNA.

4. The method of claim 1, wherein the nicking of genomic DNA sequences by the gene editing complex produces a 3′ single-stranded nucleic acid overhang.

5. The method of claim 4, wherein the helicase binds to the genomic DNA at the site of the nick and unwinds downstream double stranded genomic DNA.

6. The method of claim 1, wherein the gene editing complex comprises a Clustered Regularly Interspaced Short Palindromic Repeat (CRISPR)-associated endonuclease and at least one guide nucleic acid sequence.

7. The method of claim 1, wherein the gene editing complex comprises at least two guide nucleic acid sequences.

8. The method of claim 1, wherein the one or more guide nucleic acid sequences are RNA.

9. The method of claim 8, wherein the guide RNA (gRNA) sequences comprise at least about 90% sequence identity to one or more target nucleic acid sequences in coding and non-coding nucleic acid sequences of the genome, or complementary sequences thereof.

10. The method of claim 8, wherein the guide RNA (gRNA) sequences are complementary to one or more target nucleic acid sequences in coding and non-coding nucleic acid sequences of the genome, or complementary sequences thereof.

11. The method of claim 8, further comprising one or more guide RNAs having one or more nucleotide mismatches compared to the target nucleic acid sequence, or complementary sequences thereof.

12. The method of claim 11, wherein one or more single-nucleotide mismatches in one or more guide RNAs inhibit nicking of target genomic DNA.

13. The method of claim 1, wherein the guide RNA comprises crRNA and tracrRNA.

14. The method of claim 3, wherein the gene-editing complex comprises CRISPR-associated endonuclease is a Type I, Type II, or Type III Cas endonuclease.

15. The method of claim 6, wherein the CRISPR-associated endonuclease is a Cas9 endonuclease, a Cas12 endonuclease, a CasX endonuclease, a CasΦ endonuclease or variants thereof.

16. The method of claim 15, wherein the CRISPR-associated endonuclease is a Cas9 nuclease or variants thereof.

17. The method of claim 16, wherein the Cas9 nuclease is a Staphylococcus aureus Cas9 nuclease.

18-22. (canceled)

23. A method of detecting mutations in a genome of a cell or tissue, comprising:
inducing a nick in genomic DNA by a gene editing complex;
denaturing the genomic DNA by contacting the genome with a helicase enzyme at the nicked genomic DNA;
contacting the denatured genomic DNA with a detectably labeled probe, wherein the detectably labeled probe is complementary to the specific nucleic acid sequence of interest; and,
detecting the mutations in the genome.

24. The method of claim 23, wherein the specific nucleic acid sequence of interest comprises one or more nucleic acid sequences in coding and non-coding nucleic acid sequences of the genome.

25-45. (canceled)

46. A method of detecting single nucleotide variation (SNV) mutations in a genome of a cell or tissue, comprising:
inducing a nick in genomic DNA by a gene editing complex;
denaturing the genomic DNA by contacting the genome with a helicase enzyme at the nicked genomic DNA;
contacting the denatured genomic DNA with a detectably labeled probe, wherein the detectably labeled probe is complementary to the specific nucleic acid sequence of interest; and,
detecting the mutations in the genome.

47-61. (canceled)
